SEQUENCE LISTING (3 sequences)

SEQ ID NO: 1 - DNA, 21 bp, Artificial Sequence
  Oligonucleotide
ggcctcacagcgactctaaga

SEQ ID NO: 2 - DNA, 18 bp, Artificial Sequence
  Oligonucleotide
ctcggactcaccacaagc

SEQ ID NO: 3 - DNA, 19 bp, Artificial Sequence
  Oligonucleotide
gacggaggcaggccaagtg

(19) United States
(12) Patent Application Publication
(10) Pub. No.: US 2010/0184851 A1
(43) Pub. Date: Jul. 22, 2010

(54) INHIBITION OF CELL PROLIFERATION

(75) Inventors: Said M. Sebti, Tampa, FL (US); Srikumar Chellappan, Tampa, FL (US); Nicholas James Lawrence, Tampa, FL (US)

Correspondence Address: FISH & RICHARDSON P.C., PO BOX 1022, MINNEAPOLIS, MN, 55440-1022, US

(73) Assignee: University of South Florida, Tampa, FL (US)

(21) Appl. No.: 12/551,262

(22) Filed: Aug. 31, 2009

Related U.S. Application Data
(60) Provisional application No. 61/093,287, filed on Aug. 29, 2008.

(51) Int. Cl. A61K 31/155; C07C 335/32; C07D 317/60; A61K 31/36; A61P 35/00; C12N 5/00

(52) U.S. Cl. 514/466; 558/4; 514/508; 549/442; 435/375

(57) ABSTRACT

Compounds of formula (I) and (II) are provided as modulators of Rb:Raf-1 interactions which are potent, selective disruptors of Rb:Raf-1 binding. Therapeutic methods of using the compounds, for example for treating or ameliorating a cell proliferation disorder such as cancer, are provided.

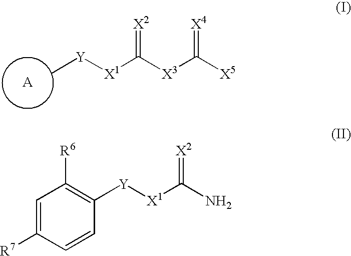

### CROSS-REFERENCE TO RELATED APPLICATIONS

This application claims the benefit of U.S. Provisional Application 61/093,287 filed Aug. 29, 2008, which is incorporated herein by reference in its entirety.

### GOVERNMENT SUPPORT

This invention was made with government support under grant numbers CA063136 and CA118210 awarded by the National Institutes of Health. The Government has certain rights in the invention.

### TECHNICAL FIELD

This application relates to compounds, pharmaceutical compositions, and methods for modulating the Rb:Raf-1 interaction in vitro or in vivo, and more particularly to treatment of disorders modulated by the Rb:Raf-1 interaction, for example, proliferation disorders such as cancer.

### BACKGROUND

Cellular proliferative orders such as cancer are among the most common causes of death in developed countries. For diseases for which treatments exist, despite continuing advances, the existing treatments often have undesirable side effects and limited efficacy. Identifying new effective drugs for cell proliferation disorders, including cancer, is a continuing focus of medical research.

### SUMMARY

The inactivation of the retinoblastoma tumor suppressor protein Rb by cell cycle regulatory kinases is disrupted in almost all cancers. In normal cells, inactivation of Rb is necessary for the G1 to S phase progression of the cell cycle. Raf-1 signaling kinase is known to play a role in promoting cancer, and studies have shown that Rb:Raf-1 binding facilitates cell proliferation.

The present disclosure relates to modulators of Rb:Raf-1 interactions that are surprisingly effective in inhibiting the tumor growth and survival of a wide variety of cancer cells. The application relates to compounds, pharmaceutical compositions, and methods for modulating cell proliferation and/or Rb:Raf-1 interaction in a cell, either in vitro or in vivo. For example, disorders that can be treated with the disclosed compounds, compositions, and methods include diseases such as cancer as well as non-cancerous proliferation disorders.

In one aspect, there is provided compound according to formula (I):

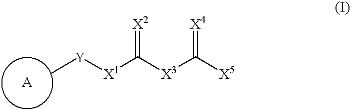

or a salt thereof, wherein:

Group A is substituted phenyl, optionally substituted 6-membered heteroaryl, or optionally substituted fused bicyclic 9-10 membered aryl or heteroaryl;

Y is optionally substituted methylene;

X¹ is —O—, —S—, or optionally substituted —NH—;

X³ is —O—, —S—, optionally substituted —NH— or optionally substituted methylene;

X² is S or optionally substituted NH;

X⁴ is S or optionally substituted NH;

or X² and X⁴ are both N and are linked together through an optionally substituted alkyl, alkenyl, heteroalkyl, or heteroalkenyl linking group, thereby forming an optionally substituted 5-7 membered heteroaryl or heterocyclyl ring; and

X⁵ is an optionally substituted —NH₂ or 3-7 membered heteroaryl or heterocyclyl ring;

wherein:

each optionally substitutable carbon is optionally substituted with —F, —Cl, —Br, —I, —CN, —NO₂, —R^{a}, —OR^{a}, —C(O)R^{a}, —OC(O)R^{a}, —C(O)OR^{a}, —SR^{a}, —C(S)R^{a}, —OC(S)R^{a}, —C(S)OR^{a}, —C(O)SR^{a}, —C(S)SR^{a}, —S(O)R^{a}, —SO₂R^{a}, —SO₃R^{a}, —OSO₂R^{a}, —OSO₃R^{a}, —PO₂R^{a}R^{b}, —OPO₂R^{a}R^{b}, —PO₃R^{a}R^{b}, —OPO₃R^{a}R^{b}, —N(R^{a}R^{b}), —C(O)N(R^{a}R^{b}), —C(O)NR^{a}NR^{b}SO₂R^{c}, —C(O)NR^{a}SO₂R^{c}, —C(O)NR^{a}CN, —SO₂N(R^{a}R^{b}), —NR^{a}SO₂R^{b}, —NR^{c}C(O)R^{a}, —NR^{c}C(O)OR^{a}, —NR^{c}C(O)N(R^{a}R^{b}), —C(NR^{c})—N(R^{a}R^{b}), —NR^{d}—C(NR^{c})—N(R^{a}R^{b}), —NR^{a}N(R^{a}R^{b}), —CR^{c}═CR^{a}R^{b}, —C≡CR^{a}, ═O, ═S, ═CR^{a}R^{b}, ═NR^{a}, ═NOR^{a}, or ═NNR^{a}, or two optionally substitutable carbons are linked with C₁₋₃ alkylenedioxy;

each optionally substitutable nitrogen is:

optionally substituted with —CN, —NO₂, —R^{a}, —OR^{a}, —C(O)R^{a}, —C(O)R^{a}-aryl, —OC(O)R^{a}, —C(O)OR^{a}, —SR^{a}, —S(O)R^{a}, —SO₂R^{a}, —SO₃R^{a}, —N(R^{a}R^{b}), —C(O)N(R^{a}R^{b}), —C(O)NR^{a}NR^{b}SO₂R^{c}, —C(O)NR^{a}SO₂R^{c}, —C(O)NR^{a}CN, —SO₂N(R^{a}R^{b}), —NR^{a}SO₂R^{b}, —NR^{c}C(O)R^{a}, —NR^{c}C(O)OR^{a}, —NR^{c}C(O)N(R^{a}R^{b}), or oxygen to form an N-oxide; and

is optionally protonated or quaternary substituted with a nitrogen substituent, thereby carrying a positive charge which is balanced by a pharmaceutically acceptable counterion; and

wherein each of R^{a}, R^{b}, R^{c} and R^{d} is independently —H, alkyl, haloalkyl, aralkyl, aryl, heteroaryl, heterocyclyl, or cycloaliphatic, or

in any occurrence of —N(R^{a}R^{b}), R^{a} and R^{b} taken together with the nitrogen to which they are attached optionally form an optionally substituted heterocyclic group

with the proviso that when X¹ is NH, X² is NH, X³ is NH, X⁴ is NH, X⁵ is NH₂, and Y is CH₂, then ring A is other than 2-trifluoromethylphenyl, 3-methoxyphenyl, 3-nitrophenyl, 3-trifluoromethylphenyl, 3-vinylphenyl, 4-t-butylphenyl, 4-chlorophenyl, 4-fluorophenyl, 4-methoxyphenyl, 4-methylphenyl, 4-nitrophenyl, 4-trifluoromethylphenyl, 4-vinylphenyl, 3,4-dichlorophenyl, 3,5-ditrifluoromethylphenyl, and 2-hydroxy-5-nitrophenyl.

In another aspect, there is provided a compound according to formula (II):

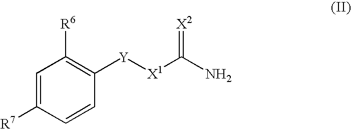

or a salt thereof, wherein:

Y is optionally substituted methylene;

X¹ is —O—, —S—, or optionally substituted —NH—; and

X² is S or optionally substituted NH;

R⁶ and R⁷ are independently —F, —Cl, —Br, —I, —NO₂, —CN, —CF₃, or C₁-C₆ alkoxy;

wherein

each optionally substitutable carbon is optionally substituted with —F, —Cl, —Br, —I, —CN, —NO₂, —R^{a}, —OR^{a}, —C(O)R^{a}, —OC(O)R^{a}, —C(O)R^{a}, —SR^{a}, —C(S)R^{a}, —OC(S)R^{a}, —C(S)OR^{a}, —C(O)SR^{a}, —C(S)SR^{a}, —S(O)R^{a}, —SO₂R^{a}, —SO₃R^{a}, —OSO₂R^{a}, —OSO₃R^{a}, —PO₂R^{a}R^{b}, —OPO₂R^{a}R^{b}, —PO₃R^{a}R^{b}, —OPO₃R^{a}R^{b}, —N(R^{a}R^{b}), —C(O)N(R^{a}R^{b}), —C(O)NR^{a}NR^{b}SO₂R^{c}, —C(O)NR^{a}SO₂R^{c}, —C(O)NR^{a}CN, —SO₂N(R^{a}R^{b}), —NR^{a}SO₂R^{b}, —NR^{c}C(O)R^{a}, —NR^{c}C(O)OR^{a}, —NR^{c}C(O)N(R^{a}R^{b}), —C(NR^{c})—N(R^{a}R^{b}), —NR^{d}—C(NR^{c})—N(R^{a}R^{b}), —NR^{a}N(R^{a}R^{b}), —CR^{c}═CR^{a}R^{b}, —C≡CR^{a}, ═O, ═S, ═CR^{a}R^{b}, ═NR^{a}, ═NOR^{a}, or ═NNR^{a}, or two optionally substitutable carbons are linked with C₁₋₃ alkylenedioxy;

each optionally substitutable nitrogen is:

optionally substituted with —CN, —NO₂, —R^{a}, —OR^{a}, —C(O)R^{a}, —C(O)R^{a}-aryl, —OC(O)R^{a}, —C(O)OR^{a}, —SR^{a}, —S(O)R^{a}, —SO₂R^{a}, —SO₃R^{a}, —N(R^{a}R^{b}), —C(O)N(R^{a}R^{b}), —C(O)NR^{a}NR^{b}SO₂R^{c}, —C(O)NR^{a}SO₂R^{c}, —C(O)NR^{a}CN, —SO₂N(R^{a}R^{b}), —NR^{a}SO₂R^{b}, —NR^{c}C(O)R^{a}, —NR^{c}C(O)OR^{a}, —NR^{c}C(O)N(R^{a}R^{b}), or oxygen to form an N-oxide; and

optionally is protonated or quaternary substituted with a nitrogen substituent, thereby carrying a positive charge which is balanced by a pharmaceutically acceptable counterion; and

wherein each of R^{a}, R^{b}, R^{c} and R^{d} is independently —H, alkyl, haloalkyl, aralkyl, aryl, heteroaryl, heterocyclyl, or cycloaliphatic, or

in any occurrence of —N(R^{a}R^{b}), R^{a} and R^{b} taken together with the nitrogen to which they are attached optionally form an optionally substituted heterocyclic group.

In some embodiments of the compounds of formula II, R⁶ and R⁷ are not both —Cl and R⁶ and R⁷ are not both —CF₃.

In some embodiments of the compounds of formula II, when Y is —CH₂—, X¹ is S and X² is NH, then R⁶ and R⁷ are not both —F, R⁶ and R⁷ are not both —Br, R⁶ and R⁷ are not both —I, R¹ and R² are not both —NO₂; and R⁶ and R⁷ are not both —CH₃;

In some embodiments of the compounds of formula II, R⁶ and R⁷ are not both —F, R⁶ and R⁷ are not both —Br, R⁶ and R⁷ are not both —I, R⁶ and R⁷ are not both —NO₂, and R⁶ and R⁷ are not both —CH₃.

In some embodiments of the compounds of formula II, Y is C(O), C(S), or methylene optionally substituted with hydroxyl, C₁₋₆ alkyl, C₁₋₆ alkoxy, C₁₋₆ haloalkyl, C₁₋₆ haloalkoxy, C₁₋₆ alkyl substituted with aryl, aryl, heteroaryl, heterocyclyl, or cycloaliphatic. In some embodiments, Y is methylene optionally substituted with hydroxyl, C₁₋₆ alkyl, C₁₋₆ alkoxy, or C₁₋₆ alkyl substituted with aryl. In some embodiments, Y is methylene optionally substituted with C₁₋₃ alkyl, for example methyl. In some embodiments, Y is methylene.

Also provided are methods of using the disclosed compounds. The disclosed compounds are useful in inhibiting the Rb-Raf-1 binding. The disclosed compounds are biologically active and therapeutically useful.

The compounds, pharmaceutical compositions, and methods of treatment described in this application are believed to be effective for inhibiting cellular proliferation, particularly of cells which proliferate due to a mutation or other defect in the Rb:Raf-1 regulatory pathway. The disclosed compounds, pharmaceutical compositions, and methods of treatment are therefore believed to be effective for treating cancer and other proliferative disorders which can be inhibited by disrupting Rb:Raf-1 binding interactions in the proliferating cells.

A method of inhibiting proliferation of a cell is provided. The method includes contacting the cell with an effective amount of one of the disclosed compounds, or a pharmaceutically acceptable salt thereof.

A method of modulating Rb:Raf-1 binding in a proliferating cell is provided. The method includes contacting the cell with an effective amount of one of the disclosed compounds, or a pharmaceutically acceptable salt thereof.

A method of treating or ameliorating a cell proliferation disorder is provided. The method includes contacting proliferating cells with an effective amount of one of the disclosed compounds, or a pharmaceutically acceptable salt thereof.

A method of treating or ameliorating a cell proliferation disorder is provided. The method includes administering to a subject in need of such treatment an effective amount of a compound according to any one of the disclosed compounds, or a pharmaceutically acceptable salt thereof.

A method is provided for inhibiting angiogenic tubule formation in a subject in need thereof. The method includes administering to the subject an effective amount of one of the disclosed compounds, or a pharmaceutically acceptable salt thereof.

A method is provided for assessing a subject for treatment with an inhibitor of Rb:Raf-1 binding interactions. The method includes determining, in the subject or in a sample from the subject, a level of Rb, Raf-1, or Rb bound to Raf-1, wherein treatment with an inhibitor of Rb:Raf-1 binding interactions is indicated when the level of Rb, Raf-1, or Rb bound to Raf-1 is elevated compared to normal. The inhibitor of Rb:Raf-1 binding interactions is one of the disclosed compounds, or a pharmaceutically acceptable salt thereof.

A method is provided for identifying a subject for therapy. The method includes obtaining a sample from the subject; determining a level of Rb, Raf-1, or Rb bound to Raf-1 in the sample; and identifying the subject for therapy with an inhibitor of Rb:Raf-1 binding interactions when the level of Rb, Raf-1, or Rb bound to Raf-1 is elevated compared to normal. The inhibitor of Rb:Raf-1 binding interactions is one of the disclosed compounds, or a pharmaceutically acceptable salt thereof

Also provided are pharmaceutical compositions including the disclosed compounds, or pharmaceutically acceptable salts thereof and a pharmaceutically acceptable carrier.

The disclosed compounds may be provided for use in of the therapeutic methods described herein.

Also provided is the use of the disclosed compounds, or pharmaceutically acceptable salts thereof, for the manufacture of a medicament for carrying out the therapeutic methods described herein.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1A: Identification of Rb:Raf-1 inhibitors. An immunoprecipitation-western blot analysis showing the disruption of the Rb:Raf-1 interaction by compounds 10b and 10c.

FIG. 1B: BrdU incorporation assay showing that compound 10b arrests wild-type A549 cells, but Rb is required for activity of compound 10b; 5, 10 and 20 μM of 10b does not inhibit the proliferation of A549 cells over-expressing shRNA constructs to Rb (sh6 and sh8), but 10b arrests wild-type A549 cells.

FIG. 1C: BrdU incorporation assay showing that compound 10c arrests wild-type A549 cells, but Rb is required for activity of compound 10c; 5, 10 and 20 μM of 10c does not inhibit the proliferation of A549 cells over-expressing shRNA constructs to Rb (sh6 and sh8), but 10c arrests wild-type A549 cells.

FIG. 1D: A BrdU incorporation assay at compound concentrations of 5, 10, 20, 30 and 50 μM shows dose-dependent inhibition of wild-type A549 cells by compounds 3w, 10a, 10b and 10c.

FIG. 1E: Compounds 10b and 10c inhibit angiogenic tubule formation in matrigel in a dose-dependent fashion as shown at concentrations of 20, 50 and 100 μM. For comparison, lack of inhibition of angiogenic tubule formation in matrigel is shown for control-no drug, and comparable inhibition is shown by compound 3a at 100 μM.

FIG. 1F: Compounds 10b and 10c at 150 mg/kg inhibit human tumor growth in nude mice. A549 cells xenotransplanted bilaterally into the flanks of athymic nude mice were allowed to grow for 14 days until tumor volume reached 200 mm³; daily administration of compounds 10b and 10c substantially inhibited tumor growth whereas control tumors grew to almost 1200 mm³.

FIG. 1G: Compound 10c inhibited the proliferation of a wide range of cancer cells at 20 μM. In a BrdU incorporation assay, compound 10c was contacted with a range of cancer cells including PANC-1 (human pancreatic carcinoma, epithelial-like), CAPAN-2 (human pancreatic ductal adenocarcinoma), Mel-5 (human malignant melanoma), MCF-7 (human breast adenocarcinoma), LNCAP (androgen-sensitive human prostate adenocarcinoma), A549 (human epithelial lung carcinoma), and PC-3 (human prostate adenocarcinoma), and compared to Rb-deficient cancer cells (A549 cells stably transfected with two different shRNA constructs (sh6 and sh8) to knock down Rb expression, and the Rb-deficient prostate cancer cell line DU145). This result confirms that compound 10c arrests the proliferation of a wide variety of cancer cells in a Rb dependent manner.

FIG. 2: Results of a MTT assay in which U937 myeloid cells were incubated in the absence of compound (control), or with compounds 3a, 10b, or 10c at 10 μM, 20 μM, or 50 μM for 24 hours showing dose-dependent reduction in viability of the cancer cells in the presence of the compound.

FIG. 3: Results of a MTT assay in which Ramos cells (Burkitt's Lymphoma) were incubated in the absence of compound (control), or with compounds 3a, 10b, or 10c at 10 μM, 20 μM, or 50 μM for 24 hours showing dose-dependent reduction in viability of the cancer cells in the presence of the compound.

FIG. 4: Results of a BrdU incorporation assay where cells lacking Raf-1 due to presence of a Raf-inhibitory shRNA or control cells (containing a control shRNA) were incubated in the presence or absence of compounds 3a, 10b and 10c (20 μM). The compounds inhibit the proliferation of cells having Raf-1 but not the cells lacking Raf-1.

FIG. 5A. A schematic of the promoters showing the E2F binding site on the genes for MMP2, MMP9 and MMP14.

FIG. 5B. Results of a QRT-PCR experiment measuring the expression of MMP2, MMP9 and MMP14 in A549 cells transfected with shRNA to inhibit expression of ECF1 or control cells. When expression of ECF1 is depleted, the expression of MMP9 and MMP14 is reduced.

FIGS. 6A-D. Results of a chromatin immunoprecipitation assay showing the binding of ECF1 and the association of Rb with promoters of matrix metalloproteinases MMP2 (FIG. 6A), MMP9 (FIG. 6B), MMP14 (FIG. 6C), and MMP15 (FIG. 6D)

FIGS. 7A-D. Results of a QRT-PCT experiment performed to measure the effect of compounds 3a, 10b and 10c on the expression of FIGS. 7A (MMP2), 7B (MMP9), 7C (MMP14) and 7D (MMP15) in MDAMB231 cells (breast cancer) showing expression of MMP9, MMP14 and MMP15 inhibited by each of the compounds.

FIG. 8A. A schematic diagram showing E2F binding sites on the promoters for VEGF receptors, FLT1 and KDR.

FIGS. 8B-D show the results of chromatin immunoprecipitation assay performed using primary endothelial cells: human aortic endothelial cells HAEC (FIG. 8B), human umbilical cord vein endothelial cell (HUVEC) (FIG. 8C) and human microvascular endothelial cells from the lung (HMEC-L) (FIG. 8D). Treatment of the primary endothelial cells (human aortic endothelial cells, human umbilical cord vein endothelial cells or human microvascular endothelial cells from the lung) with VEGF induced the binding of E2F1 to the FLT1 and KDR promoters.

FIG. 9 shows data demonstrating that transient transfection of E2F1 induces FLT1 and KDR promoters and that Rb can repress these promoters. The transfection assays were performed in both A549 and HUVEC cells.

FIG. 10 shows the results of a QRT-PCR experiments performed to measure the effect of compounds 3a, 10b and 10c (50 μM) on the expression of FLT1 and KDR in human aortic endothelial cells. Each of the compounds inhibits expression of both FLT and KDR.

### DETAILED DESCRIPTION

This application relates to compounds, pharmaceutical compositions, and methods for modulating cell proliferation and/or Rb:Raf-1 interaction in a cell, either in vitro or in vivo. For example, disorders that can be treated with the disclosed compounds, compositions, and methods include diseases such as cancer as well as non-cancerous proliferation disorders. Without wishing to be bound by any theory, it is believed that the pharmaceutical activity of the disclosed compounds arises, at least in part, to modulation of Rb:Raf-1 binding interactions by the disclosed compound, and more particularly to disruption of Rb:Raf-1 binding.

In various embodiments, the disclosed compounds are modulators of Rb:Raf-1 binding interactions. A modulator can change the action or activity of the molecule, enzyme, or system which it targets. For example, the disclosed modulators can modulate Rb:Raf 1 binding interactions to inhibit, disrupt, prevent, block or antagonize Rb, Raf-1, or Rb:Raf-1 binding interactions, or otherwise prevent association or interaction between Rb and Raf-1. Thus, the disclosed compounds can be inhibitors, disruptors, blockers, or antagonists of Rb or Raf-1 activity, or of Rb:Raf-1 binding interactions.

Thus, the compounds, pharmaceutical compositions, and methods of use described in this application are believed to be effective for inhibiting cellular proliferation, particularly of cells which proliferate due to a mutation or other defect in the Rb:Raf-1 regulatory pathway. In particular, the disclosed compounds, pharmaceutical compositions, and methods of use are believed to be effective for treating cancer and other proliferative disorders which can be inhibited by disrupting Rb:Raf-1 binding interactions in the proliferating cells.

The inactivation of the retinoblastoma tumor suppressor protein Rb by cell cycle regulatory kinases is disrupted in almost all cancers. In normal cells, inactivation of Rb is necessary for the G1 to S phase progression of the cell cycle. Rb controls entry into the S phase by repressing the transcriptional activity of the E2F family of transcription factors, especially E2Fs 1, 2, and 3. Rb is inactivated through multiple phosphorylation events mediated by kinases associated with D and E type cyclins in the G1 phase of the cell cycle. It was found that the signaling kinase Raf-1 initiates the phosphorylation events; Raf-1 signaling kinase is known to play a role in promoting cancer, and studies have shown that Rb:Raf-1 binding facilitates cell proliferation. It has also been found that the Rb:Raf-1 interaction is elevated in human tumors compared to adjacent normal tissue in 80% of samples examined. Because Raf-1 is persistently activated in many tumors, a few attempts have been made to selectively inhibit tumors by modulating Rb and/or Raf-1 activity with Raf-1 antisense oligonucleotides, the multikinase inhibitor Sorafenib, and a peptide fragment of Raf-1 coupled to a carrier peptide. However, there is still a need for effective modulators of the Rb:Raf-1 interaction.

Without being bound by any theory, it has been found that modulators of Rb:Raf-1 interactions that are surprisingly effective in inhibiting the tumor growth and survival of a wide variety of cancer cells. For example, modulators of Rb:Raf 1 interactions are potent, selective disruptors of Rb:Raf-1 binding. Also, modulators of Rb:Raf 1 interactions are surprisingly effective in inhibiting the tumor growth and survival of a wide variety of cancer cells, including osteosarcoma, epithelial lung carcinoma, non-small cell lung carcinoma, three different pancreatic cancer cell lines, glioblastoma cell lines, metastatic breast cancer, melanoma, and prostate cancer. Moreover, modulators of Rb:Raf 1 interactions effectively disrupt angiogenesis, significantly inhibited anchorage independent tumor and significantly inhibited the growth of human epithelial lung carcinoma in nude mice. Accordingly, compounds, pharmaceutical compositions comprising the compounds, methods of inhibiting cell proliferation, methods of treating subjects with cancer, and methods of preparing modulators of Rb:Raf 1 interactions are provided herein.

### I. DEFINITIONS

### A. General

As used in the specification and the appended claims, the singular forms “a,” “an” and “the” include plural referents unless the context clearly dictates otherwise.

The term “contacting” means bringing at least two moieties together, whether in an in vitro system or an in vivo system.

The term “cell proliferation disorder” means a disorder wherein unwanted cell proliferation of one or more subsets of cells in a multicellular organism occurs. In some such disorders, cells are made by the organism at an atypically accelerated rate. The term includes cancer and non-cancerous cell proliferation disorders. In some embodiments, the cell proliferation disorder is angiogenesis or the cell proliferation disorder is mediated by angiogenesis.

The expression “effective amount”, when used to describe an amount of compound or radiation applied in a method, refers to the amount of a compound that achieves the desired pharmacological effect or other effect, for example an amount that inhibits the abnormal to growth or proliferation, or induces apoptosis of cancer cells, resulting in a useful effect.

The terms “treating” and “treatment” mean causing a therapeutically beneficial effect, such as ameliorating existing symptoms, preventing additional symptoms, ameliorating or preventing the underlying metabolic causes of symptoms, postponing or preventing the further development of a disorder and/or reducing the severity of symptoms that will or are expected to develop.

As used herein, “individual” (as in the subject of the treatment) means both mammals and non-mammals. Mammals include, for example, humans; non-human primates, e.g. apes and monkeys; cattle; horses; sheep; rats; mice; pigs; and goats. Non-mammals include, for example, fish and birds.

As used herein, the term “pharmaceutically acceptable” means that the materials (e.g., compositions, carriers, diluents, reagents, salts, and the like) are capable of administration to or upon a mammal with a minimum of undesirable physiological effects such as nausea, dizziness or gastric upset.

### B. Chemical

In the following paragraphs some of the definitions include examples. The examples are intended to be illustrative, and not limiting. When a term defined below is used in the specification, it is to be understood that the term includes the embodiments encompassed by the term, including the exemplary embodiments described herein.

An aliphatic group is a straight chained, branched non-aromatic hydrocarbon which is completely saturated or which contains one or more units of unsaturation. A cycloaliphatic group is an aliphatic group that forms a ring. Alkyl and cycloalkyl groups are saturated aliphatic and saturated cycloaliphatic groups, respectively. Typically, a straight chained or branched aliphatic group has from 1 to about 10 carbon atoms, typically from 1 to about 6, and preferably from 1 to about 4, and a cyclic aliphatic group has from 3 to about 10 carbon atoms, typically from 3 to about 8, and preferably from 3 to about 6. An aliphatic group is preferably a straight chained or branched alkyl group, e.g., methyl, ethyl, n-propyl, iso-propyl, n-butyl, sec-butyl, tert-butyl, pentyl, hexyl, pentyl or octyl, or a cycloalkyl group with 3 to about 8 carbon atoms. C₁₋₆ straight chained or branched alkyl or alkoxy groups or a C₃₋₈ cyclic alkyl or alkoxy group (preferably C₁₋₆ straight chained or branched alkyl or alkoxy group) are also referred to as a “lower alkyl” or “lower alkoxy” groups; such groups substituted with —F, —Cl, —Br, or —I are “lower haloalkyl” or “lower haloalkoxy” groups; a “lower hydroxyalkyl” is a lower alkyl substituted with —OH; and the like.

The term “alkyl” or “(C_{x-y})alkyl” (wherein x and y are integers) by itself or as part of another substituent means, unless otherwise stated, an alkyl group containing between x and y carbon atoms. An alkyl group formally corresponds to an alkane or cycloalkane with one C—H bond replaced by the point of attachment of the alkyl group to the remainder of the compound. An alkyl group may be straight-chained or branched. Alkyl groups having 3 or more carbon atoms may be cyclic. Cyclic alkyl groups having 7 or more carbon atoms may contain more than one ring and be polycyclic. Examples of straight-chained alkyl groups include methyl, ethyl, n-propyl, n-butyl, and n-octyl. Examples of branched alkyl groups include i-propyl, t-butyl, and 2,2-dimethylethyl. Examples of cyclic alkyl groups include cyclopentyl, cyclohexyl, cyclohexylmethyl, and 4-methylcyclohexyl. Examples of polycyclic alkyl groups include bicyclo[2.2.1]heptanyl, norbornyl, and adamantyl. Examples of alkyl and (C_{x-y})alkyl groups are (C₁₋₆)alkyl such as (C₁₋₃)alkyl, for example methyl and ethyl.

The term “alkylene” or “(C_{x-y})alkylene” (wherein x and y are integers) refers to an alkylene group containing between x and y carbon atoms. An alkylene group formally corresponds to an alkane with two C—H bond replaced by points of attachment of the alkylene group to the remainder of the compound. Included are divalent straight hydrocarbon group consisting of methylene groups, such as, —CH₂—, —CH₂CH₂—, —CH₂CH₂CH₂—. In some embodiments, alkylene or (C_{x-y})alkylene may be (C₁₋₆)alkylene such as (C₁₋₃)alkylene.

The term “alkenyl” or “(C_{x-y}) alkenyl” (wherein x and y are integers) denotes a radical containing x to y carbons, wherein at least one carbon-carbon double bond is present (therefore x must be at least 2). Some embodiments are 2 to 4 carbons, some embodiments are 2 to 3 carbons, and some embodiments have 2 carbons. Both E and Z isomers are embraced by the term “alkenyl.” Furthermore, the term “alkenyl” includes di- and tri-alkenyls. Accordingly, if more than one double bond is present then the bonds may be all E or Z or a mixtures of E and Z. Examples of an alkenyl include vinyl, allyl, 2-butenyl, 3-butenyl, 2-pentenyl, 3-pentenyl, 4-pentenyl, 2-hexenyl, 3-hexenyl, 4-hexenyl, 5-hexanyl, 2,4-hexadienyl and the like.

The term “alkynyl” or “(C_{x-y}) alkynyl” (wherein x and y are integers) denotes a radical containing 2 to 6 carbons and at least one carbon-carbon triple bond, some embodiments are 2 to 4 carbons, some embodiments are 2 to 3 carbons, and some embodiments have 2 carbons. Examples of an alkynyl include ethynyl, ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, 5-hexynyl and the like. The term “alkynyl” includes di- and tri-ynes.

The term “alkoxy” or “(C_{x-y}) alkoxy” (wherein x and y are integers) employed alone or in combination with other terms means, unless otherwise stated, an alkyl group having the designated number of carbon atoms, as defined above, connected to the rest of the molecule via an oxygen atom, such as, for example, methoxy, ethoxy, 1-propoxy, 2-propoxy (isopropoxy) and the higher homologs and isomers. Embodiments include (C₁₋₃)alkoxy, such as ethoxy and methoxy.

The term “haloalkyl” or “(C_{x-y})haloalkyl” (wherein x and y are integers) by itself or as part of another substituent means, unless otherwise stated, an alkyl group or (C_{x-y})alkyl group in which a halogen is substituted for one or more of the hydrogen atoms. Examples include trifluoromethyl, 2,2,2-trifluoroethyl and trichloromethyl.

An “alkylene” group is a linking alkyl chain represented by —(CH₂)ₙ-, wherein n, the number of “backbone” atoms in the chain, is an integer from 1-10, typically 1-6, and preferably 1-4. An “alkenylene” group is a linking alkyl chain having one or more double bonds, wherein the number of backbone atoms is an integer from 1-10, typically 1-6, and preferably 1-4. An “alkynylene” group is a linking alkyl chain having one or more triple bonds and optionally one or more double bonds, wherein the number of “backbone” atoms is an integer from 1-10, typically 1-6, and preferably 1-4.

“Heteroalkylene,” “heteroalkenylene,” and “heteroalkynylene” groups are alkylene, alkenylene, and alkynylene groups, respectively, wherein one or more carbons are replaced with heteroatoms such as N, O, or S.

A “heterocyclic group” or “heterocyclyl” is a non-aromatic cycloaliphatic group which has from 3 to about 10 ring atoms, typically from 3 to about 8, and preferably from 3 to about 6, wherein one or more of the ring atoms is a heteroatom such as N, O, or S in the to ring. Examples of heterocyclic groups include oxazolinyl, thiazolinyl, oxazolidinyl, thiazolidinyl, tetrahydrofuranyl, tetrahydrothiophenyl, morpholino, thiomorpholino, pyrrolidinyl, piperazinyl, piperidinyl, thiazolidinyl, and the like.

Examples of non-aromatic heterocycles also include monocyclic groups such as: aziridine, oxirane, thiirane, azetidine, oxetane, thietane, pyrrolidine, pyrroline, imidazoline, pyrazolidine, dioxolane, sulfolane, 2,3-dihydrofuran, 2,5-dihydrofuran, tetrahydrofuran, thiophane, piperidine, 1,2,3,6-tetrahydropyridine, 1,4-dihydropyridine, piperazine, morpholine, thiomorpholine, pyran, 2,3-dihydropyran, tetrahydropyran, 1,4-dioxane, 1,3-dioxane, homopiperazine, homopiperidine, 1,3-dioxepane, 4,7-dihydro-1,3-dioxepin and hexamethyleneoxide.

The term “aromatic” refers to a carbocycle or heterocycle having one or more polyunsaturated rings having aromatic character (i.e. having (4n+2) delocalized π (pi) electrons where n is an integer).

The term “aryl”, employed alone or in combination with other terms, means, unless otherwise stated, a carbocyclic aromatic system containing one or more rings (typically one, two or three rings), wherein such rings may be attached together in a pendent manner, such as a biphenyl, or may be fused, such as naphthalene. Examples include phenyl; anthracyl; and naphthyl. Preferred are phenyl and naphthyl, most preferred is phenyl. In some embodiments, the term refers to C₆₋₁₄ carbocyclic aromatic groups such as phenyl, biphenyl, and the like. Aryl groups also include fused polycyclic aromatic ring systems in which a carbocyclic aromatic ring is fused to other aryl, cycloalkyl, or cycloaliphatic rings, such as naphthyl, pyrenyl, anthracyl, 9,10-dihydroanthracyl, fluorenyl, and the like.

The term “aralkyl” or “aryl-(C_{x-y})alkyl” means a functional group wherein carbon alkylene chain of x to y carbon atoms is attached to an aryl group, e.g., —CH₂CH₂-phenyl. Examples include is aryl(CH₂)— (e.g. benzyl) and aryl(CH(CH₃))-. The term “substituted aralkyl” or “substituted aryl-(C₁₋₃)alkyl” means an aryl-(C₁₋₃)alkyl functional group in which the aryl group is substituted. Preferred is substituted aryl(CH₂)—. Similarly, the term “heteroaryl(C₁₋₃)alkyl” means a functional group wherein a one to three carbon alkylene chain is attached to a heteroaryl group, e.g., —CH₂CH₂-pyridyl. Preferred is heteroaryl(CH₂)—. The term “substituted heteroaryl-(C₁₋₃)alkyl” means a heteroaryl-(C₁-C₃)alkyl functional group in which the heteroaryl group is substituted. Preferred is substituted heteroaryl(CH₂)—.

The term “heteroaryl” refers to 5-14 membered aryl groups having 1 or more O, S, or N heteroatoms. Examples of heteroaryl groups include pyridyl, pyrimidyl, pyrazinyl, triazinyl, pyranyl, pyrrolyl, imidazolyl, pyrazolyl, 1,2,3-trizaolyl, 1,2,4-triazolyl, tetrazolyl, thienyl, thiazoyl, isothiazolyl, furanyl, oxazolyl, isooxazolyl, and the like. Heteroaryl groups also include fused polycyclic aromatic ring systems in which a carbocyclic aromatic ring or heteroaryl ring is fused to one or more other heteroaryl rings. Examples include quinolinyl, isoquinolinyl, quinazolinyl, napthyridyl, pyridopyrimidyl, benzothienyl, benzothiazolyl, benzoisothiazolyl, thienopyridyl, thiazolopyridyl, isothiazolopyridyl, benzofuranyl, benzooxazolyl, benzoisooxazolyl, furanopyridyl, oxazolopyridyl, isooxazolopyridyl, indolyl, isoindolyl, benzimidazolyl, benzopyrazolyl, pyrrolopyridyl, isopyrrolopyridyl, imidazopyridyl, pyrazolopyridyl, and the like. A ring recited as a substituent herein can be bonded via any substitutable atom in the ring.

Examples of heteroaryl groups include: pyridyl, pyrazinyl, pyrimidinyl, particularly 2- and 4-pyrimidinyl, pyridazinyl, thienyl, furyl, pyrrolyl, particularly 2-pyrrolyl, imidazolyl, thiazolyl, oxazolyl, pyrazolyl, particularly 3- and 5-pyrazolyl, isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl and 1,3,4-oxadiazolyl.

Examples of polycyclic heterocycles include: indolyl, particularly 3-, 4-, 5-, 6- and 7-indolyl, indolinyl, quinolyl, tetrahydroquinolyl, isoquinolyl, particularly 1- and 5-isoquinolyl, 1,2,3,4-tetrahydroisoquinolyl, cinnolinyl, quinoxalinyl, particularly 2- and 5-quinoxalinyl, quinazolinyl, phthalazinyl, 1,5-naphthyridinyl, 1,8-naphthyridinyl, 1,4-benzodioxanyl, coumarin, dihydrocoumarin, benzofuryl, particularly 3-, 4-, 5-, 6- and 7-benzofuryl, 2,3-dihydrobenzofuryl, 1,2-benzisoxazolyl, benzothienyl, particularly 3-, 4-, 5-, 6-, and 7-benzothienyl, benzoxazolyl, benzthiazolyl, particularly 2-benzothiazolyl and 5-benzothiazolyl, purinyl, benzimidazolyl, particularly 2-benzimidazolyl, benztriazolyl, thioxanthinyl, carbazolyl, carbolinyl, acridinyl, pyrrolizidinyl, and quinolizidinyl.

The aforementioned listing of heterocyclyl and heteroaryl moieties is intended to be representative and not limiting.

The term “substituted” means that an atom or group of atoms formally replaces hydrogen as a “substituent” attached to another group. For aryl and heteroaryl groups, the term “substituted”, unless otherwise indicated, refers to any level of substitution, namely mono-, di-, tri-, tetra-, or penta-substitution, where such substitution is permitted. The substituents are independently selected, and substitution may be at any chemically accessible position.

The “valency” of a chemical group refers to the number of bonds by which it is attached to other groups of the molecule.

Suitable optional substituents for a substitutable atom in the preceding groups, e.g., alkyl, cycloalkyl, aliphatic, cycloaliphatic, alkylene, alkenylene, alkynylene, heteroalkylene, heteroalkenylene, heteroalkynylene, heterocyclic, aryl, and heteroaryl groups, are those substituents that do not substantially interfere with the pharmaceutical activity of the disclosed compounds. A “substitutable atom” is an atom that has one or more valences or charges available to form one or more corresponding covalent or ionic bonds with a substituent. For example, a carbon atom with one valence available (e.g., —C(—H)═) can form a single bond to an alkyl group (e.g., —C(-alkyl)═), a carbon atom with two valences available (e.g., —C(H₂)—) can form one or two single bonds to one or two substituents (e.g., —C(alkyl)(H)—, —C(alkyl)(Br))-,) or a double bond to one substituent (e.g., —C(═O)—), and the like. Substitutions contemplated herein include only those substitutions that form stable compounds.

For example, suitable optional substituents for substitutable carbon atoms include —F, —Cl, —Br, —I, —CN, —NO₂, —OR^{a}, —C(O)R^{a}, —OC(O)R^{a}, —C(O)OR^{a}, —SR^{a}, —C(S)R^{a}, —OC(S)R^{a}, —C(S)OR^{a}, —C(O)SR^{a}, —C(S)SR^{a}, —S(O)R^{a}, —SO₂R^{a}, —SO₃R^{a}, —OSO₂R^{a}, —OSO₃R^{a}, —PO₂R^{a}R^{b}, —OPO₂R^{a}R^{b}, —PO₃R^{a}R^{b}, —OPO₃R^{a}R^{b}, —N(R^{a}R^{b}), —C(O)N(R^{a}R^{b}), —C(O)NR^{a}NR^{b}SO₂R^{c}, —C(O)NR^{a}SO₂R^{c}, —C(O)NR^{a}CN, —SO₂N(R^{a}R^{b}), —SO₂N(R^{a}R^{b}), —NR^{c}C(O)R^{a}, —NR^{c}C(O)OR^{a}, —NR^{c}C(O)N(R^{a}R^{b}), —C(NR^{c})—N(R^{a}R^{b}), —NR^{d}—C(NR^{c})—N(R^{a}R^{b}), —NR^{a}N(R^{a}R^{b}), —CR^{c}═CR^{a}R^{b}, —C≡CR^{a}, ═O, ═S, ═CR^{a}R^{b}, ═NR^{a}, ═NOR^{a}, ═NNR^{a}, optionally substituted alkyl, optionally substituted cycloalkyl, optionally substituted aliphatic, optionally substituted cycloaliphatic, optionally substituted heterocyclic, optionally substituted benzyl, optionally substituted aryl, and optionally substituted heteroaryl, wherein R^{a}-R^{d} are each independently —H or an optionally substituted aliphatic, optionally substituted cycloaliphatic, optionally substituted heterocyclic, optionally substituted benzyl, optionally substituted aryl, or optionally substituted heteroaryl, or, —N(R^{a}R^{b}), taken together, is an optionally substituted heterocyclic group.

Suitable substituents for nitrogen atoms having two covalent bonds to other atoms include, for example, optionally substituted alkyl, optionally substituted cycloalkyl, optionally substituted aliphatic, optionally substituted cycloaliphatic, optionally substituted heterocyclic, optionally substituted benzyl, optionally substituted aryl, optionally substituted heteroaryl, —CN, —NO₂, —OR^{a}, —C(O)R^{a}, —OC(O)R^{a}, —C(O)OR^{a}, —SR^{a}, —S(O)R^{a}, —SO₂R^{a}, —SO₃R^{a}, —N(R^{a}R^{b}), —C(O)N(R^{a}R^{b}), —C(O)NR^{a}NR^{b}SO₂R^{c}, —C(O)NR^{a}SO₂R^{c}, —C(O)NR^{a}CN, —SO₂N(R^{a}R^{b}), —SO₂N(R^{a}R^{b}), —NR^{c}C(O)R^{a}, —NR^{c}C(O)OR^{a}, —NR^{c}C(O)N(R^{a}R^{b}), and the like.

A nitrogen-containing group, for example, a heteroaryl or non-aromatic heterocycle, can be substituted with oxygen to form an N-oxide, e.g., as in a pyridyl N-oxide, piperidyl N-oxide, and the like. For example, in various embodiments, a ring nitrogen atom in a nitrogen-containing heterocyclic or heteroaryl group can be substituted to form an N-oxide.

Suitable substituents for nitrogen atoms having three covalent bonds to other atoms include —OH, alkyl, and alkoxy (preferably C₁₋₆ alkyl and alkoxy). Substituted ring nitrogen atoms that have three covalent bonds to other ring atoms are positively charged, which is balanced by counteranions corresponding to those found in pharmaceutically acceptable salts, such as chloride, bromide, fluoride, iodide, formate, acetate and the like. Examples of other suitable counteranions are provided in the section below directed to suitable pharmacologically acceptable salts.

### II. COMPOUNDS

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable subcombination.

In one aspect, there is provided a compound according to formula (I):

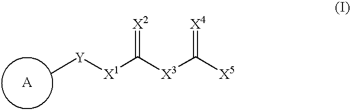

or a salt such as a pharmaceutically acceptable salt thereof, wherein:

Group A is substituted phenyl, optionally substituted 6-membered heteroaryl, or optionally substituted fused bicyclic 9-10 membered aryl or heteroaryl;

Y is optionally substituted methylene;

X¹ is —O—, —S—, or optionally substituted —NH—;

X³ is —O—, —S—, optionally substituted —NH— or optionally substituted methylene;

X² is S or optionally substituted NH;

X⁴ is S or optionally substituted NH;

or X² and X⁴ are both N and are linked together through a bond or an optionally substituted alkyl, alkenyl, heteroalkyl, or heteroalkenyl linking group, thereby forming an optionally substituted 5-7 membered heteroaryl or heterocyclyl ring; and

X⁵ is an optionally substituted —NH₂ or 3-7 membered heteroaryl or heterocyclyl ring;

wherein

each optionally substitutable carbon is optionally substituted with —F, —Cl, —Br, —I, —CN, —NO₂, —R^{a}, —OR^{a}, —C(O)R^{a}, —OC(O)R^{a}, —C(O)OR^{a}, —SR^{a}, —C(S)R^{a}, —OC(S)R^{a}, —C(S)OR^{a}, —C(O)SR^{a}, —C(S)SR^{a}, —S(O)R^{a}, —SO₂R^{a}, —SO₃R^{a}, —OSO₂R^{a}, —OSO₃R^{a}, —PO₂R^{a}R^{b}, —OPO₂R^{a}R^{b}, —PO₃R^{a}R^{b}, —OPO₃R^{a}R^{b}, —N(R^{a}R^{b}), —C(O)N(R^{a}R^{b}), —C(O)NR^{a}NR^{b}SO₂R^{c}, —C(O)NR^{a}SO₂R^{c}, —C(O)NR^{a}CN, —SO₂N(R^{a}R^{b}), —NR^{a}SO₂R^{b}, —NR^{c}C(O)R^{a}, —NR^{c}C(O)OR^{a}, —NR^{c}C(O)N(R^{a}R^{b}), —C(NR^{c})—N(R^{a}R^{b}), —NR^{d}—C(NR^{c})—N(R^{a}R^{b}), —NR^{a}N(R^{a}R^{b}), —CR^{c}═CR^{a}R^{b}, —C≡CR^{a}, ═O, ═S, ═CR^{a}R^{b}, ═NR^{a}, ═NOR^{a}, or ═NNR^{a}, or two optionally substitutable carbons are linked with C₁₋₃ alkylenedioxy;

each optionally substitutable nitrogen is:

optionally substituted with —CN, —NO₂, —R^{a}, —OR^{a}, —C(O)R^{a}, —C(O)R^{a}-aryl, —OC(O)R^{a}, —C(O)OR^{a}, —SR^{a}, —S(O)R^{a}, —SO₂R^{a}, —SO₃R^{a}, —N(R^{a}R^{b}), —C(O)N(R^{a}R^{b}), —C(O)NR^{a}NR^{b}SO₂R^{c}, —C(O)NR^{a}SO₂R^{c}, —C(O)NR^{a}CN, —SO₂N(R^{a}R^{b}), —NR^{a}SO₂R^{b}, —NR^{c}C(O)R^{a}, —NR^{c}C(O)OR^{a}, —NR^{c}C(O)N(R^{a}R^{b}), or oxygen to form an N-oxide; and

is optionally protonated or quaternary substituted with a nitrogen substituent, thereby carrying a positive charge which is balanced by a pharmaceutically acceptable counterion; and

wherein each of R^{a}, R^{b}, R^{c} and R^{d} is independently —H, alkyl, haloalkyl, aralkyl, aryl, heteroaryl, heterocyclyl, or cycloaliphatic, or

in any occurrence of —N(R^{a}R^{b}), R^{a} and R^{b} taken together with the nitrogen to which they are attached optionally form an optionally substituted heterocyclic group.

In some embodiments, when X² and X⁴ are both N and are linked together, they are linked together through an optionally substituted alkyl, alkenyl, heteroalkyl, or heteroalkenyl linking group, thereby forming an optionally substituted 6-7 membered heteroaryl or heterocyclyl ring.

In some embodiments, each optionally substitutable carbon is optionally substituted with a substituent other than —SR^{a}.

In some embodiments, ring A when monosubstituted phenyl is other than 2-trifluoromethylphenyl, 3-methoxyphenyl, 3-nitrophenyl, 3-trifluoromethylphenyl, 3-vinylphenyl, 4-t-butylphenyl, 4-chlorophenyl, 4-fluorophenyl, 4-methoxyphenyl, 4-methylphenyl, 4-nitrophenyl, 4-trifluoromethylphenyl, and/or 4-vinylphenyl. In some embodiments, ring A when disubstituted phenyl is other than 3,4-dichlorophenyl, 3,5-ditrifluoromethylphenyl, and/or 2-hydroxy-5-nitrophenyl. In some embodiments, these provisos apply when X¹ is NH, X² is NH, X³ is NH, X⁴ is NH, X⁵ is NH₂, and Y is CH₂.

In some embodiments, ring A when substituted phenyl is other than 2-haloalkylphenyl, 3-alkoxyphenyl, 3-nitrophenyl, 3-haloalkylphenyl, 3-vinylphenyl, 4-alkenylphenyl, 4-alkylphenyl, 4-haloalkylphenyl, 4-halophenyl, 4-alkoxyphenyl, and/or 4-nitrophenyl. In some embodiments, ring A when disubstituted phenyl is other than 3,4-dihalophenyl, 3,5-haloalkylphenyl, and/or 2-hydroxy-5-nitrophenyl. In some embodiments, these provisos apply when X¹ is NH, X² is NH, X³ is NH, X⁴ is NH, X⁵ is NH₂, and Y is CH₂.

In some embodiments, ring A is monosubstituted phenyl. In some embodiments, ring A is 2- or 3- or 4-monosubstituted phenyl. In other embodiments, ring A is other than monosubstituted phenyl, or other than 2- or 3- or 4-monosubstituted phenyl. In some such embodiments, X¹ is NH, X² is NH, X³ is NH, X⁴ is NH, X⁵ is NH₂, and Y is CH₂.

In some embodiments, ring A is disubstituted phenyl. In some embodiments, ring A is 2,3- or 2,4- or 2,5- or 2,6- or 3,4- or 3,5-disubstituted phenyl. In other embodiments, ring A is other than disubstituted phenyl, or other than 2,3- or 2,4- or 2,5- or 2,6- or 3,4- or 3,5-disubstituted phenyl. In some such embodiments, X¹ is NH, X² is NH, X³ is NH, X⁴ is NH, X⁵ is NH₂, and Y is CH₂.

In some embodiments, ring A is trisubstituted phenyl. In some embodiments, ring A is 2,3,4- or 2,3,5- or 2,3,6- or 2,4,5- or 2,4,6- or 3,4,5-trisubstituted phenyl. In other embodiments, ring A is other than trisubstituted phenyl, or other than 2,3,4- or 2,3,5- or 2,3,6- or 2,4,5- or 2,4,6- or 3,4,5-trisubstituted phenyl. In some such embodiments, X¹ is NH, X² is NH, X³ is NH, X⁴ is NH, X⁵ is NH₂, and Y is CH₂.

In some embodiments, ring A is tetrasubstituted phenyl. In some embodiments, ring A is 2,3,4,5- or 2,3,4,6- or 2,3,5,6-tetrasubstituted phenyl. In other embodiments, ring A is other than tetrasubstituted phenyl, or other than 2,3,4,5- or 2,3,4,6- or 2,3,5,6-tetrasubstituted phenyl. In some such embodiments, X¹ is NH, X² is NH, X³ is NH, X⁴ is NH, X⁵ is NH₂, and Y is CH₂.

In some embodiments, ring A is pentasubstituted phenyl. In some embodiments, ring A is other than substituted phenyl.

In some embodiments, X¹ is —O—, —S—, or optionally substituted —NH—; X³ is —O—, —S—, optionally substituted —NH— or optionally substituted methylene; X² is S or optionally substituted NH; and X⁴ is S or optionally substituted NH.

In some embodiments R^{a} is other than —H, is other than alkyl, is other than haloalkyl, is other than aralkyl, is other than aryl, is other than heteroaryl, is other than heterocyclyl, or is other than cycloaliphatic.

In some embodiments R^{b} is other than —H, is other than alkyl, is other than haloalkyl, is other than aralkyl, is other than aryl, is other than heteroaryl, is other than heterocyclyl, or is other than cycloaliphatic. In some embodiments, R^{a} is other than heterocyclic.

In some embodiments R^{c} is other than —H, is other than alkyl, is other than haloalkyl, is other than aralkyl, is other than aryl, is other than heteroaryl, is other than heterocyclyl, or is other than cycloaliphatic.

In some embodiments R^{d} is other than —H, is other than alkyl, is other than haloalkyl, is other than aralkyl, is other than aryl, is other than heteroaryl, is other than heterocyclyl, or is other than cycloaliphatic.

In some embodiments, Group A is phenyl substituted in at least the 2-position. In some such embodiments, the phenyl is substituted in the 2-position with halogen. In some such embodiments, the phenyl is substituted in the 2-position with a substituent other than haloalkyl, for example trifluoromethyl. In some such embodiments, the phenyl is substituted in the 2-position with a substituent other than OH. In some such embodiments, the phenyl is substituted in the 2-position with a substituent other than SR^{a}.

In some embodiments, Group A is phenyl substituted in at least the 2-position. In some such embodiments, the phenyl is substituted in the 2-position with a substituent other than haloalkyl, for example trifluoromethyl. In some such embodiments, the phenyl is substituted in the 2-position with a substituent other than OH. In some such embodiments, the phenyl is substituted in the 2-position with a substituent other than SR^{a}.

In some embodiments, Group A is phenyl substituted in at least the 4-position. In some such embodiments, the phenyl is substituted in the 4-position with a substituent other than nitro. In some such embodiments, the phenyl is substituted in the 4-position with a substituent other than halogen. In some such embodiments, the phenyl is substituted in the 4-position with a substituent other than halogen unless the ring is further substituted; in some such embodiments the further substituent, if in the 3-position, is other than halogen. In some such embodiments, the phenyl is substituted in the 4-position with a substituent other than SR^{a}. In some such embodiments, the phenyl is substituted in the 2-position with a substituent other than SR^{a}.

In some embodiments, the Group A is substituted phenyl or optionally substituted naphthyl or pyridyl. In some embodiments, in Group A, an unsubstituted ring atom is adjacent to the ring atom attached to Y.

In some embodiments, Y is C(O), C(S), or methylene optionally substituted with hydroxyl, C₁₋₆ alkyl, C₁₋₆ alkoxy, C₁₋₆ haloalkyl, C₁₋₆ haloalkoxy, C₁₋₆ alkyl substituted with aryl, aryl, heteroaryl, heterocyclyl, or cycloaliphatic. In some embodiments, Y is C(O), or methylene optionally substituted with hydroxyl, C₁₋₆ alkyl, C₁₋₆ alkoxy, C₁₋₆ haloalkyl, C₁₋₆ haloalkoxy, or C₁₋₆ alkyl substituted with aryl. In some embodiments, Y is methylene optionally substituted with hydroxyl, C₁₋₆ alkyl, Cₜ₋₆ alkoxy, or C₁₋₆ alkyl substituted with aryl. In some embodiments, Y is methylene optionally substituted with C₁₋₃ alkyl. In some embodiments, Y is methylene.

In some embodiments, the compound is represented by the following structural formula (Ia):

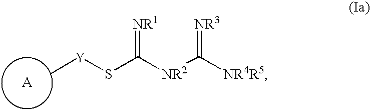

or a salt such as a pharmaceutically acceptable salt thereof, wherein:

R¹ is hydrogen, hydroxyl, C₁₋₆ alkyl, C₁₋₆ alkoxy, C₁₋₆ haloalkyl, C₁₋₆ haloalkoxy, C₁₋₆ alkyl substituted with aryl, aryl, heteroaryl, heterocyclyl, or cycloaliphatic;

R² is hydrogen, hydroxyl, C₁₋₆ alkyl, C₁₋₆ alkoxy, C₁₋₆ haloalkyl, C₁₋₆ haloalkoxy, C₁₋₆ alkyl substituted with aryl, aryl, heteroaryl, heterocyclyl, or cycloaliphatic;

R³ is hydrogen, hydroxyl, C₁₋₆ alkyl, C₁₋₆ alkoxy, C₁₋₆ haloalkyl, C₁₋₆ haloalkoxy, C₁₋₆ alkyl substituted with aryl, aryl, heteroaryl, heterocyclyl, or cycloaliphatic;

R⁴ is hydrogen, hydroxyl, C₁₋₆ alkyl, C₁₋₆ alkoxy, C₁₋₆ haloalkyl, C₁₋₆ haloalkoxy, C₁₋₆ alkyl substituted with aryl, aryl, heteroaryl, heterocyclyl, or cycloaliphatic; and

R⁵ is hydrogen, hydroxyl, C₁₋₆ alkyl, C₁₋₆ alkoxy, C₁₋₆ haloalkyl, C₁₋₆ haloalkoxy, C₁₋₆ alkyl substituted with aryl, aryl, heteroaryl, heterocyclyl, or cycloaliphatic. In some embodiments, R¹ is hydrogen, hydroxyl, C₁₋₆ alkyl, C₁₋₆ alkoxy, C₁₋₆ haloalkyl, C₁₋₆ haloalkoxy, or C₁₋₆ alkyl substituted with aryl. In some embodiments, R¹ is hydrogen, hydroxyl, C₁₋₆ alkyl, C₁₋₆ alkoxy, or C₁₋₆ alkyl substituted with aryl. In some embodiments, R¹ is hydrogen or C₁₋₃ alkyl, for example methyl. In some embodiments, R¹ is hydrogen.

In some embodiments, R² is hydrogen, hydroxyl, C₁₋₆ alkyl, C₁₋₆ alkoxy, C₁₋₆ haloalkyl, C₁₋₆ haloalkoxy, or C₁₋₆ alkyl substituted with aryl. In some embodiments, R² is hydrogen, hydroxyl, C₁₋₆ alkyl, C₁₋₆ alkoxy, or C₁₋₆ alkyl substituted with aryl. In some embodiments, R² is hydrogen or C₁₋₃ alkyl, for example methyl. In some embodiments, R² is hydrogen.

In some embodiments, R³ is hydrogen, hydroxyl, C₁₋₆ alkyl, C₁₋₆ alkoxy, C₁₋₆ haloalkyl, C₁₋₆ haloalkoxy, or C₁₋₆ alkyl substituted with aryl. In some embodiments, R³ is hydrogen, hydroxyl, C₁₋₆ alkyl, C₁₋₆ alkoxy, or C₁₋₆ alkyl substituted with aryl. In some embodiments, R³ is hydrogen or C₁₋₃ alkyl, for example methyl. In some embodiments, R³ is hydrogen.

In some embodiments, R⁴ is hydrogen, hydroxyl, C₁₋₆ alkyl, C₁₋₆ alkoxy, C₁₋₆ haloalkyl, C₁₋₆ haloalkoxy, or C₁₋₆ alkyl substituted with aryl. In some embodiments, R⁴ is hydrogen, hydroxyl, C₁₋₆ alkyl, C₁₋₆ alkoxy, or C₁₋₆ alkyl substituted with aryl. In some embodiments, R⁴ is hydrogen or C₁₋₃ alkyl, for example methyl. In some embodiments, R⁴ is hydrogen.

In some embodiments, R⁵ is hydrogen, hydroxyl, C₁₋₆ alkyl, C₁₋₆ alkoxy, C₁₋₆ haloalkyl, C₁₋₆ haloalkoxy, or C₁₋₆ alkyl substituted with aryl. In some embodiments, R⁵ is hydrogen, hydroxyl, C₁₋₆ alkyl, C₁₋₆ alkoxy, or C₁₋₆ alkyl substituted with aryl. In some embodiments, R⁵ is hydrogen or C₁₋₃ alkyl, for example methyl. In some embodiments, R⁵ is hydrogen.

In some embodiments of the compounds of formula Ia, A is substituted phenyl. In particular embodiments thereof, Y is methylene, R¹, R², R³, R⁴ and R⁵ are hydrogen

In some embodiments of the compounds of formula Ia, A is optionally substituted naphthyl, for example optionally substituted 1-naphthyl or 2-naphthyl. In particular embodiments thereof, Y is methylene, R¹, R², R³, R⁴ and R⁵ are hydrogen.

In the compounds of formula I, and Ia, and the embodiments thereof, in some embodiments, Group A is substituted with a substitutent selected from —F, —Cl, —Br, —I, —CN, —NO₂, —R^{a}, —OR^{a}, —C(O)R^{a}, —OC(O)R^{a}, —C(O)OR^{a}, —SR^{a}, —SO₂R^{a}, —SO₃R^{a}, —OSO₂R^{a}, —OSO₃R^{a}, —N(R^{a}R^{b}), —C(O)N(R^{a}R^{b}), —C(O)NR^{a}NR^{b}SO₂R^{c}, —C(O)NR^{a}SO₂R^{c}, —SO₂N(R^{a}R^{b}), —NR^{a}SO₂R^{b}, —NR^{c}C(O)R^{a}, and —NR^{c}C(O)OR^{a}, or two substitutable carbons are linked with C₁₋₃ alkylenedioxy. For example, in some embodiments, one, two or three substitutable carbons in Group A may be substituted with a substituent independently selected from —F, —Cl, —Br, —I, —CN, —NO₂, Cl₁₋₆ alkyl, C₁₋₆ alkoxy, —CF₃, and C₁₋₆ haloalkoxy, or two substitutable carbons may be linked with C₁₋₂ alkylenedioxy.

In some embodiments, Group A is phenyl, wherein one, two or three substitutable carbons of the phenyl are substituted with a substituent independently selected from —F, —Cl, —Br, —I, —CN, —NO₂, C₁₋₆ alkyl, C₁₋₆ alkoxy, —CF₃, and C₁₋₆ haloalkoxy, or two substitutable carbons are linked with C₁₋₂ alkylenedioxy. In some embodiments, Group A is phenyl unsubstituted at its 6-position. In some embodiments, Group A is 2,4-substituted phenyl. In some embodiments, Group A is 2,4-disubstituted phenyl substituted at least the 2-position, or in at least the 4-position, or in both the 2- and 4-positions with halogen; in some such embodiments, one of the halogens may be chlorine. In some embodiments, Group A or is phenyl monosubstituted at its 2, 3, or 4 positions or independently disubstituted at its 2,3, 2,4, 2,5 or 3,4 positions with —F, —Cl, —Br, —NO₂, C₁₋₆ alkyl, or —CF₃. In some embodiments, Group A is phenyl independently disubstituted at its 2,3, 2,4, 3,4, or 2,5 positions with —NO₂, —Cl, —F or —CF₃. In some embodiments, Group A is phenyl monosubstituted at its 2, 3, or 4 position with —NO₂, —Cl or —F. In some embodiments, Group A is phenyl independently disubstituted at its 2,4 positions with —NO₂, —Cl or —F.

In some embodiments, Group A is unsubstituted 2-naphthyl or 1-substituted 2-naphthyl. In some embodiments, Group A is naphthyl optionally substituted with one or more of —F, —Cl, —Br, —NO₂, C₁₋₆ alkyl, or —CF₃. In some embodiments, Group A is naphthyl optionally monosubstituted with —F, —Cl, —Br, —NO₂, or —CF₃. In some embodiments, Group A is naphthyl optionally monosubstituted with —F, —Cl, or —Br.

Particular compounds of interest include the following compounds and salts such as pharmaceutically acceptable salts thereof, particularly the 2,4-dichlorophenyl compound.

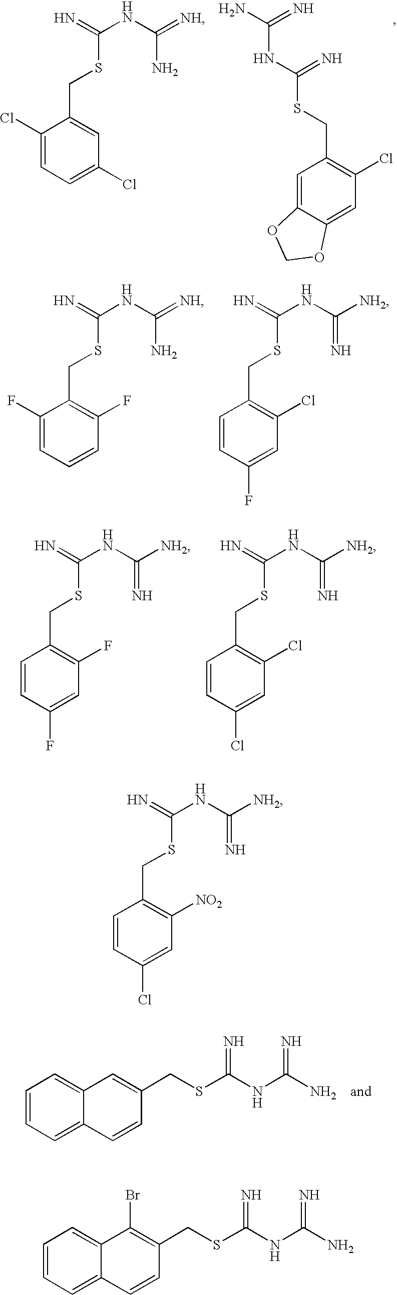

In another aspect, there is provided a compound according to formula (II):

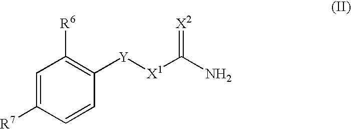

or a salt such as a pharmaceutically acceptable salt thereof, wherein:

Y is optionally substituted methylene;

X¹ is —O—, —S—, or optionally substituted —NH—; and

X² is S or optionally substituted NH;

R⁶ and R⁷ are independently —F, —Cl, —Br, —I, —NO₂, —CN, —CF₃, or C₁-C₆ alkoxy;

wherein

each optionally substitutable carbon is optionally substituted with —F, —Cl, —Br, —I, —CN, —NO₂, —R^{a}, —OR^{a}, —C(O)R^{a}, —OC(O)R^{a}, —C(O)OR^{a}, —C(S)R^{a}, —OC(S)R^{a}, —C(S)OR^{a}, —C(O)SR^{a}, —C(S)SR^{a}, —S(O)R^{a}, —SO₂R^{a}, —SO₃R^{a}, —OSO₂R^{a}, —OSO₃R^{a}, —PO₂R^{a}R^{b}, —OPO₂R^{a}R^{b}, —PO₃R^{a}R^{b}, —OPO₃R^{a}R^{b}, —N(R^{a}R^{b}), —C(O)N(R^{a}R^{b}), —C(O)NR^{a}NR^{b}SO₂R^{c}, —C(O)NR^{a}SO₂R^{c}, —C(O)NR^{a}CN, —SO₂N(R^{a}R^{b}), —NR^{a}SO₂R^{b}, —NR^{c}C(O)R^{a}, —NR^{c}C(O)OR^{a}, —NR^{c}C(O)N(R^{a}R^{b}), —C(NR^{c})—N(R^{a}R^{b}), —NR^{d}—C(NR^{c})—N(R^{a}R^{b}), —NR^{a}N(R^{a}R^{b}), —CR^{c}═CR^{a}R^{b}, —C≡CR^{a}, ═O, ═S, ═CR^{a}R^{b}, ═NR^{a}, ═NOR^{a}, or ═NNR^{a}, or two optionally substitutable carbons are linked with C₁₋₃ alkylenedioxy;

each optionally substitutable nitrogen is:

optionally substituted with —CN, —NO₂, —R^{a}, —OR^{a}, —C(O)R^{a}, —C(O)R^{a}-aryl, —OC(O)R^{a}, —C(O)OR^{a}, —S(O)R^{a}, —SO₂R^{a}, —SO₃R^{a}, —N(R^{a}R^{b}), —C(O)N(R^{a}R^{b}), —C(O)NR^{a}NR^{b}SO₂R^{c}, —C(O)NR^{a}SO₂R^{c}, —C(O)NR^{a}CN, —SO₂N(R^{a}R^{b}), —NR^{a}SO₂R^{b}, —NR^{c}C(O)R^{a}, —NR^{c}C(O)OR^{a}, —NR^{c}C(O)N(R^{a}R^{b}), or oxygen to form an N-oxide; and

optionally is protonated or quaternary substituted with a nitrogen substituent, thereby carrying a positive charge which is balanced by a pharmaceutically acceptable counterion; and

wherein each of R^{a}, R^{b}, R^{c} and R^{d} is independently —H, alkyl, haloalkyl, aralkyl, aryl, heteroaryl, heterocyclyl, or cycloaliphatic, or

in any occurrence of —N(R^{a}R^{b}), R^{a} and R^{b} taken together with the nitrogen to which they are attached optionally form an optionally substituted heterocyclic group.

In some embodiments of the compounds of formula II, R⁶ and R⁷ are not both —Cl and R⁶ and R⁷ are not both —CF₃.

In some embodiments of the compounds of formula II, R⁶ and R⁷ are not both —F, R⁶ and R⁷ are not both —Br, R⁶ and R⁷ are not both —I, R⁶ and R⁷ are not both —NO₂, and R⁶ and R⁷ are not both —CH₃. In some embodiments, this proviso applies when Y is —CH₂—, X¹ is S and X² is NH.

In some embodiments of the compounds of formula II, Y is C(O), C(S), or methylene optionally substituted with hydroxyl, C₁₋₆ alkyl, C₁₋₆ alkoxy, C₁₋₆ haloalkyl, C₁₋₆ haloalkoxy, C₁₋₆ alkyl substituted with aryl, aryl, heteroaryl, heterocyclyl, or cycloaliphatic. In some embodiments, Y is methylene optionally substituted with hydroxyl, C₁₋₆ alkyl, C₁₋₆ alkoxy, or C₁₋₆ alkyl substituted with aryl. In some embodiments, Y is methylene optionally substituted with C₁₋₃ alkyl, for example methyl. In some embodiments, Y is methylene.

In some embodiments, the compound of formula II is represented by the following structural formula:

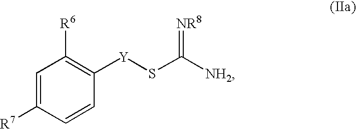

or a salt such as a pharmaceutically acceptable salt thereof, wherein R⁸ is hydrogen, hydroxyl, C₁₋₆ alkyl, C₁₋₆ alkoxy, C₁₋₆ haloalkyl, C₁₋₆ haloalkoxy, C₁₋₆ alkyl substituted with aryl, aryl, heteroaryl, heterocyclyl, or cycloaliphatic. In some embodiments thereof, is hydrogen, hydroxyl, C₁₋₆ alkyl, C₁₋₆ alkoxy, or C₁₋₆ alkyl substituted with aryl. In some embodiments, R⁸ is hydrogen or C₁₋₃ alkyl, for example methyl. In some embodiments, R⁸ is hydrogen.

In the preferred embodiments, Y is methylene and R⁸ is hydrogen.

In some embodiments, R⁶ and R⁷ are independently —F, —Cl, —Br, —NO₂, or —CF₃.

Compounds according to formula II of particular interest include those wherein the compound is selected from the group consisting of:

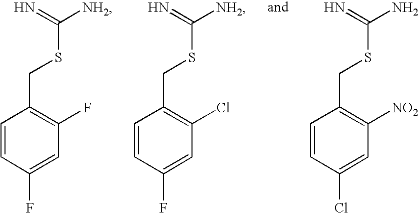

and salts such as pharmaceutically acceptable salts thereof.

In another aspect, compounds are included which are represented by one of the following structural formulae (Ib) and (IIb):

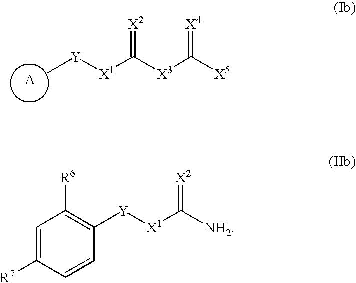

wherein

Group A is substituted phenyl, optionally substituted 6-membered heteroaryl, or optionally substituted fused bicyclic 9-10 membered aryl or heteroaryl;

Y is optionally substituted methylene;

X¹ and X³ are independently —O—, —S—, or optionally substituted —NH—, or X³ is optionally substituted methylene;

X² and X⁴ are independently S or optionally substituted NH, or X² and X⁴ are both N and are linked together through a bond or an optionally substituted alkyl, alkenyl, heteroalkyl, or heteroalkenyl linking group, thereby forming an optionally substituted 5-7 membered heteroaryl or heterocyclyl ring;

X⁵ is an optionally substituted —NH₂ or 3-7 membered heteroaryl or heterocyclyl ring;

R⁶ and R⁷ are independently —F, —Cl, —Br, —I, —NO₂, —CN, —CF₃, or C₁-C₆ alkoxy, provided that R⁶ and R⁷ are not both —Cl and R₁ and R₂ are not both —CF₃. In some embodiments, R⁶ and R⁷ are not both —F. In certain embodiments, R⁶ and R⁷ are independently —F, —Cl, —Br, —NO₂, or —CF₃, or in particular embodiments, R⁶ and R⁷ are independently —F, or —NO₂;

each substitutable carbon atom (e.g., each optionally substituted carbon) is optionally substituted with —F, —Cl, —Br, —I, —CN, —NO₂, —R^{a}, —OR^{a}, —C(O)R^{a}, —OC(O)R^{a}, —C(O)OR^{a}, —SR^{a}, —C(S)R^{a}, —OC(S)R^{a}, —C(S)OR^{a}, —C(O)SR^{a}, —C(S)SR^{a}, —S(O)R^{a}, —SO₂R^{a}, —SO₃R^{a}, —OSO₂R^{a}, —OSO₃R^{a}, —PO₂R^{a}R^{b}, —OPO₂R^{a}R^{b}, —PO₃R^{a}R^{b}, —OPO₃R^{a}R^{b}, —N(R^{a}R^{b}), —C(O)N(R^{a}R^{b}), —C(O)NR^{a}NR^{b}SO₂R^{c}, —C(O)NR^{a}SO₂R^{c}, —C(O)NR^{a}CN, —SO₂N(R^{a}R^{b}), —NR^{a}SO₂R^{b}, —NR^{c}C(O)R^{a}, —NR^{c}C(O)OR^{a}, —NR^{c}C(O)N(R^{a}R^{b}), —C(NR^{c})—N(R^{a}R^{b}), —NR^{d}—C(NR^{c})—N(R^{a}R^{b}), —NR^{a}N(R^{a}R^{b}), —CR^{c}═CR^{a}R^{b}, —C≡CR^{a}, ═O, —CR^{a}R^{b}, ═NR^{a}, —NOR^{a}, or ═NNR^{a}, or two substitutable carbons are linked with C₁₋₃ alkylenedioxy;

each substitutable nitrogen (e.g., each optionally substituted nitrogen) is optionally substituted with —CN, —NO₂, —R^{a}, —OR^{a}, —C(O)R^{a}, —C(O)R^{a}-aryl, —OC(O)R^{a}, —C(O)OR^{a}, —SR^{a}, S(O)R^{a}, —SO₂R^{a}, —SO₃R^{a}, —N(R^{a}R^{b}), —C(O)N(R^{a}R^{b}), —C(O)NR^{a}NR^{b}SO₂R^{c}, —C(O)NR^{a}SO₂R^{c}, —C(O)NR^{a}CN, —SO₂N(R^{a}R^{b}), —NR^{a}SO₂R^{b}, —NR^{c}C(O)R^{a}, —NR^{c}C(O)OR^{a}, —NR^{c}C(O)N(R^{a}R^{b}), or oxygen to form an N-oxide and each nitrogen can also be optionally protonated or quaternary substituted with a nitrogen substituent, thereby carrying a positive charge which is balanced by a pharmaceutically acceptable counterion; and

Each R^{a}-R^{d} is independently —H, alkyl, alkoxy, haloalkyl, haloalkoxy, aralkyl, aryl, heteroaryl, heterocyclyl, or cycloaliphatic, or, —N(R^{a}R^{b}), taken together, is an optionally substituted heterocyclic group.

In various embodiments of the compounds Ib and IIa, Y is C(O), C(S), or methylene optionally substituted with hydroxyl, C₁₋₆ alkyl, C₁₋₆ alkoxy, C₁₋₆ haloalkyl, C₁₋₆ haloalkoxy, C₁₋₆ alkyl substituted with aryl, aryl, heteroaryl, heterocyclyl, or cycloaliphatic. In some embodiments, Y is C(O), or methylene optionally substituted with hydroxyl, C₁₋₆ alkyl, C₁₋₆ alkoxy, C₁₋₆ haloalkyl, C₁₋₆ haloalkoxy, or C₁₋₆ alkyl substituted with aryl. In certain embodiments, Y is methylene optionally substituted with hydroxyl, C₁₋₆ alkyl, C₁₋₆ alkoxy, or C₁₋₆ alkyl substituted with aryl. In particular embodiments, Y is methylene optionally substituted with C₁₋₃ alkyl.

In the compounds of formula IIb, Group A can be substituted phenyl or optionally substituted naphthyl or pyridyl. In some embodiments, in Group A, an unsubstituted ring atom is adjacent to the ring atom attached to Y. For example, when Group A is a phenyl, the 6-position of that phenyl can be unsubstituted.

In some embodiments, the compound according to formula Ib is represented by the following structural formula (Ic):

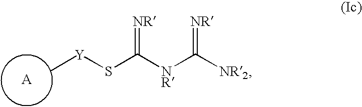

wherein each R′ is independently hydrogen, hydroxyl, C₁₋₆ alkyl, C₁₋₆ alkoxy, C₁₋₆ haloalkyl, C₁₋₆ haloalkoxy, C₁₋₆ alkyl substituted with aryl, aryl, heteroaryl, heterocyclyl, or cycloaliphatic. In some embodiments, each R′ is independently hydrogen, hydroxyl, C₁₋₆ alkyl, C₁₋₆ alkoxy, C₁₋₆ haloalkyl, C₁₋₆ haloalkoxy, or C₁₋₆ alkyl substituted with aryl. In certain embodiments, each R′ is independently hydrogen, hydroxyl, C₁₋₆ alkyl, C₁₋₆ alkoxy, or C₁₋₆ alkyl substituted with aryl. In particular embodiments, each R′ is independently hydrogen or C₁₋₃ alkyl.

In various embodiments, the compound according to the formula Ib may be represented by one of the following structural formulae:

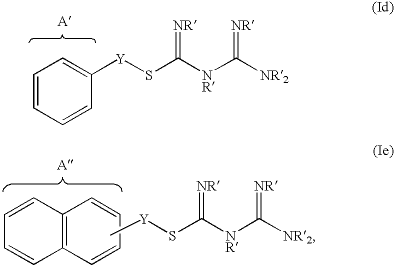

wherein A′ is substituted phenyl and A″ is optionally substituted naphthyl. In some embodiments, the compound can be represented by the following structural formula:

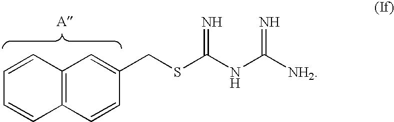

In some embodiments, the compound can be represented by the following structural formula:

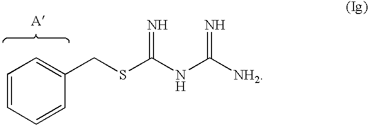

In various embodiments of the compounds of formula Ib, one or more substitutable carbons in Group A, Ring A′ or Ring A″ is substituted with —F, —Cl, —Br, —I, —CN, —NO₂, —R^{a}, —OR^{a}, —C(O)R^{a}, —OC(O)R^{a}, —C(O)OR^{a}, —SR^{a}, —SO₂R^{a}, —SO₃R^{a}, —OSO₂R^{a}, —OSO₃R^{a}, —N(R^{a}R^{b}), —C(O)N(R^{a}R^{b}), —C(O)NR^{a}NR^{b}SO₂R^{c}, —C(O)NR^{a}SO₂R^{c}, —SO₂N(R^{a}R^{b}), —NR^{a}SO₂R^{b}, —NR^{c}(O)R^{a}, or —NR^{c}C(O)OR^{a}, or two substitutable carbons are linked with C₁₋₃ alkylenedioxy. In some embodiments, in Group A, Ring A′ or Ring A″: one, two or three substitutable carbons are substituted with —F, —Cl, —Br, —I, —CN, —NO₂, C₁₋₆ alkyl, C₁₋₆ alkoxy, —CF₃, or C₁₋₆ haloalkoxy, or two substitutable carbons are linked with C₁₋₂ alkylenedioxy.

In various embodiments of the compounds of formula Ib, Group A or Ring A′ is phenyl unsubstituted at its 6-position. In some embodiments, Group A or Ring A′ is 2,4-substituted phenyl. In certain embodiments, Group A or Ring A′ is phenyl monosubstituted at its 2, 3, or 4 positions or independently disubstituted at its 2,3, 2,4, 2,5 or 3,4 positions with —F, —Cl, —Br, —NO₂, C₁₋₆ alkyl, or —CF₃. In particular embodiments, Group A or Ring A′ is phenyl independently disubstituted at its 2,3, 2,4, 3,4, or 2,5 positions with —NO₂, —Cl, —F or —CF₃. In some embodiments, Group A or Ring A′ is phenyl monosubstituted at its 2, 3, or 4 position with NO₂, —Cl or —F. In certain embodiments, Group A or Ring A′ is phenyl independently disubstituted at its 2,4 positions with NO₂, —Cl or —F.

In various embodiments, Group A or Ring A″ is unsubstituted 2-naphthyl or 1-substituted 2-naphthyl. In some embodiments, Group A or Ring A″ is naphthyl optionally substituted with one or more of —F, —Cl, —Br, —NO₂, C₁₋₆ alkyl, or —CF₃. In certain embodiments, Group A or Ring A″ is naphthyl optionally monosubstituted with —F, —Cl, —Br, —NO₂, or —CF₃. In particular embodiments, Group A or Ring A″ is naphthyl optionally monosubstituted with —F, —Cl, or —Br.

In various embodiments, the compound is represented by the following structural formula:

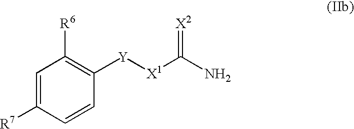

Y can be as defined in any embodiment herein above. In some embodiments, Y is C(O), C(S), or methylene optionally substituted with hydroxyl, C₁₋₆ alkyl, C₁₋₆ alkoxy, C₁₋₆ haloalkyl, C₁₋₆ haloalkoxy, C₁₋₆ alkyl substituted with aryl, aryl, heteroaryl, heterocyclyl, or cycloaliphatic. In certain embodiments, Y is methylene optionally substituted with hydroxyl, C₁₋₆ alkyl, C₁₋₆ alkoxy, or C₁₋₆ alkyl substituted with aryl. In particular embodiments, Y is methylene optionally substituted with C₁₋₃ alkyl.

In various embodiments, the compound is represented by the following structural formula:

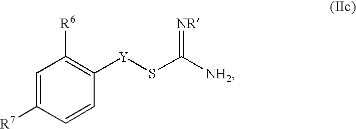

R′ can be as defined in any embodiment herein above. In some embodiments, R′ is hydrogen, hydroxyl, C₁₋₆ alkyl, C₁₋₆ alkoxy, C₁₋₆ haloalkyl, C₁₋₆ haloalkoxy, C₁₋₆ alkyl substituted with aryl, aryl, heteroaryl, heterocyclyl, or cycloaliphatic. In certain embodiments, R′ is hydrogen, hydroxyl, C₁₋₆ alkyl, C₁₋₆ alkoxy, or C₁₋₆ alkyl substituted with aryl. In particular embodiments, R′ is hydrogen or C₁₋₃ alkyl, for example methyl. In particular embodiments, R′ is hydrogen.

Also included are pharmaceutically acceptable salts, solvates, hydrates, tautomers, stereoisomers and diasteromers of the compounds. The compounds can be modulators of Rb:Raf 1 interactions.

It is to be understood that other embodiments of the invention will combine the features of embodiments explicitly described above. Embodiments defined by such combinations are contemplated as embodiments of the invention.

### III. SALTS

The compounds described above, and any of the embodiments thereof, as well as intermediates used in making the compounds may take the form of salts. The compounds, compositions and methods of the present invention include salts of the disclosed compounds, particularly pharmaceutically acceptable salts, and methods and compositions using them.

The disclosed compounds can have one or more sufficiently acidic protons that can react with a suitable organic or inorganic base to form a base addition salt. When it is stated that a compound has a hydrogen atom bonded to an oxygen, nitrogen, or sulfur atom, it is contemplated that the compound also includes salts thereof where this hydrogen atom has been reacted with a suitable organic or inorganic base to form a base addition salt. Base addition salts include those derived from inorganic bases, such as ammonium or alkali or alkaline earth metal hydroxides, carbonates, bicarbonates, and the like, and organic bases such as alkoxides, alkyl amides, alkyl and aryl amines, and the like. Such bases useful in preparing the salts of this invention thus include sodium hydroxide, potassium hydroxide, ammonium hydroxide, potassium carbonate, and the like.

The term “salts” embraces addition salts of free acids or free bases which are compounds described herein. The term “pharmaceutically-acceptable salt” refers to salts which possess toxicity profiles within a range that affords utility in pharmaceutical applications, such that the salt is suitable for administration to a subject. Pharmaceutically unacceptable salts may nonetheless possess properties such as high crystallinity, which may render them useful, for example in processes of synthesis, purification or formulation of compounds described herein. In general the useful properties of the compounds described herein do not depend critically on whether the compound is or is not in a salt form, so unless clearly indicated otherwise (such as specifying that the compound should be in “free base” or “free acid” form), reference in the specification to a compound should generally be understood as encompassing salts of the compound, whether or not this is explicitly stated.

When the disclosed compounds contain a basic group, such as an amine, suitable pharmaceutically-acceptable acid addition salts may be prepared from an inorganic acid or from an organic acid. Examples of inorganic acids include hydrochloric, hydrobromic, hydriodic, carbonic, sulfuric, phosphoric and nitric acids. Appropriate organic acids may be selected from aliphatic, cycloaliphatic, aromatic, araliphatic, heterocyclic, carboxylic and sulfonic classes of organic acids, examples of which include p-toluenesulfonic, methanesulfonic, oxalic, p-bromophenyl-sulfonic, carbonic, succinic, citric, benzoic, acetic acid, formic, acetic, propionic, glycolic, gluconic, lactic, malic, tartaric, ascorbic, glucuronic, maleic, fumaric, pyruvic, aspartic, glutamic, anthranilic, 4-hydroxybenzoic, phenylacetic, mandelic, embonic (pamoic), ethanesulfonic, benzenesulfonic, pantothenic, trifluoromethanesulfonic, 2-hydroxyethanesulfonic, sulfanilic, cyclohexylaminosulfonic, stearic, alginic, β-hydroxybutyric, salicylic, galactaric and galacturonic acid. Examples of such salts include the sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caproate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, sulfonate, xylenesulfonate, phenylacetate, phenylpropionate, phenylbutyrate, citrate, lactate, gamma-hydroxybutyrate, glycolate, tartrate, methanesulfonate, propanesulfonate, naphthalene-1-sulfonate, naphthalene-2-sulfonate, mandelate, and the like. In certain embodiments, the disclosed compound forms a pharmaceutically acceptable salt with HCl, HF, HBr, HI, trifluoracetic acid, or sulfuric acid. In particular embodiments, the disclosed compounds form a pharmaceutically acceptable salt with sulfuric acid. Examples of acids which form pharmaceutically unacceptable acid addition salts include, for example, perchlorates and tetrafluoroborates.

Salts of compounds having an acidic group can be formed by the reaction of the disclosed compounds with a suitable base. For example, salts can be formed by the reaction of the disclosed compounds with one equivalent of a suitable base to form a monovalent salt (i.e., the compound has single negative charge that is balanced by a pharmaceutically acceptable counter cation, e.g., a monovalent cation) or with two equivalents of a suitable base to form a divalent salt (e.g., the compound has a two-electron negative charge that is balanced by two pharmaceutically acceptable counter cations, e.g., two pharmaceutically acceptable monovalent cations or a single pharmaceutically acceptable divalent cation).

Suitable pharmaceutically acceptable base addition salts include, for example, metallic salts including alkali metal, alkaline earth metal and transition metal salts such as, for example, lithium, sodium, potassium, magnesium, calcium and zinc salts. Pharmaceutically acceptable base addition salts also include organic salts made from basic amines such as, for example, N,N′-dibenzylethylenediamine, chloroprocaine, choline, diethanolamine, ethylenediamine, meglumine (N-methylglucamine) and procaine. Salts can also be formed with ammonium compounds, NR₄⁺, wherein each R is independently hydrogen, an optionally substituted aliphatic group (e.g., a hydroxyalkyl group, aminoalkyl group or ammoniumalkyl group) or optionally substituted aryl group, or two R groups, taken together, form an optionally substituted non-aromatic heterocyclic ring optionally fused to an aromatic ring. Generally, the pharmaceutically acceptable cation is Li⁺, Na⁺, K⁺, NH₃(C₂H₅OH)⁺ or N(CH₃)₃(C₂H₅OH)⁺.

Where applicable, any of the salt forms described above can be applied to any of the compounds or embodiments thereof described in the Summary or Section II above. Any of the salt forms appropriate for compounds containing a basic group can be applied to any of the compounds having a basic nitrogen—such as the isothiourea compounds and amidinoisothiourea compounds described above. In particular, the hydrochloride, hydrobromide, sulfatep-toluenesulfonate, methanesulfonae, succinate, citrate, benzoate, lactate, maliate, tartrate, maleate, fumarate, and benzenesulfonate salts of the disclosed compounds may be mentioned.

The salt forms described above as being appropriate for compounds containing a base can particularly be applied as being of interest in Section II above. In particular, each one of the salt forms described above as being appropriate for compounds containing a base can particularly be applied to each one of the following compounds, and, in particular, the hydrochloride, hydrobromide, sulfatep-toluenesulfonate, methanesulfonae, succinate, citrate, benzoate, lactate, maliate, tartrate, maleate, fumarate, and benzenesulfonate salts of the disclosed compounds may be mentioned.

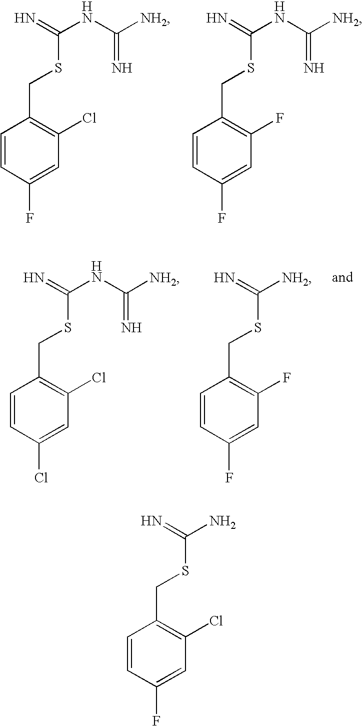

The salt forms suitable for use with containing a base described above are particularly applicable to the 2,4-dichlorophenyl amindinoisothiourea whose structure is provided above.

All of these salts may be prepared by conventional means from the corresponding compound by reacting the compound with the appropriate acid or base. Preferably the salts are in crystalline form, and preferably prepared by crystallization of the salt from a suitable solvent. The person skilled in the art will know how to prepare and select suitable salts for example, as described in Handbook of Pharmaceutical Salts: Properties, Selection, and Use By P. H. Stahl and C. G. Wermuth (Wiley-VCH 2002).

### IV. SOLVATE FORMS

The disclosed compounds, and salts thereof as well as intermediates used in making the compounds may take the form of solvates, including hydrates. Thus, the compounds include solvate forms for the compound, and the compositions and methods disclosed herein, include compositions and methods wherein the disclosed compound is present or used in the form of a solvate or hydrate, preferably a pharmaceutically acceptable solvate or hydrate. The term “solvate” means a compound of the present invention or a salt thereof, that further includes a stoichiometric or non-stoichiometric amount of solvent, e.g., water or organic solvent, bound by non-covalent intermolecular forces; where the solvent is water, the term “hydrate” can be used. In general, the useful properties of the compounds described herein are not believed to depend critically on whether the compound or salt thereof is or is not in the form of a solvate.

### V. STEREOCHEMISTRY, TAUTOMERISM, AND CONFORMATIONAL ISOMERISM

It will also be understood that certain disclosed compounds can be obtained as different stereoisomers (e.g., diastereomers and enantiomers) and tautomers.

The disclosed compounds are intended includes all isomeric forms and racemic mixtures of the disclosed compounds and methods of treating a subject with both pure isomers and mixtures thereof, including racemic mixtures. Stereoisomers can be separated and isolated using any suitable method, such as chromatography.

It will also be understood that certain disclosed compounds can take various tautomeric forms, and the depiction of any compound as a particular tautomer does not preclude other corresponding tautomers of that compound.

### A. Geometrical Isomerism

Certain compounds may possess an olefinic double bond. The stereochemistry of compounds possessing an olefinic double bond is designated using the nomenclature using E and Z designations. The compounds are named according to the Cahn-Ingold-Prelog system, described in the IUPAC 1974 Recommendations, Section E: Stereochemistry, in Nomenclature of Organic Chemistry, John Wiley & Sons, Inc., New York, N.Y., 4^{th} ed., 1992, pp. 127-38, the entire contents of which are incorporated herein by reference.

### B. Optical Isomerism

Certain compounds may contain one or more chiral centers, and may exist in, and may be isolated as pure enantiomeric or diastereomeric forms or as racemic mixtures. The formulae are intended to encompass any possible enantiomers, diastereomers, racemates or mixtures thereof which are biologically active.

The isomers resulting from the presence of a single chiral center comprise a pair of non-superimposable isomers that are called “enantiomers.” Single enantiomers of a pure compound are optically active, i.e., they are capable of rotating the plane of plane polarized light. Single enantiomers are designated according to the Cahn-Ingold-Prelog system.

The formulae encompasses diastereomers as well as their racemic and resolved, diastereomerically and enantiomerically pure forms and salts thereof. Diastereomeric pairs may be resolved by known separation techniques including normal and reverse phase chromatography, and crystallization.

“Isolated optical isomer” means a compound which has been substantially purified from the corresponding optical isomer(s) of the same formula. Preferably, the isolated isomer is at least about 80%, more preferably at least 90% pure, even more preferably at least 98% pure, most preferably at least about 99% pure, by weight.

Isolated optical isomers may be purified from racemic mixtures by well-known chiral separation techniques. According to one such method, a racemic mixture of a compound, or a chiral intermediate in the synthesis thereof, is separated into 99 wt. % pure optical isomers by HPLC using a suitable chiral column, such as a member of the series of DAICEL® CHIRALPAK® family of columns (Daicel Chemical Industries, Ltd., Tokyo, Japan). The column is operated according to the manufacturer's instructions.

### C. Conformational Isomerism

Due to chemical properties such as resonance lending some double bond character to a C—N bond, it is possible that individual conformers of certain compounds described above may be observable and even separable under certain circumstances. The compounds therefore includes any possible stable rotamers which are biologically active.

### D. Tautomerism

Certain of the compounds described above may exist in tautomeric forms, which differ by the location of a hydrogen atom and typically are in rapid equilibrium. In such circumstances, molecular formulae drawn will typically only represent one of the possible tautomers even though equilibration of these tautomeric forms will occur in equilibrium in the compound. Examples include keto-enol tautomerism and amide-imidic acid tautomerism. Tautomerism is frequently also seen in heterocyclic compounds. All tautomeric forms of the compounds are to be understood as being included within the scope of the formulae depicted.

### V. PHARMACEUTICAL COMPOSITIONS AND FORMULATIONS

Also included are pharmaceutical compositions comprising the disclosed compounds. A “pharmaceutical composition” comprises a disclosed compound, typically in conjunction with an acceptable pharmaceutical carrier as part of a pharmaceutical composition for administration to a subject.

The disclosed compounds may be administered in the form of a pharmaceutical composition, in combination with a pharmaceutically acceptable carrier. The active ingredient in such formulations may comprise from 0.1 to 99.99 weight percent. “Pharmaceutically acceptable carrier” means any carrier, diluent or excipient which is compatible with the other ingredients of the formulation and not deleterious to the recipient.

The active agent may be administered with a pharmaceutically acceptable carrier selected on the basis of the selected route of administration and standard pharmaceutical practice. The active agent may be formulated into dosage forms according to standard practices in the field of pharmaceutical preparations. See Alphonso Gennaro, ed., Remington: The Science and Practice of Pharmacy, 20th Edition (2003), Mack Publishing Co., Easton, Pa. Suitable dosage forms may comprise, for example, tablets, capsules, solutions, parenteral solutions, troches, suppositories, suspensions, injection compositions, infusion compositions, topical administration solutions, emulsions, capsules, creams, ointments, tablets, pills, lozenges, suppositories, depot preparations, implanted reservoirs, intravaginal rings, coatings on implantable medical devices (e.g., a stent), impregnation in implantable medical devices, and the like. Suitable pharmaceutical carriers may contain inert ingredients which do not interact with the compound.

For parenteral administration, the active agent may be mixed with a suitable carrier or diluent such as water, for example sterile water, an oil (particularly a vegetable oil), ethanol, saline solution (e.g. physiological saline, bacteriostatic saline (saline containing about 0.9% mg/mL benzyl alcohol), phosphate-buffered saline), Hank's solution, Ringer's-lactate, aqueous dextrose (glucose) and related sugar solutions, glycerol, or a glycol such as propylene glycol or polyethylene glycol. Solutions for parenteral administration preferably contain a water soluble salt of the active agent. Stabilizing agents, antioxidant agents and to preservatives may also be added. Suitable antioxidant agents include sulfite, ascorbic acid, citric acid and its salts, and sodium EDTA. Suitable preservatives include benzalkonium chloride, methyl- or propyl-paraben, and chlorobutanol. The composition for parenteral administration may take the form of an aqueous or non-aqueous solution, dispersion, suspension or emulsion.

For example, a sterile injectable composition such as a sterile injectable aqueous or oleaginous suspension, can be formulated according to techniques known in the art using suitable dispersing or wetting agents (such as, for example, Tween 80) and suspending agents. The sterile injectable preparation can also be a sterile injectable solution or suspension in a non-toxic parenterally acceptable diluent or solvent, for example, as a solution in 1,3-butanediol. Other examples of acceptable vehicles and solvents include mannitol, water, Ringer's solution and isotonic sodium chloride solution. In addition, sterile, fixed oils are conventionally employed as a solvent or suspending medium (e.g., synthetic mono- or diglycerides). Fatty acids, such as oleic acid and its glyceride derivatives can be useful in the preparation of injectables, as well as natural pharmaceutically-acceptable oils, such as olive oil or castor oil, for example in their polyoxyethylated versions. Oil solutions or suspensions can also contain a long-chain alcohol diluent or dispersant, or carboxymethyl cellulose or similar dispersing agents.

A composition for oral administration, for example, can be any orally acceptable dosage form including, but not limited to, capsules, tablets, emulsions and aqueous suspensions, dispersions and solutions. The active agent may be combined with one or more solid inactive ingredients for the preparation of tablets, capsules, pills, powders, granules or other suitable oral dosage forms. For example, the active agent may be combined with at least one excipient such as fillers, binders, humectants, disintegrating agents, solution retarders, absorption accelerators, wetting agents absorbents or lubricating agents. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions or emulsions are administered orally, the active ingredient can be suspended or dissolved in an oily phase combined with emulsifying or suspending agents. If desired, certain sweetening, flavoring, or coloring agents can be added. According to one tablet embodiment, the active agent may be combined with carboxymethylcellulose calcium, magnesium stearate, mannitol and starch, and then formed into tablets by conventional tableting methods. Methods for encapsulating compositions (such as in a coating of hard gelatin or cyclodextran) are known in the art (Baker, et al., “Controlled Release of Biological Active Agents”, John Wiley and Sons, 1986).

A nasal aerosol or inhalation composition can be prepared according to techniques well-known in the art of pharmaceutical formulation and can be prepared as solutions in saline, employing benzyl alcohol or other suitable preservatives, absorption promoters to enhance bioavailability, fluorocarbons, and/or other solubilizing or dispersing agents known in the art.

The specific dose of a compound according required to obtain therapeutic benefit in the methods of treatment described herein will, of course, be determined by the particular circumstances of the individual patient including the size, weight, age and sex of the patient, the nature and stage of the disease being treated, the aggressiveness of the disease disorder, and the route of administration of the compound.

For example, a daily dosage from about 0.05 to about 50 mg/kg/day may be utilized, for example a dosage from about 0.1 to about 10 mg/kg/day. Higher or lower doses are also contemplated as it may be necessary to use dosages outside these ranges in some cases. The daily dosage may be divided, such as being divided equally into two to four times per day daily dosing. The compositions may be formulated in a unit dosage form, each dosage containing from about 1 to about 500 mg, more typically, about 10 to about 100 mg of active agent per unit dosage. The term “unit dosage form” refers to physically discrete units suitable as a unitary dosage for human subjects and other mammals, each unit containing a predetermined quantity of active material calculated to produce the desired therapeutic effect, in association with a suitable pharmaceutical excipient.

The pharmaceutical compositions described herein may also be formulated so as to provide slow or controlled release of the active ingredient therein using, for example, hydropropylmethyl cellulose in varying proportions to provide the desired release profile, other polymer matrices, gels, permeable membranes, osmotic systems, multilayer coatings, microparticles, liposomes and/or microspheres.

In general, a controlled-release preparation is a pharmaceutical composition capable of releasing the active ingredient at the required rate to maintain constant pharmacological activity for a desirable period of time. Such dosage forms provide a supply of a drug to the body during a predetermined period of time and thus maintain drug levels in the therapeutic range for longer periods of time than conventional non-controlled formulations.

U.S. Pat. No. 5,674,533 discloses controlled-release pharmaceutical compositions in liquid dosage forms for the administration of moguisteine, a potent peripheral antitussive. U.S. Pat. No. 5,059,595 describes the controlled-release of active agents by the use of a gastro-resistant tablet for the therapy of organic mental disturbances. U.S. Pat. No. 5,591,767 describes a liquid reservoir transdermal patch for the controlled administration of ketorolac, a non-steroidal anti-inflammatory agent with potent analgesic properties. U.S. Pat. No. 5,120,548 discloses a controlled-release drug delivery device comprised of swellable polymers. U.S. Pat. No. 5,073,543 describes controlled-release formulations containing a trophic factor entrapped by a ganglioside-liposome vehicle. U.S. Pat. No. 5,639,476 discloses a stable solid controlled-release formulation having a coating derived from an aqueous dispersion of a hydrophobic acrylic polymer. Biodegradable microparticles are known for use in controlled-release formulations. U.S. Pat. No. 5,354,566 discloses a controlled-release powder that contains the active ingredient. U.S. Pat. No. 5,733,566 describes the use of polymeric microparticles that release antiparasitic compositions.

The controlled-release of the active ingredient may be stimulated by various inducers, for example pH, temperature, enzymes, water, or other physiological conditions or compounds. Various mechanisms of drug release exist. For example, in one embodiment, the controlled-release component may swell and form porous openings large enough to release the active ingredient after administration to a patient. The term “controlled-release component” means a compound or compounds, such as polymers, polymer matrices, gels, permeable membranes, liposomes and/or microspheres that facilitate the controlled-release of the active ingredient in the pharmaceutical composition. In another embodiment, the controlled-release component is biodegradable, induced by exposure to the aqueous environment, pH, temperature, or enzymes in the body. In another embodiment, sol-gels may be used, wherein the active ingredient is incorporated into a sol-gel matrix that is a solid at room temperature. This matrix is implanted into a patient, preferably a mammal, having a body temperature high enough to induce gel formation of the sol-gel matrix, thereby releasing the active ingredient into the patient.

The components used to formulate the pharmaceutical compositions are of high purity and are substantially free of potentially harmful contaminants (e.g., at least National Food grade, generally at least analytical grade, and more typically at least pharmaceutical grade). Particularly for human consumption, the composition is preferably manufactured or formulated under Good Manufacturing Practice standards as defined in the applicable regulations of the U.S. Food and Drug Administration. For example, suitable formulations may be sterile and/or substantially isotonic and/or in full compliance with all Good Manufacturing Practice regulations of the U.S. Food and Drug Administration.

### VI. MODE OF ADMINISTRATION

Formulation of the compound to be administered will vary according to the route of administration selected, e.g., parenteral, oral, buccal, epicutaneous, inhalational, opthalamic, intraear, intranasal, intravenous, intraarterial, intramuscular, intracardiac, subcutaneous, intraosseous, intracutaneous, intradermal, intraperitoneal, topically, transdermal, transmucosal, intraarticular, intrasynovial, intrasternal, intralesional, intracranial inhalational, insufflation, pulmonary, epidural, intratumoral, intrathecal, vaginal, rectal, or intravitreal administration.

An “effective amount” to be administered is the quantity of compound in which a beneficial outcome is achieved when the compound is administered to a subject or alternatively, the quantity of compound that possess a desired activity in vivo or in vitro. In the case of cell proliferation disorders, a beneficial clinical outcome includes reduction in the extent or severity of the symptoms associated with the disease or disorder and/or an increase in the longevity and/or quality of life of the subject compared with the absence of the treatment. The precise amount of compound administered to a subject will depend on the type and severity of the disease or condition and on the characteristics of the subject, such as general health, age, sex, body weight and tolerance to drugs. It will also depend on the degree, severity and type of disorder. The skilled artisan will be able to determine appropriate dosages depending on these and other factors. The interrelationship of dosages for animals and humans (based on milligrams per meter squared of body surface) is described, for example, in Freireich et al., (1966) Cancer Chemother Rep 50: 219. Body surface area may be approximately determined from height and weight of the patient. See, e.g., Scientific Tables, Geigy Pharmaceuticals, Ardley, N.Y., 1970, 537. An effective amount of the disclosed compounds can range from about 0.001 mg/kg to about 1000 mg/kg, more preferably 0.01 mg/kg to about 500 mg/kg, more preferably 1 mg/kg to about 200 mg/kg. Effective doses will also vary, as recognized by those skilled in the art, depending on the diseases treated, route of administration, excipient usage, and the possibility of co-usage with other therapeutic treatments such as use of other agents.

The disclosed compounds can be co-administered with anti-cancer agents or chemotherapeutic agents such as alkylating agents, antimetabolites, natural products, hormones, metal coordination compounds, or other anticancer drugs. Examples of alkylating agents include nitrogen mustards (e.g., cyclophosphamide), ethylenimine and methylmelamines (e.g., hexamethlymelamine, thiotepa), alkyl sulfonates (e.g., busulfan), nitrosoureas (e.g., streptozocin), or triazenes (decarbazine, etc.). Examples of antimetabolites include folic acid analogs (e.g., methotrexate), pyrimidine analogs (e.g., fluorouracil), purine analogs (e.g., mercaptopurine). Examples of natural products include vinca alkaloids (e.g., vincristine), epipodophyllotoxins (e.g., etoposide), antibiotics (e.g., doxorubicin,), enzymes (e.g., L-asparaginase), or biological response modifiers (e.g., interferon alpha). Examples of hormones and antagonists include adrenocorticosteroids (e.g., prednisone), progestins (e.g., hydroxyprogesterone), estrogens (e.g., diethylstilbestrol), antiestrogen (e.g., tamoxifen), androgens (e.g., testosterone), antiandrogen (e.g., flutamide), and gonadotropin releasing hormone analog (e.g., leuprolide). Other agents that can be used in the methods and with the compositions of the invention for the treatment or prevention of cancer include platinum coordination complexes (e.g., cisplatin, carboblatin), anthracenedione (e.g., mitoxantrone), substituted urea (e.g., hydroxyurea), methyl hydrazine derivative (e.g., procarbazine), or adrenocortical suppressants (e.g., mitotane).

In various embodiments compounds can be coadministered with compounds that can inhibit angiogenesis or inhibit angiogenic tubule formation include, for example, matrix metalloproteinase inhibitors(dalteparin, suramin), endothelial cell inhibitors (e.g., thalidomide, squalamine, 2-methoxyestradiol), inhibitors of angiogenesis activation (e.g., avastatin, endostatin), celecoxib and the like.

### VII. METHODS OF PREPARATION

Processes for preparing compounds the disclosed compounds and intermediates that are useful in the preparation of such compounds, and processes for preparing such intermediates are also provided herein.

The compounds disclosed herein can be prepared according to the methods described in U.S. application Ser. No. 11/562,903, the entire teachings of which are incorporated herein by reference. The methods described in U.S. application Ser. No. 11/562,903 can be modified or augmented by synthetic chemistry functional group transformations known in the art and include, for example, those described in R. Larock, Comprehensive Organic Transformations, VCH Publishers (1989); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995). Comprehensive Organic Synthesis, Ed. B. M. Trost and I. Fleming (Pergamon Press, 1991), Comprehensive Organic Functional Group Transformations, Ed. A. R. Katritzky, O. Meth-Cohn, and C. W. Rees (Pergamon Press, 1996), Comprehensive Organic Functional Group Transformations II, Ed. A. R. Katritzky and R. J. K. Taylor (Editor) (Elsevier, 2^{nd} Edition, 2004), Comprehensive Heterocyclic Chemistry, Ed. A. R. Katritzky and C. W. Rees (Pergamon Press, 1984), and Comprehensive Heterocyclic Chemistry II, Ed. A. R. Katritzky, C. W. Rees, and E. F. V. Scriven (Pergamon Press, 1996). The entire teachings of these documents are are incorporated herein by reference.

Compounds of formula I may be prepared by the reaction compounds of formula III, wherein LG represents a suitable leaving group, by reaction with a compound of formula IV.

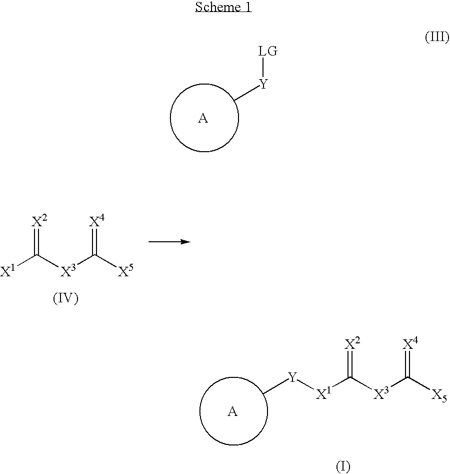

Suitable leaving groups LG in the compounds of formula III include halogen, particularly chlorine, bromine, and iodine, and sulfonate groups, particularly methanesulfonate, p-toluenesulfonate, and trifluoromethanesulfonate. The reactions are typically performed in a solvent at a suitable temperature. In some cases a base may be used as a catalyst. Suitable bases include alkali metal hydroxide or alkoxide salts such as sodium hydroxide or methoxide, and tertiary amines such as triethylamine or N,N-diisopropylethylamine. Suitable solvents include alcohols, such as methanol and ethanol, or dichloromethane. The reactions may be carried out at a temperature between 0° C. and the reflux temperature of the solvent, which is typically about 100° C. The reactions may be performed at a higher temperature by performing the reaction under pressure or in a sealed vessel. Microwave heating may be used. In a typical procedure, the components are reacted in by performing microwave heating, for example in ethanol at a temperature from about 80 to about 120° C.

Compounds of formula III are either commercially available, known in the art, or may be prepared by methods known to one skilled in the art. For example, —CH— groups alpha to an aromatic ring can be readily halogenated under free radical conditions. Alternatively, appropriate leaving groups could be introduced by conversion of the corresponding alcohol (by conversion of OH to halogen, or treatment with a sulfonyl chloride such as p-toluenesulfonyl chloride), which can be prepared by a variety of methods, for example, reduction of a aromatic carboxylic acid or an aromatic aldehyde or ketone.

Compounds of formula IV are either commercially available, known in the art, or may be prepared by methods known to one skilled in the art. For example, amidinothiourea (2-imino-4-thiobiuret) (CAS registry no. 2114-02-5) is commercially available from Sigma-Aldrich and other suppliers.

Compounds of formula II may be prepared by the reaction compounds of formula V, wherein LG represents a suitable leaving group, by reaction with a compound of formula

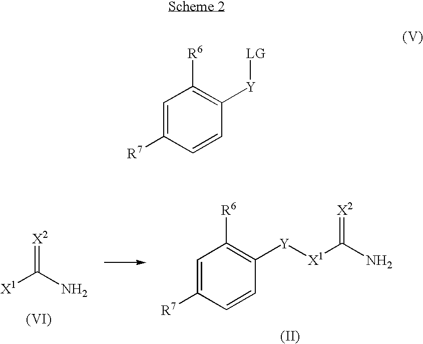

Suitable leaving groups LG in the compounds of formula IV include halogen, particularly chlorine, bromine, and iodine, and sulfonate groups, particularly methanesulfonate, p-toluenesulfonate, and trifluoromethanesulfonate. The reactions are typically performed in a solvent at a suitable temperature. In some cases a base may be used as a catalyst. Suitable bases include alkali metal hydroxide or alkoxide salts such as sodium hydroxide or methoxide, and tertiary amines such as triethylamine or N,N-diisopropylethylamine. Suitable solvents include alcohols, such as methanol and ethanol, or dichloromethane. The reactions may be carried out at a temperature between 0° C. and the reflux temperature of the solvent, which is typically about 100° C. The reactions may be performed at a higher temperature by performing the reaction under pressure or in a sealed vessel. Microwave heating may be used. In a typical procedure, the components are reacted in by performing microwave heating, for example in ethanol at a temperature from about 80 to about 120° C.

Compounds of formula V, such as benzyl halides, are either commercially available, known in the art, or may be prepared by methods known to one skilled in the art. For example, —CH— groups alpha to benzene ring can be readily halogenated under free radical conditions.

Alternatively, appropriate leaving groups could be introduced by conversion of the corresponding alcohol (by conversion of OH to halogen, or treatment with a sulfonyl chloride such as p-toluenesulfonyl chloride), which can be prepared by a variety of methods, for example, reduction of a benzoic acid or a benzaldehyde or phenyl ketone.

Compounds of formula VI are either commercially available, known in the art, or may be prepared by methods known to one skilled in the art. For example, thiourea (CAS registry no. 62-56-6) is commercially available from Sigma-Aldrich and other suppliers.

The above-described reactions, unless otherwise noted, are usually conducted at a pressure of about one to about three atmospheres, such as at ambient pressure (about one atmosphere).

In some embodiments, the compounds according to formula I or II may be used as isolated compounds. The expression “isolated compound” refers to a preparation of a compound of formula I or II, wherein the isolated compound has been separated from the reagents used, and/or byproducts formed, in the synthesis of the compound or compounds.

“Isolated” does not necessarily mean that the preparation is technically pure (homogeneous), but can mean that it is sufficiently pure to compound in a form in which it can be used therapeutically. The term “isolated compound” may refer to a preparation of a compound of formula I which contains the named compound or mixture of compounds according to formula I in an amount of at least 10 percent by weight of the total weight, at least 50 percent by weight of the total weight; at least 80 percent by weight of the total weight; at least 90 percent, at least 95 percent or at least 98 percent by weight of the total weight of the preparation.

The compounds of formula I and II and intermediates may be isolated from their reaction mixtures and purified by standard techniques such as filtration, liquid-liquid extraction, solid phase extraction, distillation, recrystallization or chromatography, including flash column chromatography, or HPLC. The preferred method for purification of the compounds according to formula I and II or salts thereof comprises crystallizing the compound or salt from a solvent to form, preferably, a crystalline form of the compounds or salts thereof. Following crystallization, the crystallization solvent is removed by a process other than evaporation, for example filtration or decanting, and the crystals are then preferably washed using pure solvent (or a mixture of pure solvents). Suitable solvents for crystallization include water, alcohols, particularly alcohols containing up to four carbon atoms such as methanol, ethanol, isopropanol, and butan-1-ol, butan-2-ol, and 2-methyl-2-propanol, ethers, for example diethyl ether, diisopropyl ether, t-butyl methyl ether, 1,2-dimethoxyethane, tetrahydrofuran and 1,4-dioxane, carboxylic acids, for example formic acid and acetic acid, and hydrocarbon solvents, for example pentane, hexane, toluene, and mixtures thereof, particularly aqueous mixtures such as aqueous ethanol. Pure solvents, preferably at least analytical grade, and more preferably pharmaceutical grade are preferably used. In a preferred embodiment of the processes, the products are so isolated. In the some embodiments of compounds according to formula I and II or salts thereof, and pharmaceutical compositions thereof, the compound according to formula I and II or salt thereof is in or prepared from a crystalline form, which may be prepared by crystallization according to such a process.

It will be appreciated by one skilled in the art that certain aromatic substituents in the compounds of formula I and II, intermediates used in the processes described above, or precursors thereto, may be introduced by employing aromatic substitution reactions to introduce or replace a substituent, or by using functional group transformations to modify an existing substituent, or a combination thereof. Such reactions may be effected either prior to or immediately following the processes mentioned above. The reagents and reaction conditions for such procedures are known in the art. Specific examples of procedures which may be employed include, but are not limited to, electrophilic functionalization of an aromatic ring, for example via nitration, halogenation, or acylation; transformation of a nitro group to an amino group, for example via reduction, such as by catalytic hydrogenation; acylation, alkylation, or sulfonylation of an amino or hydroxyl group; replacement of an amino group by another functional group via conversion to an intermediate diazonium salt followed by nucleophilic or free radical substitution of the diazonium salt; or replacement of a halogen by another group, for example via nucleophilic or organometallically-catalyzed substitution reactions.

In implementing preparations of the disclosed compounds functional groups which would be sensitive to the reaction conditions may be protected by protecting groups. A protecting group is a derivative of a chemical functional group which would otherwise be incompatible with the conditions required to perform a particular reaction which, after the reaction has been carried out, can be removed to re-generate the original functional group, which is thereby considered to have been “protected”. Any chemical functionality that is a structural component of any of the reagents used to synthesize compounds described herein may be optionally protected with a chemical protecting group if such a protecting group is useful in the synthesis of compounds described herein. The person skilled in the art knows when protecting groups are indicated, how to select such groups, and processes that can be used for selectively introducing and selectively removing them, because methods of selecting and using protecting groups have been extensively documented in the chemical literature. As used herein, “suitable protecting groups” and strategies for protecting and deprotecting functional groups using protecting groups useful in synthesizing the disclosed compounds are known in the art and include, for example, those described in T. W. Greene and P. G. M. Wuts, Protective Groups in Organic Synthesis, John Wiley and Sons (2nd Ed. 1991) or 4^{th} Ed. (2006), the entire teachings of which are incorporated herein by reference. For example, suitable hydroxyl protecting groups include, but are not limited to substituted methyl ethers (e.g., methoxymethyl, benzyloxymethyl) substituted ethyl ethers (e.g., ethoxymethyl, ethoxyethyl)benzyl ethers (benzyl, nitrobenzyl, halobenzyl) silyl ethers (e.g., trimethylsilyl), esters, and the like. Examples of suitable amine protecting groups include benzyloxycarbonyl, tert-butoxycarbonyl, tert-butyl, benzyl and fluorenylmethyloxy-carbonyl (Fmoc). Examples of suitable thiol protecting groups include benzyl, tert-butyl, acetyl, methoxymethyl and the like.

The reactions described herein may be conducted in any suitable solvent for the reagents and products in a particular reaction. Suitable solvents are those that facilitate the intended reaction but do not react with the reagents or the products of the reaction. Suitable solvents can include, for example: ethereal solvents such as diethyl ether or tetrahydrofuran; ketone solvents such as acetone or methyl ethyl ketone; halogenated solvents such as dichloromethane, chloroform, carbon tetrachloride, or trichloroethane; aromatic solvents such as benzene, toluene, xylene, or pyridine; polar aprotic organic solvents such as acetonitrile, dimethyl sulfoxide, dimethyl formamide, N-methylpyrrolidone, hexamethyl phosphoramide, nitromethane, nitrobenzene, or the like; polar protic solvents such as methanol, ethanol, propanol, butanol, ethylene glycol, tetraethylene glycol, or the like; nonpolar hydrocarbons such as pentane, hexane, cyclohexane, cyclopentane, heptane, octance, or the like; basic amine solvents such as pyridine, triethylamine, or the like; and other solvents known to the art.

Reactions or reagents which are water sensitive may be handled under anhydrous conditions. Reactions or reagents which are oxygen sensitive may be handled under an inert atmosphere, such as nitrogen, helium, neon, argon, and the like. Reactions or reagents which are light sensitive may be handled in the dark or with suitably filtered illumination.

Reactions or reagents which are temperature-sensitive, e.g., reagents that are sensitive to high temperature or reactions which are exothermic may be conducted under temperature controlled conditions. For example, reactions that are strongly exothermic may be conducted while being cooled to a reduced temperature.

Reactions that are not strongly exothermic may be conducted at higher temperatures to facilitate the intended reaction, for example, by heating to the reflux temperature of the reaction solvent. Reactions can also be conducted under microwave irradiation conditions. For example, in various embodiments of the method, the first and second reagents are reacted together under microwave irradiation.

Reactions may also be conducted at atmospheric pressure, reduced pressure compared to atmospheric, or elevated pressure compared to atmospheric pressure. For example, a reduction reaction may be conducted in the presence of an elevated pressure of hydrogen gas in combination with a hydrogenation catalyst.

Reactions may be conducted at stoichiometric ratios of reagents, or where one or more reagents are in excess.

### VIII. ASSAY METHODS

The disclosed compounds can be assayed for binding and biological activity by any means described herein or known to the art. For example, the disclosed compounds can be screened for binding activity in an ELISA assay (see Methods), the IC₅₀ values of the disclosed compounds can be determined by in vitro binding assays (see Methods), the binding selectivity of the disclosed compounds can be measured in competitive ELISA assays, and the ability of the disclosed compounds to disrupt Rb:Raf-1 in vitro or in vivo can be assayed.

Further, the disclosed compounds can be tested for their ability to kill or inhibit the growth of tumor cells or angiogenic tubules. Suitable assays include, for example, (a) tumor cell in anchorage/independent growth (soft agar assays); (b) tumor cell in anchorage-dependent growth (3-(4,5-Dimethylthiazol-2-yl)-2,5-diphenyltetrazolium bromide (MTT), trypan blue and DNA synthesis assays); (c) tumor cell survival (TUNEL, PARP cleavage, caspace activation and other apoptosis assays); (d) tumor cell invasion and metastasis; (e) endothelial cell migration, invasion and angiogenesis; (f) tumor cell proliferation inhibition assays; (g) anti-tumor activity assays in animal models; and other such assays known to the art.

Certain assays can be used to assess a subject for treatment with an inhibitor of Rb:Raf-1 binding interactions or to identify a subject for therapy. The level of Rb, Raf-1, or Rb bound to Raf-1 can be determined in the subject or in a sample from the subject, e.g., a subject with a cell proliferation disorder. Treatment with the disclosed compounds is indicated when the level of Rb, Raf-1, or Rb bound to Raf-1 is elevated compared to normal. “Elevated compared to normal” means that the levels are higher than in a reference sample of cells of the same type that are healthy. For example, the level of Rb, Raf-1, or Rb bound to Raf-1 in cells from a non-small cell lung cancer tumor can be compared to the level of Rb, Raf-1, or Rb bound to Raf-1 in normal, noncancerous cells. For example, Enzyme Linked ImmunoSorbent Assay (ELISA) can be used in combination with antibodies to Rb, Raf-1, or Rb bound to Raf-1 (see Methods, In vitro library screening assays). The assay can be embodied in a kit. For example, a kit includes a reagent or indicator, such as an antibody, that is specific for Rb, Raf-1, or Rb bound to Raf-1. The kit can also include instructions for determining the level of Rb, Raf-1, or Rb bound to Raf-1 in a sample using the reagent or indicator, such as an antibody, that is specific for Rb, Raf-1, or Rb bound to Raf-1.

### In Vitro/In Vivo

In various embodiments, methods relating to cells can be conducted on cells in vitro or in vivo, particularly wherein the cell is in vivo, i.e., the cell is located in a subject. A “subject” can be any animal with a proliferative disorder, for example, mammals, birds, reptiles, or fish. Preferably, the animal is a mammal. More preferably, the mammal is selected from the group consisting of dogs, cats, sheep, goats, cattle, horses, pigs, mice, non-human primates, and humans. Most preferably, the mammal is a human.

### IX. THERAPEUTIC METHODS AND USES OF THE COMPOUNDS

Described herein are methods of using the disclosed compounds. The disclosed compounds are useful in inhibiting the Rb-Raf-1 binding. The disclosed compounds are biologically active and therapeutically useful.

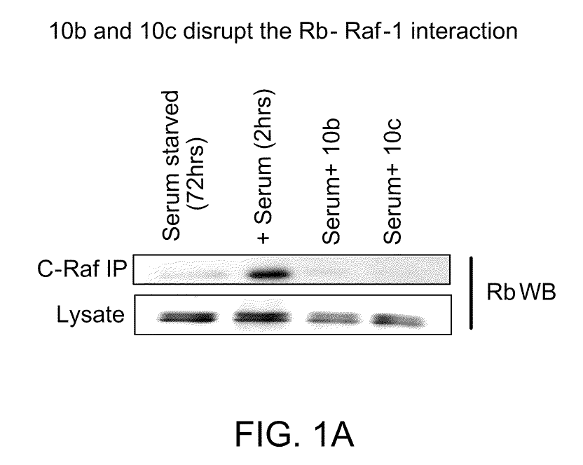

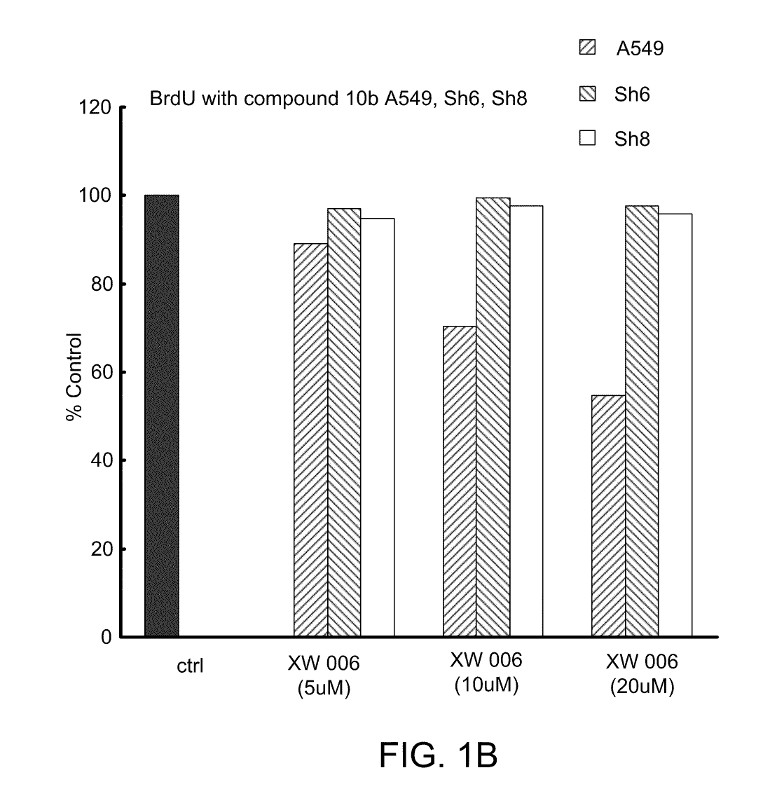

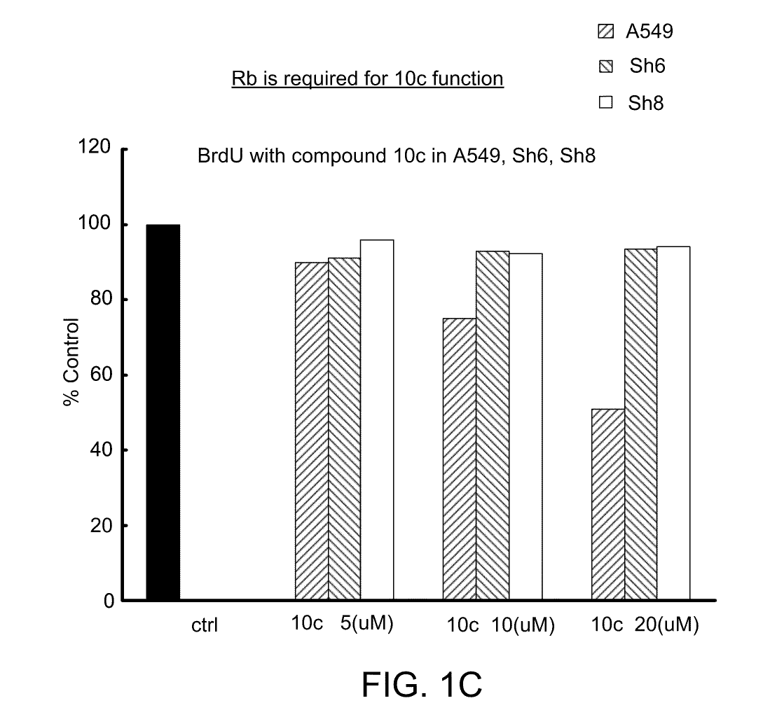

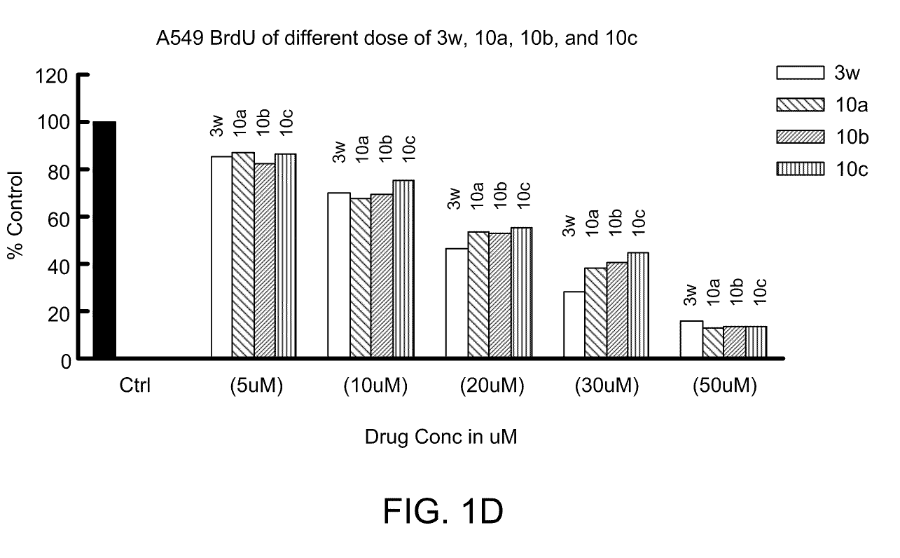

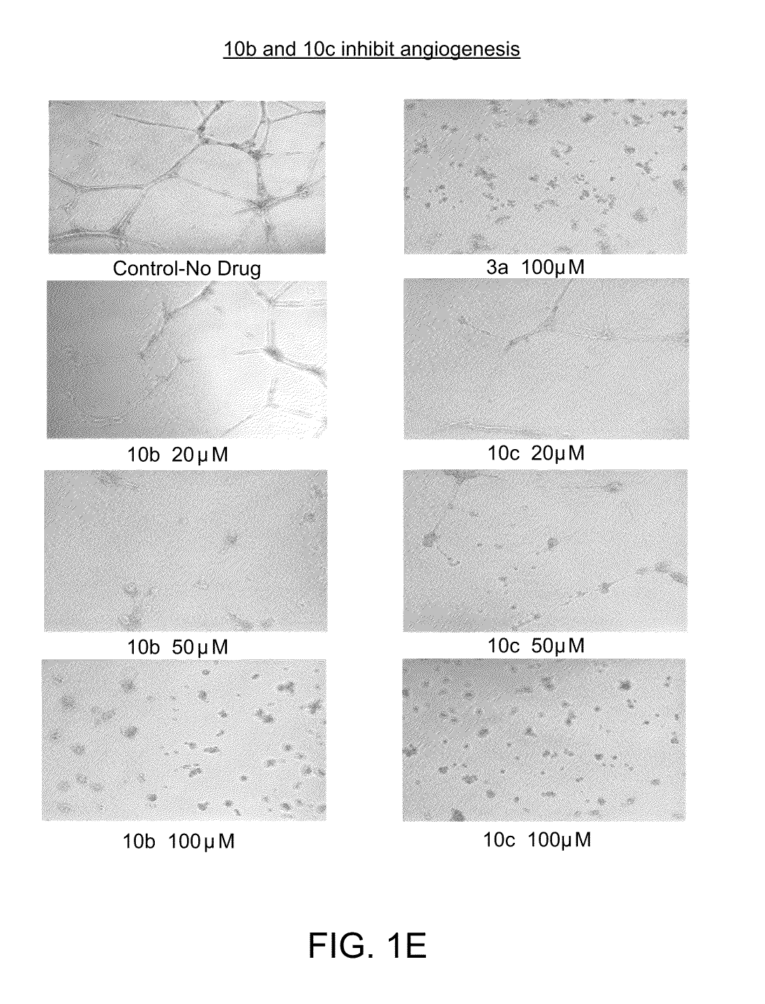

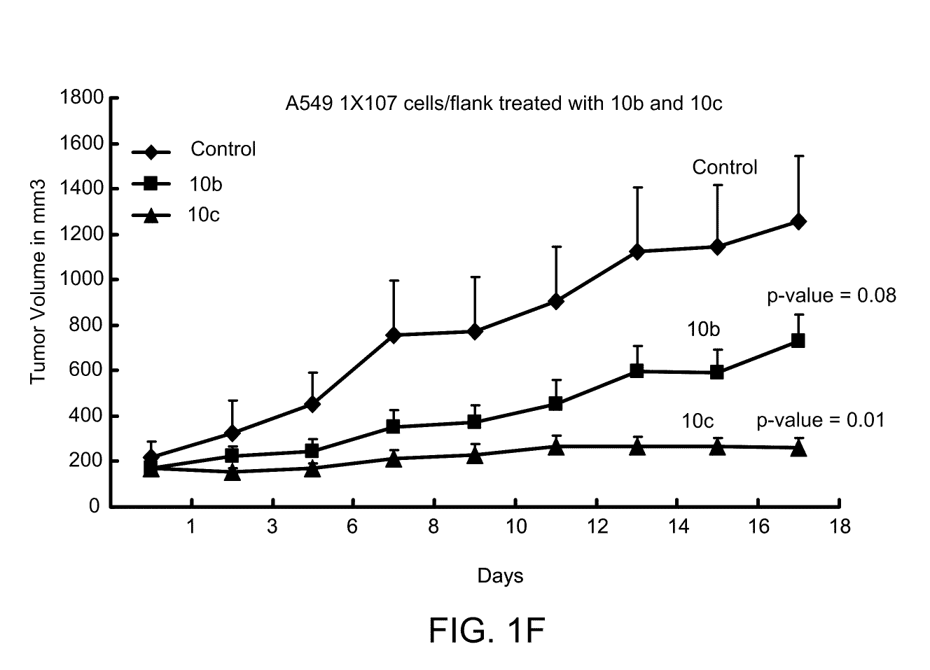

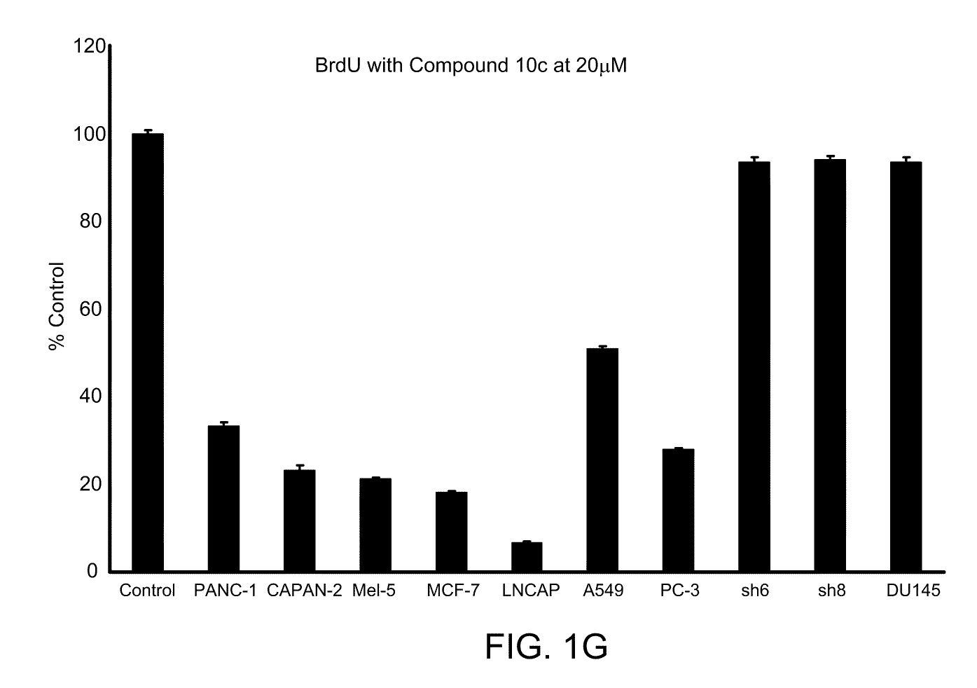

Evidence for the therapeutic utility of inhibitors of Rb-Raf-1 binding was presented in WO2007/062222, which is incorporated herein by reference in its entirety, particularly the results described in Examples 5 to 20 and in FIGS. 1-4A of that application, which are also incorporated herein by reference. In that application, compounds which modulated Rb:Raf-1 modulators selectively over Rb:E2F1 were described. The molecules were able disrupt Rb:Raf-1 in vitro as well as in intact cells. Compound 3a was found to inhibit the proliferation of Rb-expressing osteosarcoma cells (U2-OS), human epithelial lung carcinoma cells (A549), non-small cell lung cancer cells (H1650), pancreatic cancer cells (Aspc1, PANC1, and CAPAN2), glioblastoma cells (U87MG and U251MG), metastatic breast cancer cells (MDA-MB-231), melanoma cells (A375), prostate cancer cells (LNCaP and PC3). The compounds also inhibited the adherence-independent growth of various types of cancer cells A549 (human epithelial lung carcinoma), H1650 (NSCLC), SK-MEL-5, SK-MEL-28 (melanoma), and PANC1 (pancreatic) cells in soft agar. The compounds were believed to exert their anti-cancer effects through disruption of the Rb:Raf-1 interation. The inhibitors of Rb:Raf-1 binding also disrupted angiogenesis. Inhibitors of Rb-Raf-1 binding were also shown to inhibit proliferation of a human tumor cell line (A549) in vivo in a nude mouse xenografts model.

The Ras/Raf/Mek/MAPK cascade is a proliferative pathway induced by a wide array of growth factors and is activated in many human tumors. It has been shown that signaling pathways through the MAP kinase cascade do not proceed in a linear fashion, but rather that they have been found to have substrates outside the cascade as well. Without wishing to be bound by theory, in this context, the Rb protein appears to be an important cellular target of the Raf-1 kinase outside the MAP kinase cascade. The binding of Raf-1 to Rb was found to occur only in proliferating cells and contributed to cell cycle progression. Further, it was found that the level of Rb:Raf-1 interaction was elevated in NSCLC tissue, suggesting that it may have contributed to the oncogenic process. These observations support the hypothesis that targeting the Rb:Raf-1 interaction with the disclosed compounds is a viable method to develop anticancer drugs.

The cell-permeable, orally available, and target specific small molecule compound 3a, can maintain the tumor suppressor functions of Rb. The in vitro results indicate that compound 3a selectively inhibits the Rb:Raf-1 interaction without targeting the binding partners of Rb and Raf-1, such as E2F1, prohibition, HDAC1 and MEK½. Further, compound 3a functions by inhibiting the interaction of Raf-1 and Rb without inhibiting Raf-1 kinase activity or the kinase activity associated with cyclins D or E. Also, compound 3a inhibited cell cycle and decreased the levels of cyclin D while cdk activity was unaffected. Compound 3a demonstrated Rb dependence to inhibit cell cycle progression and tumor growth in cell lines. These results further confirm the specificity of 3a for targeting Rb:Raf-1. Mice harboring A549 tumors responded to treatment with 3a administered by i.p. or oral gavage. Tumor tissue displayed a decrease in proliferation, Rb phosphorylation, and angiogenesis and an increase in apoptosis. Importantly, A-549 tumors where Rb was knockdown are resistant to 3a, further suggesting that 3a inhibits tumor growth by targeting the Rb:Raf-1 interaction.

These results show that the mechanism of 3a mediated growth arrest is likely by targeting the Rb:Raf-1 interaction. Aberrant signaling mechanisms surrounding the Ras/MAPK and Rb/E2F1 pathways are commonly present in cancers. The disclosed compounds, such as compound 3a, could inhibit S-phase entry in potentially 35%-90% of all of the cell lines. Based on the substantial in vitro and in vivo results disclosed herein, it is believed that the disclosed compounds, in particular compound 3a, are excellent candidates for the treatment of cancer patients whose tumors harbor genetic aberrations that lead to inactivation of Rb by Raf-1.

The compounds, pharmaceutical compositions, and methods of treatment described in this application are believed to be effective for inhibiting cellular proliferation, particularly of cells which proliferate due to a mutation or other defect in the Rb:Raf-1 regulatory pathway. The disclosed compounds, pharmaceutical compositions, and methods of treatment are therefore believed to be effective for treating cancer and other proliferative disorders which can be inhibited by disrupting Rb:Raf-1 binding interactions in the proliferating cells.

The disclosed compounds can participate in a protein-ligand complex. A protein:ligand complex includes a compound and at least one protein selected from the group consisting of retinoblastoma tumor suppressor protein and serine-threonine kinase Raf-1.

The complex can include a disclosed compound, retinoblastoma tumor suppressor protein, and serine-threonine kinase Raf-1.

Various methods of treatment of cells and subjects are provided. For example, a method of inhibiting proliferation of a cell includes contacting the cell with an effective amount of the disclosed compounds or compositions. Typically, regulation of proliferation in the cell is mediated by at least one protein selected from the group consisting of retinoblastoma tumor suppressor protein and serine-threonine kinase Raf-1. For example, in various embodiments, the cells have an elevated level of Rb, Raf-1, or Rb bound to Raf-1. In some embodiment, the method includes assaying the level of Rb, Raf-1, or Rb bound to Raf-1 in the cell.

A method of modulating the Rb:Raf-1 interaction in a proliferating cell is provided. The method includes contacting the cell with an effective amount of the disclosed compounds or compositions.

A method of modulating the Rb:Raf-1 interaction in a proliferating cell is provided. The method includes contacting the cell with a modulator of the Rb:Raf-1 interaction that is suitable for oral administration. In some embodiments, the modulator of the Rb:Raf-1 interaction is orally administered.

A method of treating or ameliorating a cell proliferation disorder is provided. The method includes contacting the proliferating cells with an effective amount of the disclosed compounds or compositions. Typically, regulation of cell proliferation in the disorder can be mediated by at least one protein selected from the group consisting of retinoblastoma tumor suppressor protein and serine-threonine kinase Raf-1. The regulation of proliferation in the cells may be mediated by the interaction between retinoblastoma tumor suppressor protein and serine-threonine kinase Raf-1. The cell proliferation disorder may be cancer or a non-cancerous cell proliferation disorder. The cell proliferation disorder may include angiogenesis or the cell proliferation disorder may be mediated by angiogenesis.

A method of treating or ameliorating a cell proliferation disorder may also include administering the compound, or a pharmaceutically acceptable salt thereof, to a patient in need of such treatment.

In various embodiments, the cell proliferation disorder is or the proliferating cells are derived from a cancerous or a non-cancerous cell proliferation disorder. Exemplary cancerous and non-cancerous cell proliferation disorders include fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, non-small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, acute lymphocytic leukemia, lymphocytic leukemia, large granular lymphocytic leukemia, acute myelocytic leukemia, chronic leukemia, polycythemia vera, Hodgkin's lymphoma, non-Hodgkin's lymphoma, multiple myeloma, Waldenstrobm's macroglobulinemia, heavy chain disease, lymphoblastic leukemia, T-cell leukemia, T-lymphocytic leukemia, T-lymphoblastic leukemia, B cell leukemia, B-lymphocytic leukemia, mixed cell leukemias, myeloid leukemias, myelocytic leukemia, myelogenous leukemia, neutrophilic leukemia, eosinophilic leukemia, monocytic leukemia, myelomonocytic leukemia, Naegeli-type myeloid leukemia, nonlymphocytic leukemia, osteosarcoma, promyelocytic leukemia, non-small cell lung cancer, epithelial lung carcinoma, pancreatic carcinoma, pancreatic ductal adenocarcinoma, glioblastoma, metastatic breast cancer, melanoma, and prostate cancer. In certain embodiments, the cell proliferation disorder is osteosarcoma, promyelocytic leukemia, non-small cell lung cancer, epithelial lung carcinoma, pancreatic carcinoma, pancreatic ductal adenocarcinoma, glioblastoma, metastatic breast cancer, melanoma, or prostate cancer.

A method of inhibiting angiogenic tubule formation in a subject in need thereof includes administering to the subject an effective amount of the disclosed compounds or compositions.

In some embodiments, the preceding methods of treating subjects or cells can also include coadministration of an anticancer drug or a compound that modulates angiogenic tubule formation, particularly coadministration of a compound that inhibits angiogenic tubule formation. Exemplary anticancer drugs and compounds that can modulate angiogenic tubule

Examples of suitable chemotherapeutic agents include any of abarelix, aldesleukin, alemtuzumab, alitretinoin, allopurinol, altretamine, anastrozole, arsenic trioxide, asparaginase, azacitidine, bevacizumab, bexarotene, bleomycin, bortezombi, bortezomib, busulfan intravenous, busulfan oral, calusterone, capecitabine, carboplatin, carmustine, cetuximab, chlorambucil, cisplatin, cladribine, clofarabine, cyclophosphamide, cytarabine, dacarbazine, dactinomycin, dalteparin sodium, dasatinib, daunorubicin, decitabine, denileukin, denileukin diftitox, dexrazoxane, docetaxel, doxorubicin, dromostanolone propionate, eculizumab, epirubicin, erlotinib, estramustine, etoposide phosphate, etoposide, exemestane, fentanyl citrate, filgrastim, floxuridine, fludarabine, fluorouracil, fulvestrant, gefitinib, gemcitabine, gemtuzamab ozogamicin, goserelin acetate, histrelin acetate, ibritumomab tiuxetan, idarubicin, ifosfamide, imatinib mesylate, interferon alfa 2a, irinotecan, lapatinib ditosylate, lenalidomide, letrozole, leucovorin, leuprolide acetate, levamisole, lomustine, mechlorethamine, megestrol acetate, melphalan, mercaptopurine, methotrexate, methoxsalen, mitomycin C, mitotane, mitoxantrone, nandrolone phenpropionate, nelarabine, nofetumomab, oxaliplatin, paclitaxel, pamidronate, panitumumab, pegaspargase, pegfilgrastim, pemetrexed disodium, pentostatin, pipobroman, plicamycin, procarbazine, quinacrine, rasburicase, rituximab, sorafenib, streptozocin, sunitinib, sunitinib maleate, tamoxifen, temozolomide, teniposide, testolactone, thalidomide, thioguanine, thiotepa, topotecan, toremifene, tositumomab, trastuzumab, tretinoin, uracil mustard, valrubicin, vinblastine, vincristine, vinorelbine, vorinostat, and zoledronate.

A method of assessing a subject for treatment with an inhibitor of Rb:Raf-1 binding interactions includes determining, in the subject or in a sample from the subject, a level of Rb, Raf-1, or Rb bound to Raf-1, wherein treatment with an inhibitor of Rb:Raf-1 binding interactions is indicated when the level of Rb, Raf-1, or Rb bound to Raf-1 is elevated compared to normal.

A method of identifying a subject for therapy includes the steps of providing a sample from the subject, determining a level of Rb, Raf-1, or Rb bound to Raf-1 in the sample; and identifying the subject for therapy with an inhibitor of Rb:Raf-1 binding interactions when the level of Rb, Raf-1, or Rb bound to Raf-1 is elevated compared to normal.

A kit includes an antibody specific for Rb, Raf-1, or Rb bound to Raf-1; and instructions for determining the level of Rb, Raf-1, or Rb bound to Raf-1 in a sample using the antibody specific for Rb, Raf-1, or Rb bound to Raf-1.

In various embodiments, methods relating to cells can be conducted on cells in vitro or in vivo, particularly wherein the cell is in vivo in a subject. The subject can be, for example, a bird, a fish, or a mammal, e.g., a human.

The compounds according to the invention may be administered to individuals (mammals, including animals and humans) afflicted with a cell proliferation disorder such as cancer, malignant and benign tumors, blood vessel proliferative disorders, autoimmune disorders, and fibrotic disorders.

The compounds are believed effective against a broad range of tumor types, including but not limited to the following: ovarian cancer; cervical cancer; breast cancer; prostate cancer; testicular cancer, lung cancer, renal cancer; colorectal cancer; skin cancer; brain cancer; leukemia, including acute myeloid leukemia, chronic myeloid leukemia, acute lymphoid leukemia, and chronic lymphoid leukemia. Examples of cancers include fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, non-small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, acute lymphocytic leukemia, lymphocytic leukemia, large granular lymphocytic leukemia, acute myelocytic leukemia, chronic leukemia, polycythemia vera, Hodgkin's lymphoma, non-Hodgkin's lymphoma, multiple myeloma, Waldenstrobm's macroglobulinemia, heavy chain disease, lymphoblastic leukemia, T-cell leukemia, T-lymphocytic leukemia, T-lymphoblastic leukemia, B cell leukemia, B-lymphocytic leukemia, mixed cell leukemias, myeloid leukemias, myelocytic leukemia, myelogenous leukemia, neutrophilic leukemia, eosinophilic leukemia, monocytic leukemia, myelomonocytic leukemia, Naegeli-type myeloid leukemia, nonlymphocytic leukemia, osteosarcoma, promyelocytic leukemia, non-small cell lung cancer, epithelial lung carcinoma, pancreatic carcinoma, pancreatic ductal adenocarcinoma, glioblastoma, metastatic breast cancer, melanoma, or prostate cancer.

Cancers may be solid tumors that may or may not be metastatic. Cancers may also occur, as in leukemia, as a diffuse tissue. Thus, the term “tumor cell”, as provided herein, includes a cell afflicted by any one of the above identified disorders.

The compounds are also believed useful in the treatment of non-cancer cell proliferation disorders, that is, cell proliferation disorders which are characterized by benign indications. Such disorders may also be known as “cytoproliferative” or “hyperproliferative” in that cells are made by the body at an atypically elevated rate. In various embodiments, the non-cancerous cell proliferation disorder includes cells that have a mutation or defect in the Rb:Raf-1 pathway. Non-cancer cell proliferation disorders believed treatable by compounds according to the invention include, for example, smooth muscle cell proliferation, systemic sclerosis, cirrhosis of the liver, adult respiratory distress syndrome, idiopathic cardiomyopathy, lupus erythematosus, retinopathy, cardiac hyperplasia, benign prostatic hyperplasia, ovarian cysts, pulmonary fibrosis, endometriosis, fibromatosis, harmatomas, lymphangiomatosis, sarcoidosis, desmoid tumors, intimal smooth muscle cell hyperplasia, restenosis, vascular occlusion, hyperplasia in the bile duct, hyperplasia in the bronchial airways, hyperplasia in the kidneys of patients with renal interstitial fibrosis, psoriasis, Reiter's syndrome, pityriasis rubra pilaris, a hyperproliferative disorder of keratinization, or scleroderma.

In various embodiments, the cancer includes cells that have a mutation or defect in the Rb:Raf-1 pathway. In certain embodiments, the cancer is osteosarcoma, promyelocytic leukemia, non-small cell lung cancer, epithelial lung carcinoma, pancreatic carcinoma, pancreatic ductal, adenocarcinoma, glioblastoma, metastatic breast cancer, melanoma, or prostate cancer.

The methods described above can be applied performed any of the compounds or embodiments thereof described in the Summary or Section II above, or their salts described in Section IV above. In particular, the methods can be carried out with any of the compounds whose structures are given below, particularly the 2,4-dichlorophenyl amindinoisothiourea whose structure is provided, or with salts of such compounds as described in Section IV above.

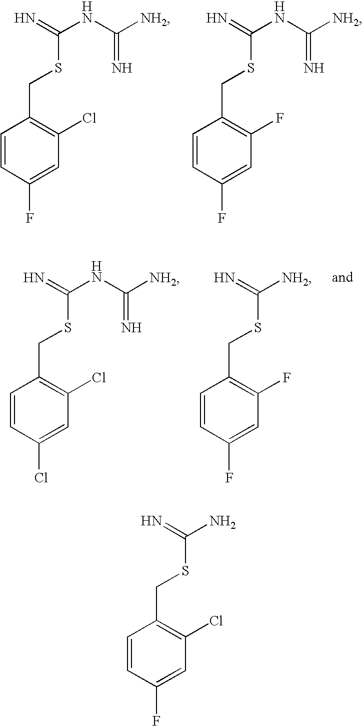

Based on the utilities described herein, the compounds disclosed or claimed herein are provided for use in medicine. The compounds are also provided for use in the therapeutic methods described or claimed herein, and for manufacturing a medicament for carrying out the therapeutic methods described or claimed herein.

### X. EXAMPLES

### Methods

Chemistry. All reagents were purchased from commercial suppliers and used without further purification. ¹H NMR spectra were recorded using a Mercury 400 NMR spectrometer (Varian, Palo Alto, Calif.). ¹³C NMR spectra were recorded at 100 MHz, in some cases using

Distortionless Enhancement by Polarization Transfer. Solvents employed were CDCl₃ or d₆-DMSO (dimethyl sulfoxide). All coupling constants are measured in Hertz (Hz) and the chemical shifts (δ_{H} and δ_{C}) are quoted in parts per million (ppm) relative to the internal standard, e.g., CDCl₃, d₆-DMSO, or TMS (tetramethyl silane). Atmospheric pressure ionization (API) and electrospray (ES) mass spectra and accurate mass determinations were recorded using a time of flight (TOF) mass spectrometer (Agilent 6210 LC/MS (ESI-TOF), Agilent/Hewlett Packard, Santa Clara, Calif.). Microwave reactions were performed in CEM 908005 model and Biotage initiator 8 machines. High Performance Liquid Chromatography (HPLC) analysis was performed using a HPLC system equipped with a PU-2089 Plus quaternary gradient pump and a UV-2075 Plus UV-VIS detector (JASCO, Easton, M D), e.g., using an Alltech Kromasil C-18 column (150×4.6 mm, 5 μm). Infra red spectra were recorded using a FTIR-4100 spectrometer (JASCO). Melting points were determined using either a MEL-TEMP Electrothermal melting point apparatus or a Barnstead international melting point apparatus and are uncorrected. Column chromatography was conducted using silica gel 63-200 mesh (Merck & Co., Whitehouse Station, N.J.). Silica thin layer chromatography (TLC) was conducted on pre-coated aluminum sheets (60 F₂₅₄, Merck & Co. or Fisher), with observation under UV when necessary. Anhydrous solvents (acetonitrile, dimethyl formamide, ethanol, isopropanol, methanol and tetrahydrofuran) were used as purchased from Aldrich. HPLC grade solvents (methanol, acetonitrile and water) were purchased from Burdick and Jackson for HPLC and mass analysis.

Cell culture and transfection. The human promyelocytic leukemia cell line U937 was cultured in RPMI (Mediatech, Hernden, Va.) containing 10% fetal bovine serum (FBS; Mediatech). U2-OS, Saos-2, MCF7, PANC1 and MDA-MB-231 cell lines were cultured in Dulbecco modified Eagle Medium (DMEM; Mediatech) containing 10% FBS. A549 cells and A549 shRNA Rb cell lines were maintained in Ham F-12K supplemented with 10% FBS. ShRNA cells lines were maintained in media containing 0.5 μg/mL puromycin. H1650, PC-9 and Aspc1 cell line were cultured in RPMI (Gibco/Invitrogen, Carlsbad, Calif.) containing 10% FBS. PANC1 and CAPAN2 pancreatic cell lines and the A375 Melanoma cell line was grown in DMEM supplemented with 10% FBS. Human aortic endothelial cells (HAECs, Clonetics, San Diego, Calif.) were cultured in endothelial growth medium, supplemented with 5% FBS, according to the manufacturer's instructions. U251MG and U87MG glioma cell lines were maintained in DMEM supplemented with non-essential amino acids, 50 mM β-mercaptoethanol, and 10% FBS. ShRNA cell lines were made by stably transfecting A549 cells with two different shRNA constructs that specifically target Rb obtained from a library. The adenovirus (Ad) constructs Ad-green fluorescent protein (GFP) and Ad-E2F1 were obtained from W. D. Cress. Ad-cyclin D was provided by I. Cozar-Castellano.
In vitro library screening assays. Enzyme Linked ImmunoSorbent Assay (ELISA) 96-well plates were coated with 1 μg/mL of a glutathione S-transferase (GST) Raf-1 (1-149aa) overnight at 4° C. Subsequently the plates were blocked and GST Rb at 20 μg/mL was rotated at room temperature (RT) for 30 minutes in the presence or absence of the compounds at 20 micromolar (μM). GST-Rb +/− compounds were then added to the plate and incubated for 90 minutes (min) at 37° C. The amount of Rb bound to Raf-1 was detected by Rb polyclonal antibody (Santa Cruz Biotechnology, Santa Cruz, Calif.) 1:1000 incubated for 60 min at 37° C. Donkey-anti-rabbit-IgG-HRP (1:10,000) was added to the plate and incubated at 37° C. for 60 minutes. The color was developed with orthophenylenediamine (Sigma, St. Louis, Mo.) and the reaction was terminated with 3 molar (M) H₂SO₄. Absorbance was read at 490 nanometers (nm). To determine disruption of Rb to E2F1, Phb, or HDAC1 the above protocol was used with the exception of coating GST Rb on the ELISA plate and adding the drugs in the presence or absence of GST E2F1, Phb, or HDAC1. E2F1 monoclonal antibody (1:2000) was used to detect the amount of Rb bound to E2F1. Prohibition monoclonal antibody was used at 1:1000 to detect the amount of Rb bound to Prohibition. For disruption of MEK-Raf-1 binding ELISAs, Raf-1 1 microgram/milliliter (μg/mL) was coated on the plate and GST-MEK (20 μg/mL) was incubated +/− the compounds for 30 minutes at room temperature. Mek1 polyclonal antibody was used at 1:1000 to detect the binding of Raf-1 to Mek1. The IC₅₀ concentrations for the Rb:Raf-1 inhibitors were determined by plotting with Origin 7.5 software (Origin, Northampton, Mass.).
In vitro binding assays. Glutathione S-transferase (GST) fusion of Rb, Raf-1, E2F1, and MEK1 have been previously described (Dasgupta P, Sun J, Wang S, et al. Mol Cell Biol 2004; 24(21):9527-9541). First, 200 micrograms (pig) of U937 asynchronous lysates were pre-incubated with 10 μM of the indicated drugs or 1 μM of the Raf-1 peptide for 30 minutes at 4° C. Next, 200 μg of the U937 lysates were incubated with glutathione beads carrying an equal amount of the GST fusion proteins in 200 μl of protein binding buffer (20 mM Tris [pH 7.5], 50 mM KCL, 0.5 mM EDTA, 1 mM dithiothreitol, 0.5% NP-40, 3 mg of bovine serum albumin/mL) at 4° C. for 2 h. (Wang S, Ghosh R, Chellappan S. Mol Cell Biol 1998; 18(12):7487-7498).
Matrigel Assays. Matrigel (Collaborative Biomedical Products) was used to promote the differentiation of HAECs into capillary tube-like structures (Dasgupta P, Sun J, Wang S, et al. Mol Cell Biol 2004; 24(21):9527-9541). A total of 100 μl of thawed Matrigel was added to 96-well tissue culture plates, followed by incubation at 37° C. for 60 minutes to allow polymerization. Subsequently, 1×10⁴ HAECs were seeded on the gels in EGM medium supplemented with 5% FBS in the presence or absence of 20 μM concentrations of the indicated compounds, followed by incubation for 24 hours at 37° C. Capillary tube formation assessed by using a Leica DMIL phase contrast microscope.
Lysate preparation, immunoprecipitation, and Western blotting. Lysates from cells treated with different agents were prepared by NP-40 lysis as described earlier (Wang 1998). Tumor lysates were prepared with T-Per tissue lysis buffer (Pierce) and a Fischer PowerGen 125 dounce homogenizer. Physical interaction between proteins in vivo was analyzed by immunoprecipitation-Western blot analyses with 200 μg of lysate with 1 μg of the indicated antibody as previously described (Wang 1998). Polyclonal E2F1 and Cyclin D were obtained from Santa Cruz Biotechnology. Monoclonal Rb and Raf-1 were supplied by BD Transduction laboratories (San Jose, Calif.). Polyclonal antibodies to phospho-Rb (807,811) phospho-MEK½, MEK½, phospho-Erk½ and ERK½ were supplied by Cell Signaling (Danvers, Mass.).
Chromatin Immunoprecipitation (ChIP) assay. A549 cells were rendered quiescent by serum starvation and re-stimulated with serum for 2 h or 16 h in the presence or absence of RRD 251 at 20 μM. Cells were cross-linked with 1% formaldehyde for 10 minutes at room temperature. Subsequently, the cells were harvested and lysates were prepared. Immunoprecipitations were analyzed for the presence of E2F1, Rb, Raf-1, Brg1, HP1, and HDAC1 by PCR as previously described (Dasgupta 2004). Rabbit anti-mouse secondary antibody was used as the control for all reactions. The sequences of the PCR primers used in the PCRs were as follows: Cdc6 promoter (forward primer), 5′-GGCCTCACAG CGACTCTAAGA-3′; and Cdc6 promoter (reverse primer), 5′-CTCGGACTCACCACAAGC-3′. TS promoter (forward primer), and 5′-GAC GGA GGC AGG CCA AGT G-3′ TS promoter (reverse primer). The cdc25A and c-fos primers are described in (Dasgupta, 2004).
In vitro kinase assay. The kinase reaction for Raf-1 was carried out with 100 nanograms (ng) of Raf-1 (Upstate Signaling, Charlottesville, Va.), 0.5 μg of full-length Rb protein (QED Bioscience, San Diego, Calif.) as the substrate, 10 μM ATP, 10 μCi of [γ-³²P] ATP in the kinase assay buffer in the presence or absence of the drugs at 30° C. for 30 minutes. Cyclin D and E kinase assays are described in (Dasgupta 2004).
Proliferation assays. Bromodeoxyuridine (BrdU) labeling kits were obtained from Roche Biochemicals (Indianapolis, Ind.). Cells were plated in poly-D-lysine coated chamber slides at a density of 10,000 cells per well and rendered quiescent by serum starvation for 24 hours. Cells were then re-stimulated with serum in the presence or absence of the indicated drugs for 18 h. S-phase cells were visualized by microscopy and quantitated by counting 3 fields of 100 in quadruplicate.
Soft Agar assay. Soft agar assays were done in triplicate in 12-well plates (Corning, Corning N.Y.). First, the bottom layer of agar (0.6%) was allowed to solidify at room temperature. Next the top layer of agar was (0.3%) was mixed with 5,000 cells per well and the indicated drug. The drugs were added twice weekly in complete media to the agar wells. Colonies were quantified by staining with MTT (3-(4,5-Dimethylthiazol-2-yl)-2,5-diphenyltetrazolium bromide) 1 mg/mL for 1 hour at 37° C.
Animal Studies. Nude mice (Charles River, Wilmington, Mass., USA) were maintained in accordance with Institutional Animal Care and Use Committee (IACUC) procedures and guidelines. A549 cells were harvested and resuspended in PBS, and then injected s.c. into the right and left flanks (10×10⁶ cells per flank) of 8-week old female nude mice as reported previously (Sun 99). When tumors reached about 100-200 mm³, animals were dosed intraperitoneally i.p. or orally by gavage with 0.1 mL solution once daily. Control animals received a vehicle, whereas treated animals were given compound at the indicated doses. The tumor volumes were determined by measuring the length (l) and the width (w) and calculating the volume (V=lw²/2) as described previously (Sun 99). Statistical significance between control and treated animals were evaluated using Student's t-test.
Immunohistochemistry staining. Upon termination of xenograft anti-tumor experiments, tumors were removed and fixed in 10% neutral-buffered formalin before processing into paraffin blocks. Tissue sections (5 micrometers (μm) thick) were cut from the blocks and stained with Ki-67, CD31, TUNEL, and phospho-Rb antibodies. Paraffin sections were rehydrated to PBS and processed using the following protocols. Sections were rinsed in dH₂O, and then subjected to microwave ‘antigen retrieval’ for 20 minutes on 70% power, with a 1 minute cooling period after every 5 minutes, in 0.01 M sodium citrate, pH 6.0 (Janssen P J, Brinkmann A O, Boersma W J, Van der Kwast T H. J Histochem Cytochem 1994; 42(8):1169-75; Shi S R, Key M E, Kalra K L. J Histochem Cytochem 1991; 39(6):741-748). Sections were cooled for 20 minutes, rinsed 3 times in dH₂O, twice in PBS and incubated in 5% normal goat serum for 30 minutes. Sections were incubated in primary antibody for 1 hour in 5% normal goat serum, rinsed 3 times in PBS. For color development the slides were treated with ABC kit (Vector Labs, Burlingame, Calif.) rinsed in dH₂O, and developed using DAB as chromogen. After a final rinse in dH₂O, sections were lightly counterstained in hematoxylin, dehydrated, cleared and coverslipped. Tissue sections were stained with hematoxylin and eosin (H&E) using standard histological techniques. Tissue sections were also subjected to immunostaining for CD31 (BD Biosciences, San Diego, Calif., USA) using the avidin-biotin peroxidase complex technique. Mouse monoclonal antibody was used at 1:50 dilution following microwave antigen retrieval (four cycles of 5 min each on high in 0.1 M citrate buffer). Apoptotic cells were detected using DeadEnd Colorimetric TUNEL system (Promega, Madison, Wis.).

### General Synthetic Procedures for Modulators of Rb:Raf 1 Interactions

Reference compounds 1 and 2 were discovered by screening a library of compounds using a glutathione S-transferase-retinoblastoma/glutathione S-transferase-Raf-1 kinase Enzyme-Linked ImmunoSorbent Assay screen (GST-Rb/GST-Raf-1 ELISA). Two structurally related compounds (1) and (2) were discovered that strongly inhibited the Rb:Raf-1 interaction at a concentration of 20 μM (100% for 1 and 95% for 2):

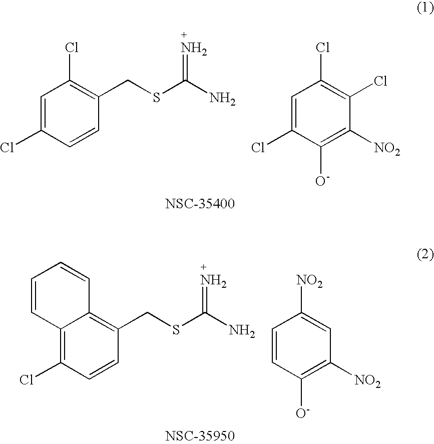

Benzylisothiourea derivatives 3, lacking substitution at the a benzylic position, are prepared in good yields by reaction of thiourea with the appropriate benzyl halide (Scheme 3, Table 1). (Yong 1997) When not commercially available the desired benzyl halides are obtained from the corresponding benzyl alcohols (prepared when necessary by NaBH₄ reduction of the corresponding aldehyde) followed by reaction with thionyl chloride to generate the corresponding benzyl chloride. The corresponding benzylisothiourea derivatives 3 are usually obtained in good to quantitative yields.

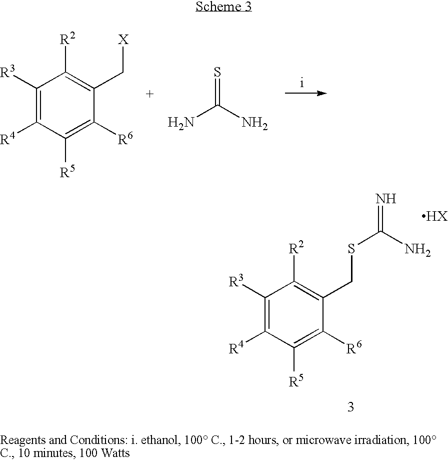

Amidinoisothiourea compounds 10a-j and 11a-b are synthesized according to Scheme 4.

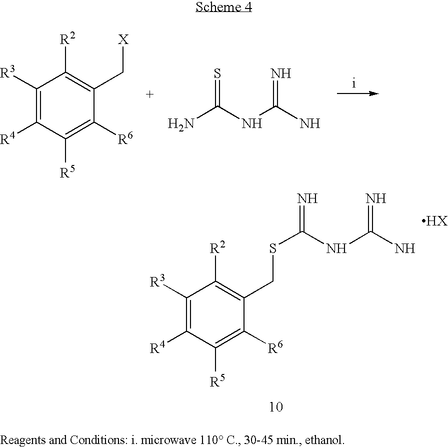

Benzylisothiouronium derivatives 4 bearing an alkyl group at the benzylic position may be prepared by the reaction of thiourea with the appropriate α-substituted benzyl halides. The α-substituted benzyl halides may be prepared by addition of an alkylmagnesium bromide to the appropriate benzaldehyde, followed by treatment of the intermediate alcohol with thionyl chloride. Substituted amidinoisothiourea compounds may be prepared by analogous methods.

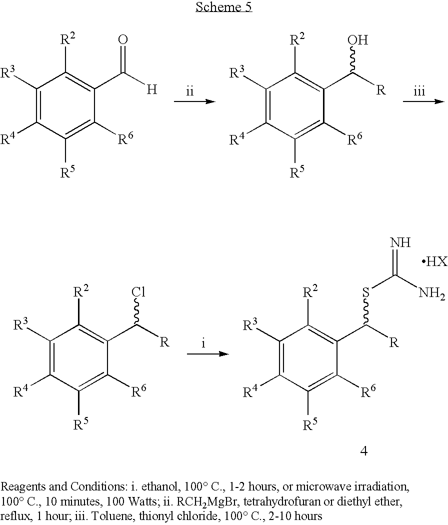

Benzylguanidinium salts 6 may be obtained via the reaction between di-tert-butoxycarbonyl thiourea and the appropriate benzylamine, (Yong 1997) followed by deprotection of the corresponding di-tert-butoxycarbonyl guanidine product with tin(IV) chloride (Miel 1997) or trifluoroacetic acid, (Guisado 2002).

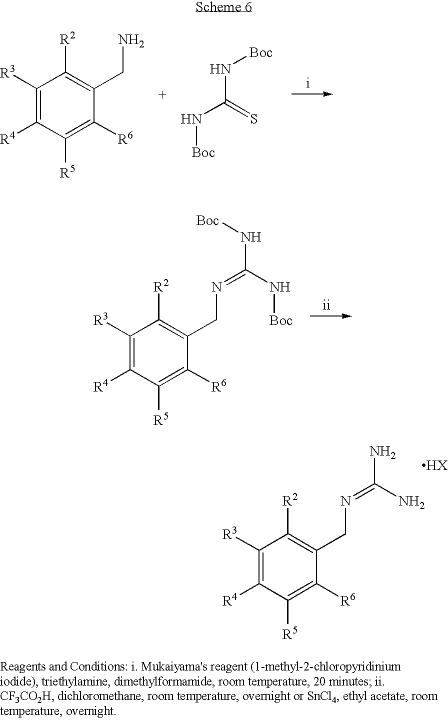

### Typical Reaction Conditions for Synthesis of Compounds 3, 10 and 11.

A microwave reaction tube (2 mL) is charged with a mixture of ethanol (0.5-1 mL), the appropriate benzyl chloride (1-2 mmol) and thiourea or guanylthiourea (1 molar eq.). The tube is capped and heated in a microwave reactor (Biotage Initiator I) at 110-120° C. for 30-45 minutes. The reactions are monitored by thin layer chromatography (ethyl acetate:hexane, 1:4, v:v). After the reaction is complete, the reaction mixture was concentrated under vacuum and the residue is washed with hexane. The solid product is filtered and dried under high vacuum to give the product.

### Typical Reaction Conditions for Synthesis of Compounds 3.

A 10 milliliter (mL) microwave reaction tube is charged with the benzyl halide (1.0 millimole, mmol) and thiourea (76 mg, 1.0 mmol) in ethanol (1.5 mL). The tube is capped and irradiated in the microwave reactor (single-mode CEM Discover™ system, CEM, Matthews, N.C.) at 100° C. for 15 minutes. The solid is filtered and solid washed with cold ethanol. The solid product is dried under high vacuum to give the product.

The following compounds were prepared by the foregoing methods:

### Example 1

### (2,4-Dichlorophenyl)methyl Isothiourea Hydrochloride (3a)

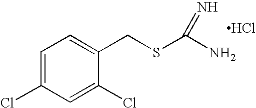

White solid, mp 222-223° C.; ¹H NMR (400 MHz, d₆-DMSO) δ 4.58 (s, 2H), 7.47 (dd, J=8.0 and 2.0 Hz, 1H), 7.63 (d, J=8.0 Hz, 1H), 7.70 (d, J=2.0 Hz, 1H), 9.31 (br s, 2H), 9.39 (br s, 2H); ¹³C NMR (100 MHz, d₆-DMSO) δ 32.6, 128.5, 130.0, 132.5, 133.3, 134.5, 135.1, 169.4; MS (ESI) m/z 235.0 (100%, [M+H]⁺); HRMS calcd for C₈H₉Cl₂N₂S: 234.9858; observed: 234.9854; HPLC analysis (Alltech C18): 90% methanol, 10% acetonitrile, flow rate 0.5 mL/min: t_{R} 3.26 min. 90% acetonitrile, 10% water, flow rate 0.75 mL/min: t_{R} 2.05 min. 100% methanol, flow rate 0.5 mL/min: t_{R} 3.05 min.

### Example 2

### (4-Chloro-2-nitrophenyl)methyl Isothiourea Hydrochloride (3u)

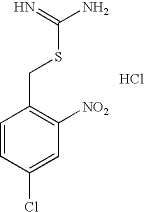

White solid, 44%. ¹H NMR (400 MHz, DMSO-d₆) δ 4.72 (s, 2H), 7.75 (d, J=8.4 Hz, 1H), 7.90 (dd, J=8.4, 2.2 Hz, 1H), 8.22 (d, J=2.2 Hz, 1H), 9.22 (bs, 4H); HRMS calcd. for C₈H₈ClN₃O₂S (M-Cl)⁺ 246.00985, found 246.01283.

### Example 3

### 2-Chloro-4-fluorophenyl)methyl isothiourea Hydrochloride (3v)

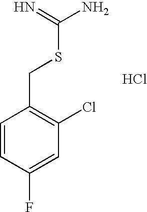

White solid, 100%. ¹H NMR (400 MHz, DMSO-d₆) δ 4.57 (s), 7.28 (td, J=8.4, 2.4 Hz, 1H), 7.55 (dd, J=8.6, 2.4 Hz, 1H), 7.66 (dd, J=8.4, 6.2 Hz, 1H), 9.29 (bs, 4H); HRMS calcd. for C₈H₈ClFN₂S (M-Cl)⁺219.01535, found 219.01549.

### Example 4

### (2,4-Difluorophenyl)methyl isothiourea Hydrochloride (3w)

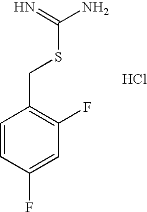

White solid, 100%. ¹H NMR (400 MHz, DMSO-d₆) δ 4.55 (s, 2H), 7.14 (t, J=8.1 Hz, 1H), 7.34 (t, J=9.8 Hz, 1H), 7.60 (q, J=7.9 Hz, 1H), 9.30 (bs, 2H) 9.37 (bs, 2H); HRMS calcd. for C₈H₈ClN₃O₂S (M−

### Example 5

### (2-Chloro-4-fluorophenyl)methyl Amidinoisothiourea Hydrochloride (10a)

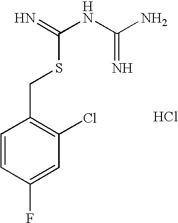

White solid, 75%; m.p. 154-156° C.; ¹H NMR (400 MHz, DMSO-d₆) δ 4.28 (s, 2H), 7.20 (td, J=8.5, 2.6 Hz, 1H), 7.48 (dd, J=8.8, 2.6 Hz, 1H), 7.57 (dd, J=8.7, 6.24 Hz, 1H), 8.00 (bs, 4H), 8.10 (s, 2H); HRMS calcd. for C₉H₁₁ClFN₄S (M-Cl)⁺261.03715, found 261.03737.

### Example 6

### (2,4-Difluorophenyl)methyl Amidinoisothiourea Hydrochloride (10b)

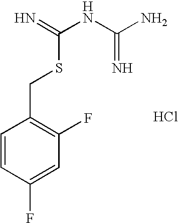

White solid, 78%; m.p. 144-146° C.; ¹H NMR (400 MHz, DMSO-d₆) δ 4.22 (s, 2H), 7.06 (td, J=8.6, 2.3 Hz, 1H), 7.25 (td, J=9.8, 2.4 Hz, 1H), 7.51 (td, J=8.6, 6.2 Hz, 1H), 7.98 (bs, 4H), 8.09 (s, 2H); HRMS calcd. for C₉H₁₁F₂N₄S (M-Cl)⁺245.06670, found 245.06731.

### Example 7

### (2,4-Dichlorophenyl)methyl Amidinoisothiourea Hydrochloride (10c)

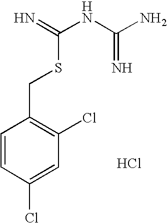

White solid, 74%; m.p. 139-142° C. ¹H NMR (400 MHz, CD₃OD) δ 4.34 (s, 2H), 7.30 (dd, J=8.3, 2.1 Hz, 1H), 7.47 (d, J=2.1 Hz, 1H), 7.50 (d, J=8.3 Hz, 1H), HRMS calcd. for C₉H₁₁Cl₂N₄S (M-Cl)⁺277.00760, found 277.00741.

### Example 8

### (2-Nitro-4-chlorophenyl)methyl Amidinoisothiourea Hydrochloride (10d)

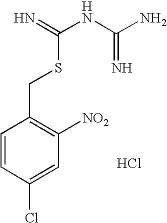

Off-white solid, 28%; m.p. 183-185° C. ¹H NMR (400 MHz, CD₃OD) δ 4.52 (s, 2H), 7.66-7.72 (m, 2H), 8.06 (s, 1H); HRMS calcd. for C₉H₁₀ClN₃O₂S (M-Cl)⁺288.03165, found 288.03168.

### Example 9

### (4-Cyanophenyl)methyl Amidinoisothiourea Hydrochloride (10e)

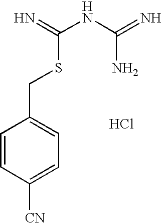

White solid, 42%. ¹H NMR (400 MHz DMSO-d₆) δ 8.05 (bs, 4H), 7.78 (d, 2H, J=8.2 Hz), 7.71 (bs, 1H), 7.55 (d, 2H, J=8.1 Hz), 4.27 (s, 2H); HRMS calcd. for C₁₀H₁₂N₅S (M-Cl)⁺ 234.08079, found 234.08155.

### Example 10

### (2,5-Dichlorophenyl)methyl Amidinoisothiourea Hydrochloride (10f)

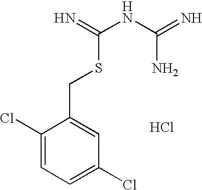

White solid, 47%. ¹H NMR (400 MHz DMSO-d₆) δ 8.10 (bs, 6H), 7.56 (d, 1H, J=2.5 Hz), 7.50 (d, 1H, J=8.6 Hz), 7.40 (dd, 1H, J=2.6, 8.6 Hz), 4.27 (s, 2H); HRMS calcd. for C₉H₁₁Cl₂N₄S (M-Cl)⁺277.00760, found 277.00839.

### Example 11

### (2-Chloro-6-fluorophenyl)methyl Amidinoisothiourea Hydrochloride (10 g)

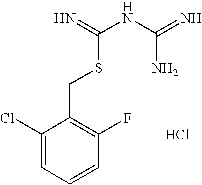

White solid, 57%. ¹H NMR (400 MHz DMSO-d₆) δ 8.08 (bs, 4H), 7.85 (bs, 2H), 7.43-7.35 (m, 2H), 7.28-7.23 (m, 1H), 4.33 (s, 2H); HRMS calcd. for C₉H₁₁ClFN₄S (M-Cl)⁺ 261.03807, found 261.03801.

### Example 12

### (6-Chlorobenzo[d][1,3-dioxol-5-yl)methyl Amidinoisothiourea Hydrochloride (10h)

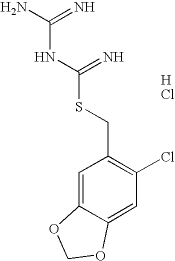

White solid, 67%. ¹H NMR (400 MHz, CD₃OD) δ 6.97 (s, 1H), 6.90 (s, 1H), 5.98 (s, 2H), 4.28 (s, 2H); HRMS calcd. for C₁₀H₁₂ClFN₄O₂S (M-Cl)⁺ 287.03640, found 287.04802.

### Example 13

### (4-Chloro-3-fluorophenyl)methyl Amidinoisothiourea Hydrochloride (10i)

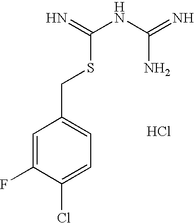

White solid, 45%. ¹H NMR (400 MHz DMSO-d₆) δ 8.07-7.89 (m, 6H), 7.51 (t, 1H, J=8.1 Hz), 7.39 (dd, 1H, J=1.8, 10.4 Hz), 7.22 (dd, 1H, J=1.8, 8.3 Hz), 4.21 (s, 2H); HRMS calcd. for C₉H₁₁ClN₄S (M-Cl)⁺ 261.03715, found 261.03706.

### Example 14

### (2,6-Difluorophenyl)methyl Amidinoisothiourea Hydrochloride (10j)

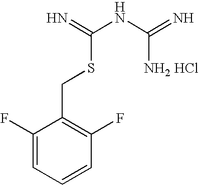

White solid, 53%. ¹H NMR (400 MHz, DMSO-d₆) δ 8.06 (s, 4H), 7.82 (s, 2H), 7.46-7.38 (m, 1H), 7.12 (t, 2H, J=8.1 Hz), 4.24 (s, 2H); HRMS calcd. for C₉H₁₁F₂NO₂S (M-Cl)⁺ 245.06670, found 245.06687.

### Example 15

### (2-Naphthyl)methyl Amidinoisothiourea hydrochloride (11a)

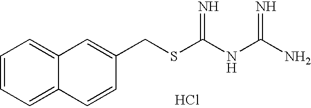

White solid, 80%; m.p. 175-177° C. ¹H NMR (400 MHz, DMSO-d₆) δ 4.39 (s, 2H), 7.49-7.52 (m, 3H), 7.85-7.89 (m, 4H), 8.08 (bs, 1H), 9.37 (bs, 3H, disappeared on D₂O shake); HRMS calcd. for C₁₃H₁₄BrN₄S (M-Cl)⁺ 259.10119, found 259.10063.

### Example 16

### 2-(1-Bromonaphthyl)methyl amidinoisothiourea hydrochloride (11b)

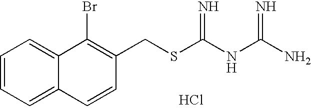

White solid, 62%; m.p. 160-162° C. ¹H NMR (400 MHz, DMSO-d₆) δ 4.53 (s, 2H), 7.61 (m, 2H), 7.67-7.71 (m, 1H), 7.93 (bs, 1H, disappeared on D₂O shake), 7.94 (d, J=8.5 Hz, 1H), 7.98 (d, J=8.1 Hz, 1H), 8.13 (bs, 3H, disappeared on D₂O shake), 8.20 (d, J=8.5 Hz, 1H); HRMS calcd. for C₁₃H₁₄BrN₄S (M-Cl)⁺ 337.01171, found 337.01251.

### Rb:Raf-1 Binding Inhibition Activity for the Example Compounds

The compounds were screened for Rb:Raf-1 binding inhibitory properties using a GST-Rb/GST-Raf-1 ELISA assay. The results are reported as inhibition of Rb:Raf-1 binding at a concentration of 10 or 20 micromolar (μM, Tables 1-4). The compounds can be further evaluated by generating a dose response for the most active compounds—those that inhibit the interaction by 80% or greater at 20 μM to generate an IC₅₀ value.

The most active compounds tended to possess a monosubstituted or disubstituted benzene ring, bearing at least one halide in either one or both of the positions ortho, meta, or para to the carbon bound to the isothiouronium group.

TABLE 1
Structures, yields of compounds 3a-z, and
inhibition of Rb:Raf-1 binding.

| | |
|---|---|
| | 3 |
| 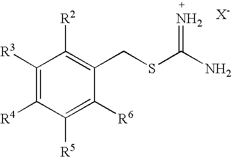 | |

| | | | | | | | | % Inhibition at |
|---|---|---|---|---|---|---|---|---|
| Compound | R² | R³ | R⁴ | R⁵ | R⁶ | X | Yield (%) | 10 or 20 μM |
| 3a | Cl | H | Cl | H | H | Cl | 98 | 100 (at 20 μM) |
| 3u | NO₂ | H | Cl | H | H | Cl | 44 | + |
| 3v | Cl | H | F | H | H | Cl | 100 | ++ |
| 3w | F | H | F | H | H | Cl | 100 | ++ |

+ signifies 25-50% inhibition at 10 μM;
++ signifies 50-100% inhibition at 10 μM

TABLE 2
Structures of compounds 10a-d, yields, and
inhibition of Rb:Raf-1 binding.

| | |
|---|---|
| | 10 |
| 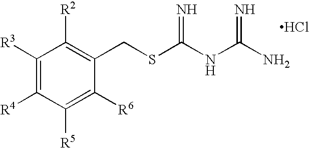 | |

| | | | | | | | Inhibition at 10 |
|---|---|---|---|---|---|---|---|
| Compound | R² | R³ | R⁴ | R⁵ | R⁶ | Yield (%) | μM or 20 μM |
| 10a | Cl | H | F | H | H | 75 | ** |
| 10b | F | H | F | H | H | 78 | ** |
| 10c | Cl | H | Cl | H | H | 74 | ** |
| 10d | NO₂ | H | Cl | H | H | 28 | + |
| 10e | H | H | CN | H | H | 42 | 6%, 22% at |
| | | | | | | | 20 μM |
| 10f | Cl | H | H | Cl | H | 47 | ** |
| 10g | Cl | H | H | H | F | 57 | ** |
| 10h | Cl | H | —OCH₂O— | | H | 67 | ** |
| 10i | H | F | Cl | H | H | 45 | 6%, 42% at |
| | | | | | | | 20 μM |
| 10j | F | H | H | H | F | 53 | ** |

+ signifies 25-50% inhibition at 10 μM;
** signifies 50-100% inhibition at 20 μM

TABLE 3
Structures of compounds 11a-b, yields, and
inhibition of Rb:Raf-1 binding.

| | |
|---|---|
| | 11a-b |
| 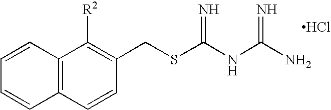 | |

| Compound | R² | Yield (%) | Inhibition at 10 μM |
|---|---|---|---|
| 11a | H | 80 | ++ |
| 11b | Br | 62 | ++ |

+ signifies 25-50% inhibition at 10 μM;
++ signifies 50-100% inhibition at 10 μM

### Example 17

### Modulators of Rb:Raf 1 Interactions Disrupt Rb:Raf-1 in Intact Cells

U937 cells were serum starved serum starved for 48 hours and subsequently serum stimulated for 2 hours in the presence or absence of 20 μM of the compounds. Compounds 10b and 10c significantly inhibited the binding of Raf-1 to Rb, as seen by immunoprecipitation-Western blot analysis (FIG. 1A). Raf-1 peptide conjugated to penetratin was used as a positive control. Thus it appears that these two compounds were capable of disrupting the Rb:Raf-1 interaction.

### Example 18

### Compounds 10b & 10c Inhibited Epithelial Lung Cancer Cells

Compounds 10b and 10c inhibited the proliferation of epithelial lung cancer cells. To investigate whether compounds 10b and 10c require a functional Rb to inhibit tumor cell proliferation, A549 cells (human epithelial lung carcinoma) were stably transfected with two different shRNA constructs (sh6 and sh8) to knock down Rb expression (FIGS. 1B and 1C). A549 cells stably expressing the Rb shRNAs had significantly less Rb protein compared to parental A549 cells. Compounds 10b and 10c were very effective at inhibiting S-phase entry in parental A549 cells but had little or no effect on cells stably expressing sh6 and sh8, which lacked Rb. This result confirms that compounds 10b and 10c arrest the proliferation of epithelial lung cancer cells in a Rb dependent manner.

### Example 19

### Dose-Dependent Inhibition of Cancer Cells by 3w, 10a, 10b and 10c

Compounds 3w, 10a, 10b and 10c inhibited the proliferation of epithelial lung cancer cells in a dose-dependent manner. Similar to the preceding example, A549 cells (human epithelial lung carcinoma) were contacted with compounds 3w, 10a, 10b and 10c (FIG. 1D). A BrdU incorporation assay at compound concentrations of 5, 10, 20, 30 and 50 μM shows dose-dependent inhibition of wild-type A549 cells by compounds 3w, 10a, 10b and 10c. This result confirms that compounds 3w, 10a, 10b and 10c arrest the proliferation of epithelial lung cancer cells.

### Example 20

### Modulators of Rb:Raf 1 Interactions Disrupt Angiogenesis

An experiment was performed to determine whether angiogenic tubule formation could be inhibited by compounds 10b and 10c. Human aortic endothelial cells (HAECs) were grown in matrigel in the presence or absence of 20, 50 and 100 μM of 10b or 10c, or 100 μM of compound 3a. It was found that while angiogenic tubules formed in control (no drug) wells, compounds 10b and 10c significantly inhibited angiogenic tubule formation in a dose dependent fashion, and showed inhibition comparable to that of compound 3a at 100 μM (FIG. 1E).

### Example 21

### Modulators of Rb:Raf 1 Interactions 3a & 9a Significantly Inhibited Human Tumor Line In Vivo

Experiments were performed to assess whether compounds 10b and 10c could inhibit human tumor growth in vivo using a nude mice xenograft model. Athymic nude mice were implanted with 1×10⁷ A549 cells bilaterally and the tumors were allowed to reach 200 mm³ in size before treatment began. FIG. 1F shows that tumors from vehicle treated mice grew to an average size of over 1200 mm³. In contrast, tumors treated with compounds 10b and 10c at 150 mg/kg were substantially inhibited.

### Example 22

### Compound 10c Inhibited 7 Disparate Human Cancer Cell Lines

Compound 10c inhibited the proliferation of a wide range of cancer cells at 20 μM as shown in FIG. 1G. In a BrdU incorporation assay, compound 10c was contacted with a range of cancer cells including PANC-1 (human pancreatic carcinoma, epithelial-like), CAPAN-2 (human pancreatic ductal adenocarcinoma), MeI-5 (human malignant melanoma), MCF-7 (human breast adenocarcinoma), LNCAP (androgen-sensitive human prostate adenocarcinoma), A549 (human epithelial lung carcinoma), and PC-3 (human prostate adenocarcinoma), and compared to Rb-deficient cancer cells (A549 cells stably transfected with two different shRNA constructs (sh6 and sh8) to knock down Rb expression, and the Rb-deficient prostate cancer cell line DU145). This result confirms that compound 10c arrests the proliferation of a wide variety of cancer cells in a Rb dependent manner.

### Example 23

### Compounds 3a, 10b and 10c Reduce the Viability of U937 Myeloid Cells

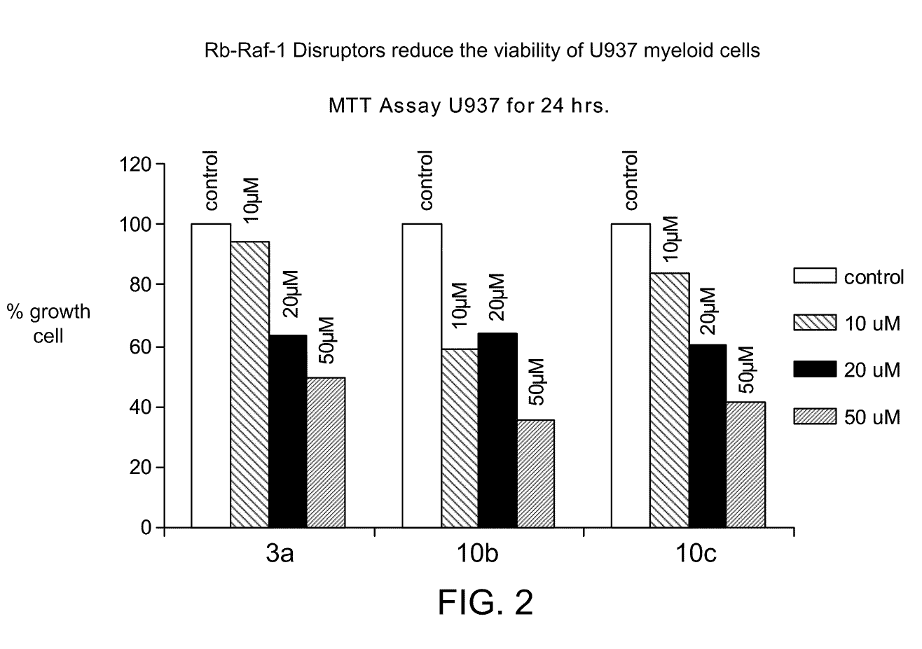

U937 myeloid cells were incubated in the absence of compound (control), or with compounds 3a, 10b, or 10c at 10 μM, 20 μM, or 50 μM for 24 hours. Cell viability was assessed by an MTT assay, a colorometric assay which measures the number of cells by measuring the activity of enzymes that reduce 3-(4,5-dimethylthiazol-2-yl)-2,5-diphenyltetrazolium bromide. The results are shown in FIG. 2. A dose-dependent reduction in cell number was seen with each of the compounds, demonstrating that they to reduce cell viability significantly.

### Example 24

### Compounds 3a, 10b and 10c Reduce the Viability of Ramos Burkitt's Lymphoma Cells

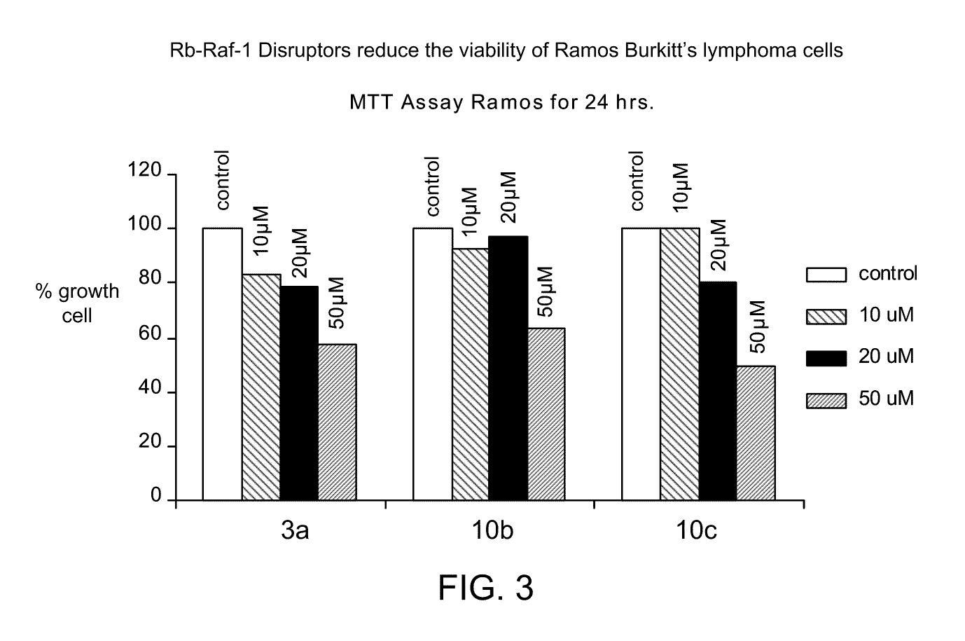

Ramos cells (Burkitt's Lymphoma) were incubated in the absence of compound (control), or with compounds 3a, 10b, or 10c at 10 μM, 20 μM, or 50 μM for 24 hours. Cell viability was assessed by an MTT assay, a colorometric assay which measures the number of cells by measuring the activity of enzymes that reduce 3-(4,5-dimethylthiazol-2-yl)-2,5-diphenyltetrazolium bromide. The results are shown in FIG. 3. A dose-dependent reduction in cell number was seen with each of the compounds, demonstrating that they reduce cell viability significantly.

### Example 25

### Evidence that Inhibition of Cell Proliferation by Compounds of the Invention is Mediated by Raf-1

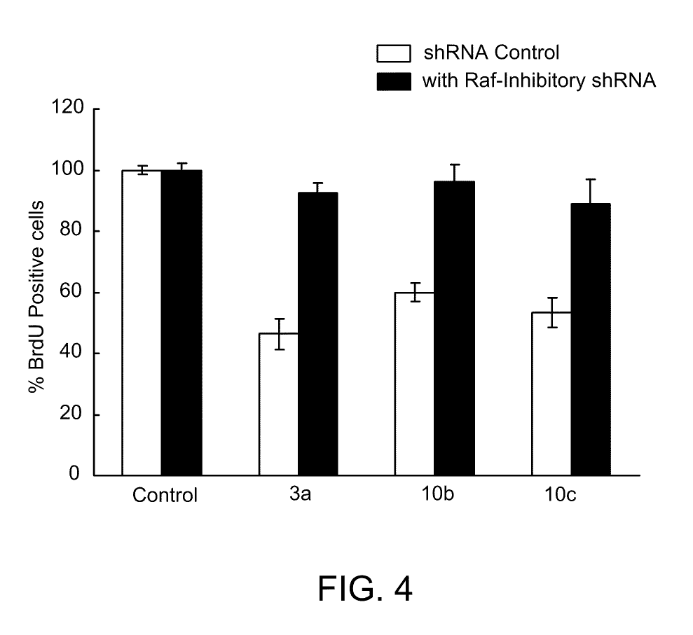

A549 cells lacking Raf-1 (sh-13B) were generated by stably transfecting a shRNA to Raf-1. Control cells were generated by stably transfecting A549 cells with a control shRNA. The cells were incubated in the presence or absence of compounds 3a, 10b and 10c (20 μM) and S-phase entry was assessed by measuring BrdU incorporation. The results are shown in FIG. 4. Relative to controls incubated in the absence of compound, proliferation of the cells with control shRNA (having Raf-1) was inhibited by each of the compounds. In contrast, proliferation of the cells lacking Raf-1 (the cells transfected with Raf-inhibitory shRNA) was not inhibited by the compound. This experiment provides evidence that inhibition of cell proliferation by compounds of the invention is mediated by Raf-1 as well as Rb and Raf-1.

### Example 26

### Evidence that the Rb-E2F Pathway Regulates the Expression of Matrix Metalloproteinase (MMP) Genes

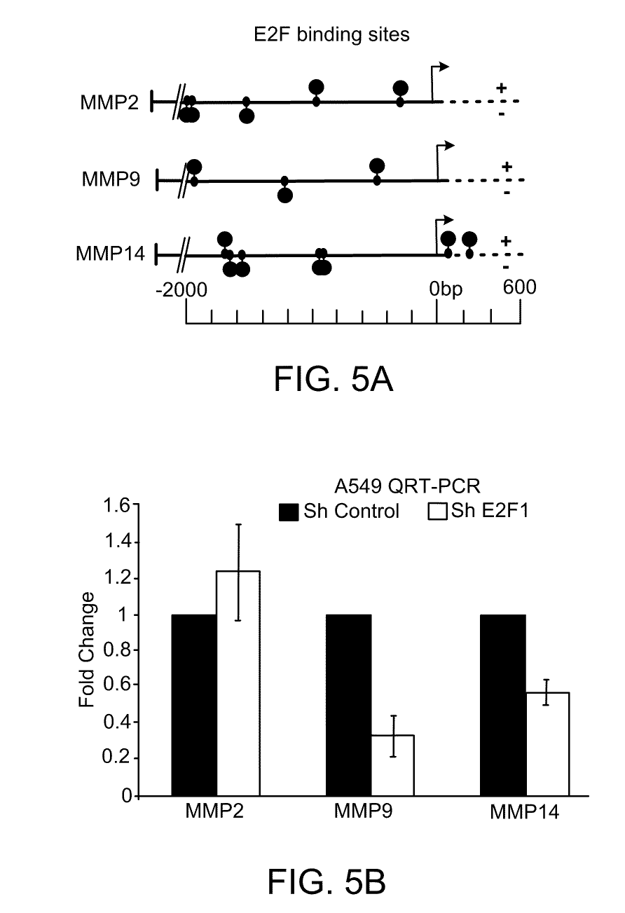

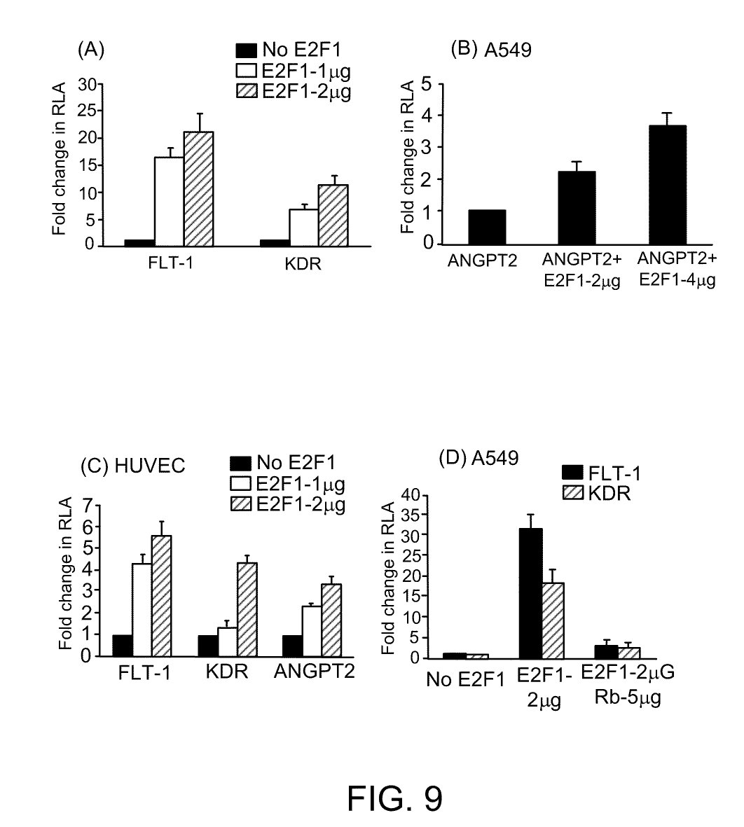

FIG. 9A shows a schematic of the promoters showing the E2F binding site on the genes for MMP2, MMP9 and MMP14. Using A549 cells transfected with an shRNA to inhibit expression of E2F1, QRT-PCR experiments were performed to measure the expression of matrix metalloproteinases, MMP2, MMP9 and MMP14. The results are shown in FIG. 5 and show that when A549 cells are depleted of E2F1, the expression of MMP9 and MMP14 is reduced. This experiment provides evidence that the Rb-E2F pathway can regulate the expression of matrix metalloproteinases (MMPs).

### Example 27

### Immunoprecipitation Assays Showing that Rb and E2F1 Associate with MMP Promoters

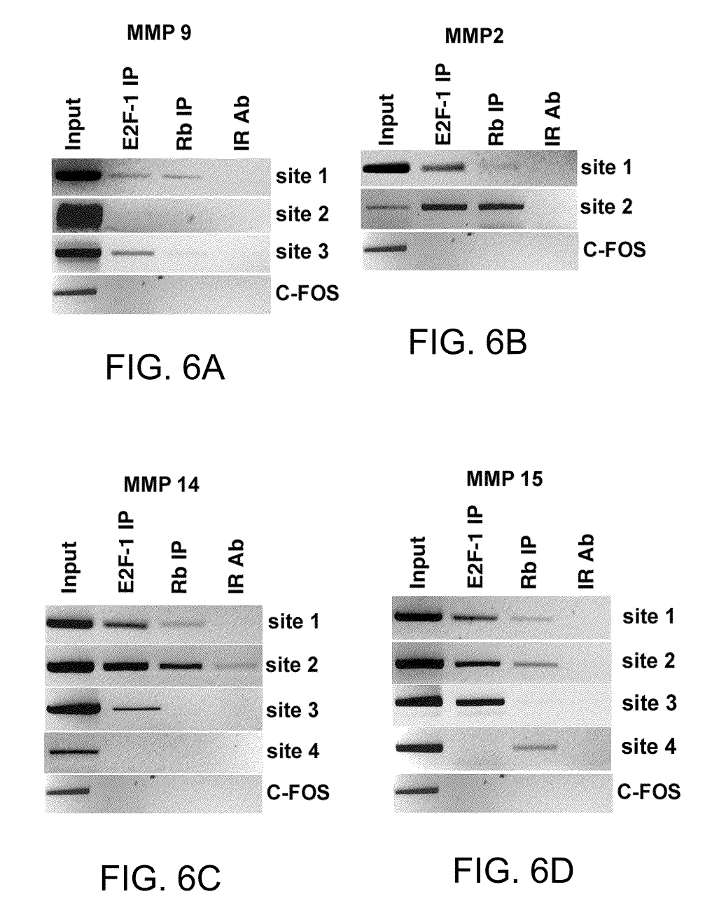

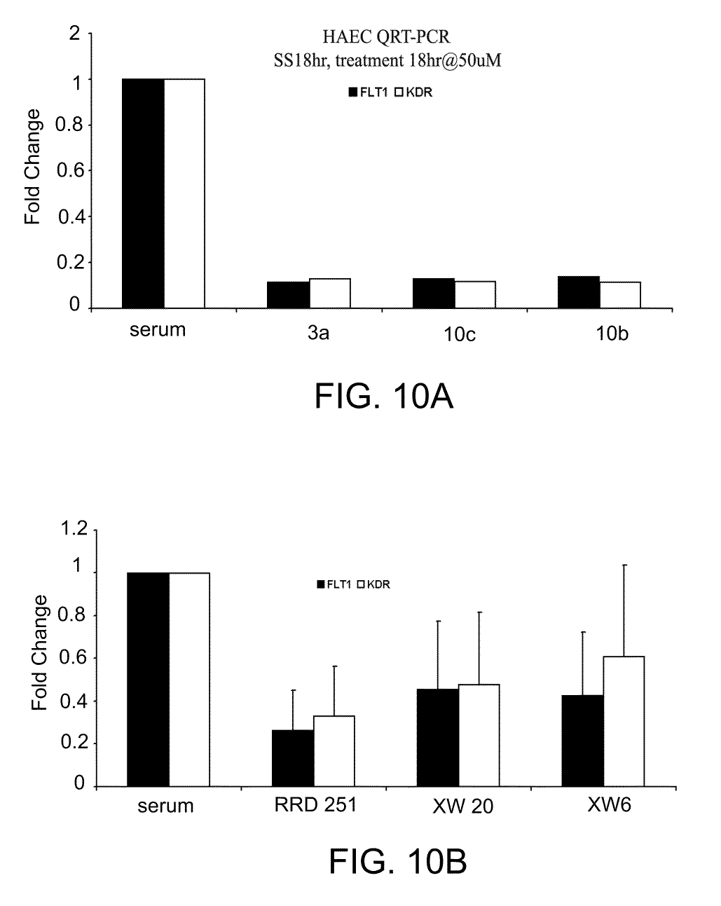

FIG. 10 shows the results of chromatin immunoprecipitation assays showing the binding of E2F1 as well as the association of Rb with the promoters of matrix proteases. Experiments were performed with respect to MMP9 (FIG. 6A), MMP2 (FIG. 6B), MMP14 (FIG. 6C), and MMP15 (FIG. 6D). This is as assay used to assess the binding of proteins to promoters in living cells. These results provide evidence that E2F1 is associated with these promoters in the cells, regulating their expression.

### Example 28

### Evidence that Compounds of the Invention Inhibit Expression of Matrix Metalloproteinases MMP9, MMP14 and MMP15

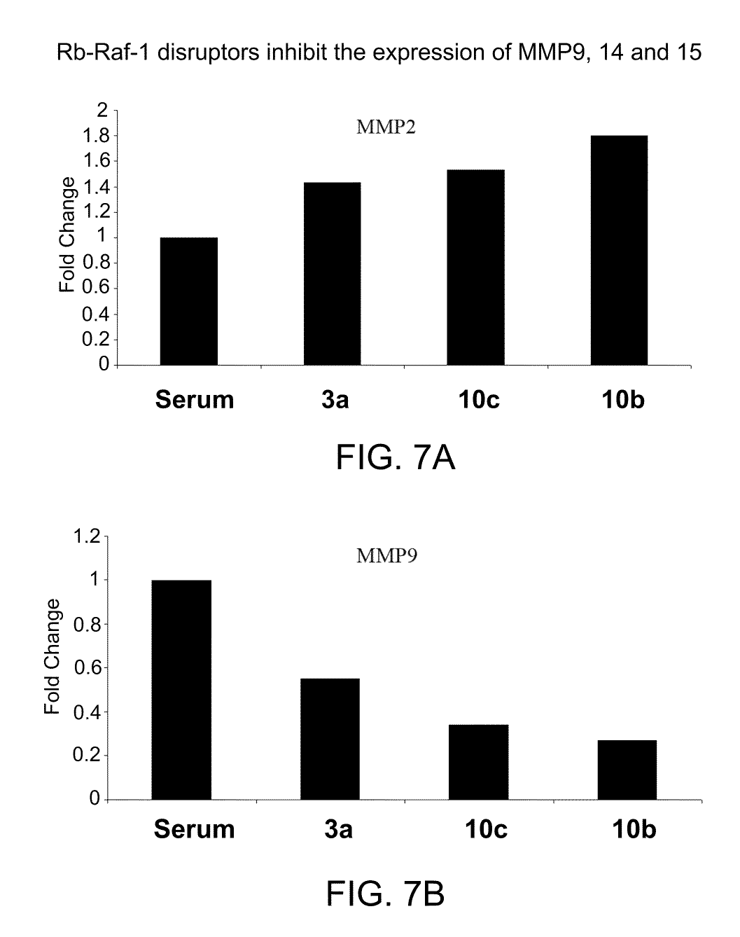

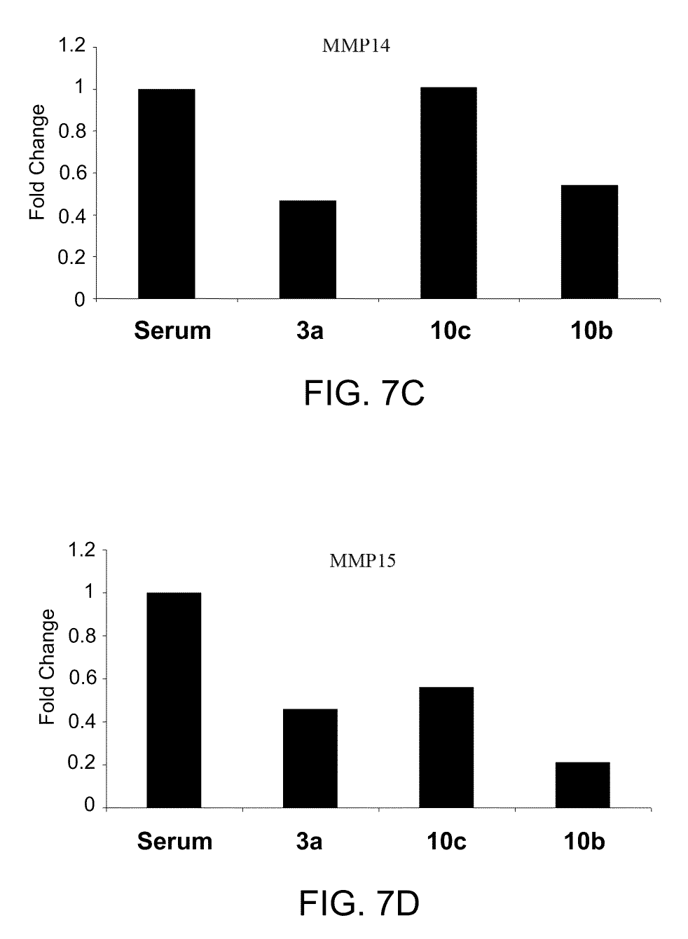

A quantitative real-time PCR experiment was performed to measure the effect of compounds 3a, 10b and 10c on the expression of MMP2, MMP9, MMP14 and MMP15 in MDAMB231 cells (breast cancer). The cells were incubated either in the absence of compound or in the presence of compound (50 μM) for 24 hours. The results are shown in FIGS. 7A (MMP2), 7B (MMP9), 7C (MMP14) and 7D (MMP15). Expression of MMP9, MMP14 and MMP15 was inhibited by each of the compounds. These results provide evidence that the compounds of the invention are effective in controlling the expression of genes that are involved in metastasis.

### Example 29

### Evidence that Rb and E2F Associate with and Induce FLT1 and KDR Promoters

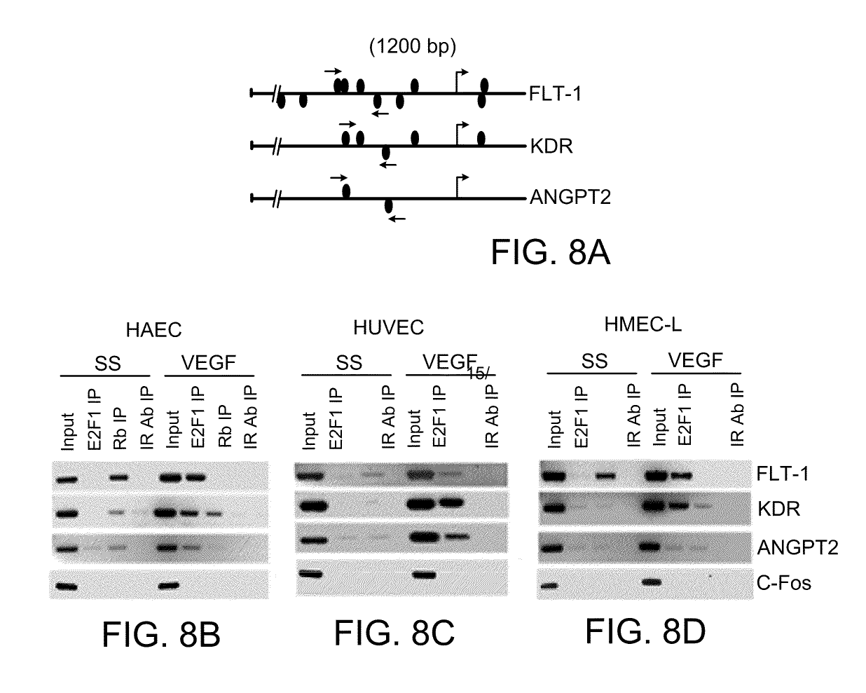

The data shown in FIG. 12 promoters for VEGF receptors, FLT1 and KDR, have E2F binding sites, shown schematically in FIG. 8A. FIGS. 8B-D show the results of chromatin immunoprecipitation assay performed using primary endothelial cells: human aortic endothelial cells HAEC (FIG. 8B), human umbilical cord vein endothelial cell (HUVEC) (FIG. 8C) and human microvascular endothelial cells from the lung (HMEC-L) (FIG. 8D). Treatment of the primary endothelial cells (human aortic endothelial cells, human umbilical cord vein endothelial cells or human microvascular endothelial cells from the lung) with VEGF induced the binding of E2F1 to the FLT1 and KDR promoters. This provides evidence that these promoters can be regulated by the Rb-E2F pathway and could possibly be targeted by the Rb-Raf-1 disruptors.

The data shown in FIG. 9 demonstrates transient transfection of E2F1 induces FLT1 and KDR promoters and that Rb can repress these promoters. The transfection assays were performed in both A549 and HUVEC cells.

### Example 30

### Evidence that Compounds of the Invention Inhibit the Expression of FLT1 and KDR

A quantitative real-time PCR experiment was performed to measure the effect of compounds 3a, 10b and 10c on the expression of FLT1 and KDR in human aortic endothelial cells. The cells were incubated either in the absence of compound or in the presence of compound (50 μM) for 18 hours. The results are shown in FIG. 10. Each of the compounds inhibits expression of both FLT and KDR. These results provide evidence that the compounds of the invention inhibit the expression of FLT and KDR.

### REFERENCES

The entire teachings of each document cited herein, including each of the following, are incorporated by reference.
Alavi A, Hood J D, Frausto R, Stupack D G, Cheresh D A. Role of Raf in vascular protection from distinct apoptotic stimuli. Science 2003, 301(5629), 94-96.
Arkin M R, Wells J A. Small-molecule inhibitors of protein-protein interactions: progressing towards the dream. Nat. Rev. Drug Discov. 2004, 3(4), 301-317.
Bagchi S, Weinmann R, Raychaudhuri P. The retinoblastoma protein copurifies with E2F-I, an E1A-regulated inhibitor of the transcription factor E2F. Cell 1991; 65(6):1073-82.
Beijersbergen R L, Bernards R. Cell cycle regulation by the retinoblastoma family of growth inhibitory proteins. Biochim Biophys Acta 1996; 1287(2-3):103-20.
Chau B N, Wang J Y. Coordinated regulation of life and death by RB. Nat Rev Cancer 2003; 3(2): 130-8.
Chellappan S, Kraus V B, Kroger B, et al. Adenovirus E1A, simian virus 40 tumor antigen, and human papillomavirus E7 protein share the capacity to disrupt the interaction between transcription factor E2F and the retinoblastoma gene product. Proc Natl Acad Sci USA 1992; 89:4549-53.
Chellappan S P, Hiebert S, Mudryj M, Horowitz J M, Nevins J R. The E2F transcription factor is a cellular target for the RB protein. Cell 1991; 65(6):1053-61.
Chittenden T, M. L D, Jr KWG. RB associates with an E2F-like, sequence-specific DNA-binding protein. Cold Spring Harb Symp Quant Biol 1991; 56:187-95.
Classon M, Dyson N. p107 and p130: versatile proteins with interesting pockets. Exp Cell Res 2001; 264(1):135-47.
Classon M, Salama S, Gorka C, Mulloy R, Braun P, Harlow E. Combinatorial roles for pRB, p107, and p130 in E2F-mediated cell cycle control. Proc Natl Acad Sci USA 2000; 97(20):10820-5.
Cobrinik D. Pocket proteins and cell cycle control. Oncogene 2005, 24(17), 2796-2809.
Dasgupta P, Betts V, Rastogi S, et al. Direct binding of apoptosis signal regulating kinase 1 to retinoblastoma protein: novel links between apoptotic signaling and cell cycle machinery. J. Biol. Chem. 2004; 279(37), 38762-38769.
Dasgupta P, Betts V, Rastogi S, et al. Direct binding of apoptosis signal-regulating kinase 1 to retinoblastoma protein: novel links between apoptotic signaling and cell cycle machinery. J Biol Chem 2004; 279(37):38762-9.
Dasgupta P, Chellappan S. Nicotine-mediated cell proliferation and angiogenesis: New twists to an old story. Cell Cycle 2006, in press.
Dasgupta, P, Rastogi, S, Pillai, S, Ordonez-Ercan, D, Morris, M. at al. Nicotine induces cell proliferation by beta-arrestin-mediated activation of Src and Rb:Raf-1 pathways. J. Clin. Invest. 2006, 116, 2208-2217.
Dasgupta, P, Sun, J Z, Wang, S, Fusaro, G, Betts, V. at al. Disruption of the Rb:Raf-1 interaction inhibits tumor growth and angiogenesis. Mol. Cell. Biol. 2004, 24, 9527-9541.
de Bruin A, Maiti B, Jakoi L, Timmers C, Buerki R, Leone G. Identification and characterization of E2F7, a novel mammalian E2F family member capable of blocking cellular proliferation. J Biol Chem 2003; 278(43):42041-9.
DeGregori J, Leone G, Miron A, Jakoi L, Nevins J R. Distinct roles for E2F proteins in cell growth control and apoptosis. Proc Nail Acad Sci USA 1997; 94(14):7245-50.
DeGregori J, Leone G, Ohtani K, Miron A, Nevins J R. E2F-1 accumulation bypasses a G1 arrest resulting from the inhibition of G1 cyclin-dependent kinase activity. Genes Dev 1995; 9(23):2873-87.
Derossi D, Chassaing G, Prochiantz A. Trojan peptides: the penetratin system for intracellular delivery. Trends Cell Biol 1998; 8(2):84-7.
Derossi, D, Joliot, A H, Chassaing, G, Prochiantz, A. The 3rd Helix of the Antennapedia Homeodomain Translocates through Biological-Membranes. J. Biol. Chem. 1994, 269, 10444-10450.
Di Stefano L, Jensen M R, Helin K. E2F7, a novel E2F featuring DP-independent repression of a subset of E2F-regulated genes. Embo J 2003; 22(23):6289-98.
Dyson N, Guida P, McCall C, E. H. Adenovirus E1A makes two distinct contacts with the retinoblastoma protein. J Virol 1992; 66(7):4606-11.
Dyson N, Howley P M, Munger K, Harlow E. The human papilloma virus-16 E7 oncoprotein is able to bind to the retinoblastoma gene product. Science 1989; 243(4893):934-7.
Guisado, O, Martinez, S, Pastor, J. A novel, facile methodology for the synthesis of N,N′-bis(tert-butoxycarbonyl)-protected guanidines using polymer-supported carbodiimide. Tetrahedron Lett. 2002, 43, 7105-7109.
Hakem R, Mak T W. Animal models of tumor-suppressor genes. Ann Rev Genet 2001; 35:209-41.
Harbour J W, Luo R X, Dei Santi A, Postigo A A, Dean D C. Cdk phosphorylation triggers sequential intramolecular interactions that progressively block Rb functions as cells move through G1. Cell 1999, 98(6), 859-869.
Harbour, J W, Dean, D C. Rb function in cell-cycle regulation and apoptosis. Nat. Cell Biol. 2000, 2, E65-E67.
Harbour, J W, Dean, D C. The Rb/E2F pathway: expanding roles and emerging paradigms. Genes Dev. 2000, 14, 2393-2409.
Hood J D, Cheresh D A. Targeted delivery, of mutant Raf kinase to neovessels causes tumor regression. Cold Spring Harbor Symp. on Quant. Biol. 2002, 67, 285-291.
Ishida S, Huang E, Zuzan H, et al. Role for E2F in control of both DNA replication and mitotic functions as revealed from DNA microarray analysis. Mol Cell Biol 2001; 21(14):4684-99.
Johnson D G, Schneider-Broussard R. Role of E2F in cell cycle control and cancer. Front Biosci 1998; 3:d447-8.
Johnson D G, Schwarz, J. K., Cress, W. D. and Nevins, J. R. Expression of transcription factor E2F1 induces quiescent cells to enter S phase. Nature 1993; 365:349-52.
Kasid, U. Raf-1 protein kinase, signal transduction, and targeted intervention of radiation response. Experimental Biology and Medicine 2001, 226, 624-625.
Kato J, Matsushime H, Hiebert S W, Ewen M E, Sherr C J. Direct binding of cyclin D to the retinoblastoma gene product (pRb) and pRb phosphorylation by the cyclin D-dependent kinase CDK4. Genes Dev 1993; 7:331-42.

Knudsen E S, Wang J Y. Differential regulation of retinoblastoma protein function by specific Cdk phosphorylation sites. J. Biol. Chem. 1996, 271(14), 8313-8320.
Knudsen E S, Wang J Y. Dual mechanisms for the inhibition of E2F binding to RB by cyclin-dependent kinase-mediated RB phosphorylation. Mol. Cell. Biol. 1997, 17(10), 5771-5783.
Lam E W, La T N. DP and E2F proteins: coordinating transcription with cell cycle progression. Curr Opin Cell Biol 1994; 6(6):859-66.
Lee J O, Russo A A, Pavletich N P. Structure of the retinoblastoma tumour-suppressor pocket domain bound to a peptide from HPV E7. Nature 1998; 391(6670):859-65.
Macleod K. Tumor suppressor genes. Curr Opin Genet Dev 2000; 10(1):81-93.
Miel, H, Rault, S. Total deprotection of N,N′-bis(tert-butoxycarbonyl)guanidines using SnCl4. Tetrahedron Lett. 1997, 38, 7865-7866.
Morris E J, Dyson N J. Retinoblastoma protein partners. Adv Cancer Res 2001; 82:1-54.
Muller H, Bracken A P, Vernell R, et al. E2Fs regulate the expression of genes involved in differentiation, development, proliferation, and apoptosis. Genes Dev 2001; 15(3):267-85.
Muller H, Helin K. The E2F transcription factors: key regulators of cell proliferation. Biochim Biophys Acta 2000; 1470(0:M1-M12.
Nevins J R. Cell cycle targets of the DNA tumor viruses. Curr. Opin. Genet. Devel. 1994, 4(1), 130-134.
Nevins J R. Disruption of cell-cycle control by viral oncoproteins. Biochemical Society

Transactions 1993; 21(4):935-8.
Nevins J R. E2F: a link between the Rb tumor suppressor protein and viral oncoproteins. Science 1992, 258(5081), 424-429.
Nevins, J R. The Rb/E2F pathway and cancer, Hum. Mol. Genet. 2001, 10, 699-703.
Norbury C, Nurse P. Animal cell cycles and their control. Ann Rev Biochem 1992; 61:441-70.
Paggi, M G, Baldi, A, Bonetto, F, Giordano, A. Retinoblastoma protein family in cell cycle and cancer: A review. J. Cell. Biochem. 1996, 62, 418-430.
Prives C, Hall P A. The p53 pathway. J Pathol 1999; 187(1):112-26.
Reddy G P. Cell cycle: regulatory events in G1->S transition of mammalian cells. J. Cell. Biochem. 1994, 54(4), 379-386.
Reed S I. Control of the G1/S transition. Cancer Surveys 1997, 29, 7-23.
Ren B, Cam H, Takahashi Y, et al. E2F integrates cell cycle progression with DNA repair, replication, and G(2)/M checkpoints. Genes Dev 2002; 16(2):245-56.
Rini, B I. Sorafenib. Expert Opinion on Pharmacotherapy 2006, 7, 453-461.
Rudin, C M, HolmLund, J, Fleming, G F, Mani, S, Stadler, W M. et al. Phase I trial of ISIS 5132, an antisense oligonucleotide inhibitor of c-raf-1, administered by 24-hour weekly infusion to patients with advanced cancer. Clin. Cancer Res. 2001, 7, 1214-1220.
Sager R. Tumor suppressor genes in the cell cycle. Curr Opin Cell Biol 1992; 4155-160.
Sager R. Tumor suppressor genes: The puzzle and the promise. Science 1989; 246:1406-12.
Sherr C J, McCormick F. The RB and p53 pathways in cancer. Cancer Cell 2002; 2(2):103-12.
Sherr C J, Roberts J M. Inhibitors of mammalian G1 cyclin-dependent kinases. Genes Dev. 1995; 9(10):1149-63.
Sherr C J. Cell cycle control and cancer. Harvey Lect 2000; 96:73-92.
Sherr C J. Mammalian G1 cyclins and cell cycle progression. Proc Assoc Am Physicians 1995; 107(2):181-6.
Sherr C J. The ins and outs of RB: Coupling gene expression to the cell cycle clock. Trends Cell Biol. 1994; 4, 15-18.
Sherr C J. The ins and outs of RB: Coupling gene expression to the cell cycle clock. Trends Cell Biol. 1994; 4:15-8.
Slansky J E, Farnham P J. Introduction to the E2F family: protein structure and gene regulation. Curr Top Microbiol Immunol 1996; 208(1):1-30.
Stiegler P, Kasten M, Giordano A. The RB family of cell cycle regulatory factors. J Cell Biochem Suppl 1998; 31:30-6.
Takahashi Y, Rayman J B, Dynlacht B D. Analysis of promoter binding by the E2F and pRb families in vivo: distinct E2F proteins mediate activation and repression. Genes Dev 2000; 14:804-16.
Taya Y, Yasuda H, Kamijo M, et al. In vitro phosphorylation of the tumor suppressor gene RB protein by mitosis-specific histone H1 kinase. Biochem Biophys Res Commit 1989; 164(1):580-6.
Taya Y. RB kinases and RB-binding proteins: new points of view. Trends Biochem Sci 1997; 22(1):14-7.
Taylor W R, Stark G R. Regulation of the G2/M transition by p53. Oncogene 2001; 20(15):1803-15.
Tonini T, Hillson C, Claudio P P. Interview with the retinoblastoma family members: do they help each other? J Cell Physiol 2002; 192(2):138-50.

Trimarchi J M, Lees J A. Transcription: Sibling rivalry in the E2F family. Nat Rev Mol Cell Biol 2002; 3(1):11-20.
Wang S, Ghosh R, Chellappan S. Raf-1 physically interacts with Rb and regulates its function: A link between mitogenic signaling and cell cycle regulation. Mol Cell Biol 1998; 18(12):7487-98.
Wang S, Nath N, Minden A, Chellappan S. Regulation of Rb and E2F by signal transduction cascades: divergent effects of JNK1 and p38 kinases. Embo J 1999; 18(6):1559-70.
Wang, S, Ghosh, R N, Chellappan, S P. Raf-1 physically interacts with Rb and regulates its function: a link between mitogenic signaling and cell cycle regulation. Mol. Cell. Biol. 1998, 18, 7487-7498.
Weinberg R A. E2F and cell proliferation: a world turned upside down. Cell 1996; 85(4):457-9.
Weinberg, R A. The Retinoblastoma Protein and Cell-Cycle Control. Cell 1995, 81, 323-330.
Welch P J, Wang J Y. Disruption of retinoblastoma protein function by coexpression of its C pocket fragment. Genes Dev. 1995; 9(1):31-46.
Welch P J, Wang J Y J. A C-terminal protein-binding domain in the retinoblastoma protein regulates nuclear c-Abl tyrosine in the cell cycle. Cell 1993; 75:779-90.

Wilhelm S, Carter C, Lynch M, et al. Discovery and development of sorafenib: a multikinase inhibitor for treating cancer. Nat. Rev. Drug Discov. 2006, 5(10), 8358-8344.
Wilhelm, S M, Carter, C, Tang, L Y, Wilkie, D, McNabola, A. et al. BAY 43-9006 exhibits broad spectrum oral antitumor activity and targets the RAF/MEK/ERK pathway and receptor tyrosine kinases involved in tumor progression and angiogenesis. Cancer Res. 2004, 64, 7099-7109.
Yin H, Hamilton A D. Strategies for targeting protein-protein interactions with synthetic agents. Angew. Chem. Int. Ed. Engl. 2005, 44(27), 4130-4163.
Yong, Y F, Kowalski, J A, Lipton, M A. Facile and efficient guanylation of amines using thioureas and Mukaiyama's reagent. J. Org. Chem. 1997, 62, 1540-1542.

1. A compound according to formula (I): 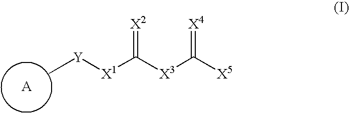
or a salt thereof, wherein:
Group A is substituted phenyl, optionally substituted 6-membered heteroaryl, or optionally substituted fused bicyclic 9-10 membered aryl or heteroaryl;
Y is optionally substituted methylene;
X¹ is —O—, —S—, or optionally substituted —NH—;
X³ is —O—, —S—, optionally substituted —NH— or optionally substituted methylene;
X² is S or optionally substituted NH;
X⁴ is S or optionally substituted NH;
or X² and X⁴ are both N and are linked together through an optionally substituted alkyl, alkenyl, heteroalkyl, or heteroalkenyl linking group, thereby forming an optionally substituted 5-7 membered heteroaryl or heterocyclyl ring;
X⁵ is an optionally substituted —NH₂ or 3-7 membered heteroaryl or heterocyclyl ring;
wherein
each optionally substitutable carbon is optionally substituted with —F, —Cl, —Br, —I, —CN, —NO₂, —R^{a}, —OR^{a}, —C(O)R^{a}, —OC(O)R^{a}, —C(O)OR^{a}, —SR^{a}, —C(S)R^{a}, —OC(S)R^{a}, —C(S)OR^{a}, —C(O)SR^{a}, —C(S)SR^{a}, —S(O)R^{a}, —SO₂R^{a}, —SO₃R^{a}, —OSO₂R^{a}, —OSO₃R^{a}, —PO₂R^{a}R^{b}, —OPO₂R^{a}R^{b}, —PO₃R^{a}R^{b}, —OPO₃R^{a}R^{b}, —N(R^{a}R^{b}), —C(O)N(R^{a}R^{b}), —C(O)NR^{a}NR^{b}SO₂R^{c}, —C(O)NR^{a}SO₂R^{c}, —C(O)NR^{a}CN, —SO₂N(R^{a}R^{b}), —NR^{a}SO₂R^{b}, —NR^{c}C(O)R^{a}, —NR^{c}C(O)OR^{a}, —NR^{c}C(O)N(R^{a}R^{b}), —C(NR^{c})—N(R^{a}R^{b}), —NR^{d}—C(NR^{c})—N(R^{a}R^{b}), —NR^{a}N(R^{a}R^{b}), —CR^{c}═CR^{a}R^{b}, —C≡CR^{a}, ═O, ═S, ═CR^{a}R^{b}, ═NR^{a}, ═NOR^{a}, or ═NNR^{a}, or two optionally substitutable carbons are linked with C₁₋₃ alkylenedioxy;
each optionally substitutable nitrogen is:
optionally substituted with —CN, —NO₂, —R^{a}, —OR^{a}, —C(O)R^{a}, —C(O)R^{a}-aryl, —OC(O)R^{a}, —C(O)OR^{a}, —SR^{a}, —S(O)R^{a}, —SO₂R^{a}, —SO₃R^{a}, —N(R^{a}R^{b}), —C(O)N(R^{a}R^{b}), —C(O)NR^{a}NR^{b}SO₂R^{c}, —C(O)NR^{a}SO₂R^{c}, —C(O)NR^{a}CN, —SO₂N(R^{a}R^{b}), —NR^{a}SO₂R^{b}, —NR^{c}C(O)R^{a}, —NR^{c}C(O)OR^{a}, —NR^{c}C(O)N(R^{a}R^{b}), or oxygen to form an N-oxide; and
is optionally protonated or quaternary substituted with a nitrogen substituent, thereby carrying a positive charge which is balanced by a counterion; and
wherein each of R^{a}, R^{b}, R^{c} and R^{d} is independently —H, alkyl, haloalkyl, aralkyl, aryl, heteroaryl, heterocyclyl, or cycloaliphatic, or
in any occurrence of —N(R^{a}R^{b}), R^{a} and R^{b} taken together with the nitrogen to which they are attached optionally form an optionally substituted heterocyclic group
with the proviso that when X¹ is NH, X² is NH, X³ is NH, X⁴ is NH, X⁵ is NH₂, and Y is CH₂, then ring A is other than 2-trifluoromethylphenyl, 3-methoxyphenyl, 3-nitrophenyl, 3-trifluoromethylphenyl, 3-vinylphenyl, 4-t-butylphenyl, 4-chlorophenyl, 4-fluorophenyl, 4-methoxyphenyl, 4-methylphenyl, 4-nitrophenyl, 4-trifluoromethylphenyl, 4-vinylphenyl, 3,4-dichlorophenyl, 3,5-ditrifluoromethylphenyl, and 2-hydroxy-5-nitrophenyl.

2. A compound according to claim 1, or a salt thereof, wherein Group A is substituted phenyl or optionally substituted naphthyl or pyridyl.

3. A compound according to claim 1, or a salt thereof, wherein in Group A, an unsubstituted ring atom is adjacent to the ring atom attached to Y.

4. A compound according to claim 1, or a salt thereof, wherein Y is C(O), C(S), or methylene optionally substituted with hydroxyl, C₁₋₆ alkyl, C₁₋₆ alkoxy, C₁₋₆ haloalkyl, C₁₋₆ haloalkoxy, C₁₋₆ alkyl substituted with aryl, aryl, heteroaryl, heterocyclyl, or cycloaliphatic.

5. A compound according to claim 1, or a salt thereof, wherein Y is C(O), or methylene optionally substituted with hydroxyl, C₁₋₆ alkyl, C₁₋₆ alkoxy, C₁₋₆ haloalkyl, C₁₋₆ haloalkoxy, or C₁₋₆ alkyl substituted with aryl.

6. A compound according to claim 1, or a salt thereof, wherein Y is methylene optionally substituted with hydroxyl, C₁₋₆ alkyl, C₁₋₆ alkoxy, or C₁₋₆ alkyl substituted with aryl.

7. A compound according to claim 1, or a salt thereof, wherein Y is methylene optionally substituted with C₁₋₃ alkyl.

8. A compound according to claim 1, or a salt thereof, wherein Y is methylene.

9. A compound according to claim 1, or a salt thereof, wherein the compound is represented by the following structural formula (Ia): 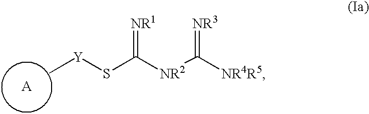
or a salt thereof, wherein:
R¹ is hydrogen, hydroxyl, C₁₋₆ alkyl, C₁₋₆ alkoxy, C₁₋₆ haloalkyl, C₁₋₆ haloalkoxy, C₁₋₆ alkyl substituted with aryl, aryl, heteroaryl, heterocyclyl, or cycloaliphatic;
R² is hydrogen, hydroxyl, C₁₋₆ alkyl, C₁₋₆ alkoxy, C₁₋₆ haloalkyl, C₁₋₆ haloalkoxy, C₁₋₆ alkyl substituted with aryl, aryl, heteroaryl, heterocyclyl, or cycloaliphatic;
R³ is hydrogen, hydroxyl, C₁₋₆ alkyl, C₁₋₆ alkoxy, C₁₋₆ haloalkyl, C₁₋₆ haloalkoxy, C₁₋₆ alkyl substituted with aryl, aryl, heteroaryl, heterocyclyl, or cycloaliphatic;
R⁴ is hydrogen, hydroxyl, C₁₋₆ alkyl, C₁₋₆ alkoxy, C₁₋₆ haloalkyl, C₁₋₆ haloalkoxy, C₁₋₆ alkyl substituted with aryl, aryl, heteroaryl, heterocyclyl, or cycloaliphatic; and
R⁵ is hydrogen, hydroxyl, C₁₋₆ alkyl, C₁₋₆ alkoxy, C₁₋₆ haloalkyl, C₁₋₆ haloalkoxy, C₁₋₆ alkyl substituted with aryl, aryl, heteroaryl, heterocyclyl, or cycloaliphatic.

10. A compound according to claim 9, or a salt thereof, wherein:
R¹ is hydrogen, hydroxyl, C₁₋₆ alkyl, C₁₋₆ alkoxy, C₁₋₆ haloalkyl, C₁₋₆ haloalkoxy, or C₁₋₆ alkyl substituted with aryl;
R² is hydrogen, hydroxyl, C₁₋₆ alkyl, C₁₋₆ alkoxy, C₁₋₆ haloalkyl, C₁₋₆ haloalkoxy, or C₁₋₆ alkyl substituted with aryl;
R³ is hydrogen, hydroxyl, C₁₋₆ alkyl, C₁₋₆ alkoxy, C₁₋₆ haloalkyl, C₁₋₆ haloalkoxy, or C₁₋₆ alkyl substituted with aryl;
R⁴ is hydrogen, hydroxyl, C₁₋₆ alkyl, C₁₋₆ alkoxy, C₁₋₆ haloalkyl, C₁₋₆ haloalkoxy, or C₁₋₆ alkyl substituted with aryl; and
R⁵ is hydrogen, hydroxyl, C₁₋₆ alkyl, C₁₋₆ alkoxy, C₁₋₆ haloalkyl, C₁₋₆ haloalkoxy, or C₁₋₆ alkyl substituted with aryl.

11. A compound according to claim 9, or a salt thereof, wherein:
R¹ is hydrogen, hydroxyl, C₁₋₆ alkyl, C₁₋₆ alkoxy, or C₁₋₆ alkyl substituted with aryl;
R² is hydrogen, hydroxyl, C₁₋₆ alkyl, C₁₋₆ alkoxy, or C₁₋₆ alkyl substituted with aryl;
R³ is hydrogen, hydroxyl, C₁₋₆ alkyl, C₁₋₆ alkoxy, or C₁₋₆ alkyl substituted with aryl;
R⁴ is hydrogen, hydroxyl, C₁₋₆ alkyl, C₁₋₆ alkoxy, or C₁₋₆ alkyl substituted with aryl; and
R⁵ is hydrogen, hydroxyl, C₁₋₆ alkyl, C₁₋₆ alkoxy, or C₁₋₆ alkyl substituted with aryl.

12. A compound according to claim 9, or a salt thereof, wherein:
R¹ is hydrogen or C₁₋₃ alkyl;
R² is hydrogen or C₁₋₃ alkyl;
R³ is hydrogen or C₁₋₃ alkyl;
R⁴ is hydrogen or C₁₋₃ alkyl; and
R⁵ is hydrogen or C₁₋₃ alkyl.

13. A compound according to claim 9, or a salt thereof, wherein:
R¹ is hydrogen;
R² is hydrogen;
R³ is hydrogen;
R⁴ is hydrogen; and
R⁵ is hydrogen.

14. A compound according to claim 9, or a salt thereof, wherein A is substituted phenyl.

15. A compound according to claim 14, or a salt thereof, wherein:
Y is methylene;
R¹ is hydrogen;
R² is hydrogen;
R³ is hydrogen;
R⁴ is hydrogen; and
R⁵ is hydrogen.

16. A compound according to claim 9, or a salt thereof, wherein A is optionally substituted naphthyl.

17. A compound according to claims 16, or a salt thereof, wherein:
Y is methylene;
R¹ is hydrogen;
R² is hydrogen;
R³ is hydrogen;
R⁴ is hydrogen; and
R⁵ is hydrogen.

18. A compound according to claim 16, or a salt thereof, wherein A is optionally substituted 1-naphthyl.

19. A compound according to claim 16, or a salt thereof, wherein A is optionally substituted 2-naphthyl.

20. (canceled)

21. A compound according to claim 1, or a salt thereof, wherein one, two or three substitutable carbons in Group A are substituted with a substituent independently selected from —F, —Cl, —Br, —I, —CN, —NO₂, C₁₋₆ alkyl, C₁₋₆ alkoxy, —CF₃, and C₁₋₆ haloalkoxy, or two substitutable carbons are linked with C₁₋₂ alkylenedioxy.

22. A compound according to claim 21, or a salt thereof, wherein Group A is phenyl, wherein one, two or three substitutable carbons of the phenyl are substituted with a substituent independently selected from —F, —Cl, —Br, —I, —CN, —NO₂, C₁₋₆ alkyl, C₁₋₆ alkoxy, —CF₃, and C₁₋₆ haloalkoxy, or two substitutable carbons are linked with C₁₋₂ alkylenedioxy.

23. A compound according to claim 22, or a salt thereof, wherein the compound is selected from the following compounds: 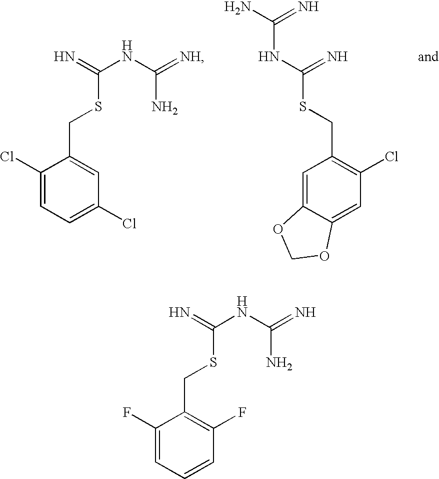
and salts thereof.

24. A compound according to claim 1, or a salt thereof, wherein Group A is phenyl unsubstituted at its 6-position.

25. A compound according to claim 1, or a salt thereof, wherein Group A is 2,4-substituted phenyl.

26. A compound according to claim 1, or a salt thereof, wherein Group A is phenyl monosubstituted at its 2, 3, or 4 positions or independently disubstituted at its 2,3, 2,4, 2,5 or 3,4 positions with —F, —Cl, —Br, —NO₂, C₁₋₆ alkyl, or —CF₃.

27. A compound according to claim 1, or a salt thereof, wherein Group A is phenyl independently disubstituted at its 2,3, 2,4, 3,4, or 2,5 positions with —NO₂, —Cl, —F or —CF₃.

28. A compound according to claim 1, or a salt thereof, wherein Group A is phenyl monosubstituted at its 2, 3, or 4 position with —NO₂, —Cl or —F.

29. A compound according to claim 1, or a salt thereof, wherein Group A is phenyl independently disubstituted at its 2,4 positions with —NO₂, —Cl or —F.

30. A compound according to claim 29, or a salt thereof, wherein the compound is selected from the following compounds: 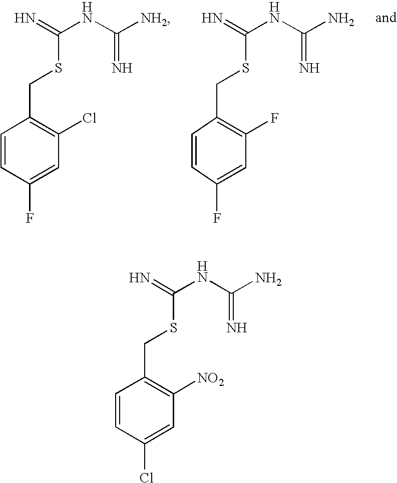
and salts thereof.

31. A compound according to claim 29, or a salt thereof, wherein the compound is the following compound, 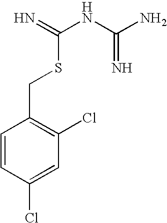
or a salt thereof.

32. A compound according to claim 1, or a salt thereof, wherein Group A is unsubstituted 2-naphthyl or 1-substituted 2-naphthyl.

33. A compound according to claim 1, or a salt thereof, wherein Group A is naphthyl optionally substituted with one or more of —F, —Cl, —Br, —NO₂, C₁₋₆ alkyl, or —CF₃.

34. A compound according to claim 1, or a salt thereof, wherein Group A is naphthyl optionally monosubstituted with —F, —Cl, —Br, —NO₂, or —CF₃.

35. A compound according to claim 1, or a salt thereof, wherein Group A is naphthyl optionally monosubstituted with —F, —Cl, or —Br.

36. A compound according to claim 34, or a salt thereof, wherein the compound is selected from the following compounds: 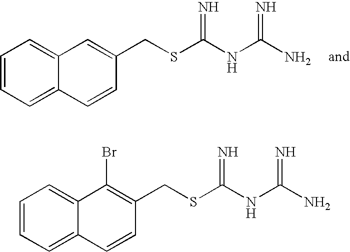
and salts thereof.

37. A compound according to formula (II): 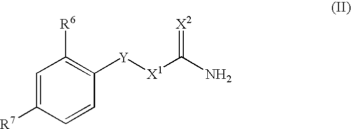
or a salt thereof, wherein:
Y is optionally substituted methylene;
X¹ is —O—, —S—, or optionally substituted —NH—;
X² is S or optionally substituted NH; and
R⁶ and R⁷ are independently —F, —Cl, —Br, —I, —NO₂, —CN, —CF₃, or C₁-C₆ alkoxy, provided that R⁶ and R⁷ are not both —Cl and R⁶ and R⁷ are not both —CF₃;
with the further proviso that when Y is —CH₂—, X¹ is S and X² is NH, then R⁶ and R⁷ are not both —F, R⁶ and R⁷ are not both —Br, R⁶ and R⁷ are not both —I, R⁶ and R⁷ are not both —NO₂; and R⁶ and R⁷ are not both —CH₃;
wherein
each optionally substitutable carbon is optionally substituted with —F, —Cl, —Br, —I, —CN, —NO₂, —R^{a}, —OR^{a}, —C(O)R^{a}, —OC(O)R^{a}, —C(O)OR^{a}, —SR^{a}, —C(S)R^{a}, —OC(S)R^{a}, —C(S)OR^{a}, —C(O)SR^{a}, —C(S)SR^{a}, —S(O)R^{a}, —SO₂R^{a}, —SO₃R^{a}, —OSO₂R^{a}, OSO₃R^{a}, —PO₂R^{a}R^{b}, —OPO₂R^{a}R^{b}, —PO₃R^{a}R^{b}, —OPO₃R^{a}R^{b}, —N(R^{a}R^{b}), —C(O)N(R^{a}R^{b}), —C(O)NR^{a}NR^{b}SO₂R^{c}, —C(O)NR^{a}SO₂R^{c}, —C(O)NR^{a}CN, —SO₂N(R^{a}R^{b}), —NR^{a}SO₂R^{b}, —NR^{c}C(O)R^{a}, —NR^{c}C(O)OR^{a}, —NR^{c}C(O)N(R^{a}R^{b}), —C(NR^{c})—N(R^{a}R^{b}), —NR^{d}—C(NR^{c})—N(R^{a}R^{b}), —NR^{a}N(R^{a}R^{b}), —CR^{c}═CR^{a}R^{b}, —C≡CR^{a}, ═O, ═S, ═CR^{a}R^{b}, ═NR^{a}, ═NOR^{a}, or ═NNR^{a}, or two optionally substitutable carbons are linked with C₁₋₃ alkylenedioxy;
each optionally substitutable nitrogen is:
optionally substituted with —CN, —NO₂, —R^{a}, —OR^{a}, —C(O)R^{a}, —C(O)R^{a}-aryl, —OC(O)R^{a}, —C(O)OR^{a}, —SR^{a}, —S(O)R^{a}, —SO₂R^{a}, —SO₃R^{a}, —N(R^{a}R^{b}), —C(O)N(R^{a}R^{b}), —C(O)NR^{a}NR^{b}SO₂R^{c}, —C(O)NR^{a}SO₂R^{c}, —C(O)NR^{a}CN, —SO₂N(R^{a}R^{b}), —NR^{a}SO₂R^{b}, —NR^{c}C(O)R^{a}, —NR^{c}C(O)OR^{a}, —NR^{c}C(O)N(R^{a}R^{b}), or oxygen to form an N-oxide; and
optionally is protonated or quaternary substituted with a nitrogen substituent, thereby carrying a positive charge which is balanced by a pharmaceutically acceptable counterion; and
wherein each of R^{a}, R^{b}, R^{c} and R^{d} is independently —H, alkyl, haloalkyl, aralkyl, aryl, heteroaryl, heterocyclyl, or cycloaliphatic, or
in any occurrence of —N(R^{a}R^{b}), R^{a} and R^{b} taken together with the nitrogen to which they are attached optionally form an optionally substituted heterocyclic group.

38-42. (canceled)

43. A compound according to claim 37, or a salt thereof, wherein the compound is represented by the following structural formula: 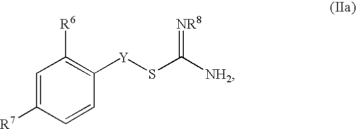
or a salt thereof, wherein R⁸ is hydrogen, hydroxyl, C₁₋₆ alkyl, C₁₋₆ alkoxy, C₁₋₆ haloalkyl, C₁₋₆ haloalkoxy, C₁₋₆ alkyl substituted with aryl, aryl, heteroaryl, heterocyclyl, or cycloaliphatic.

44-47. (canceled)

48. A compound according to claim 43, or a salt thereof, wherein the compound is selected from the group consisting of: 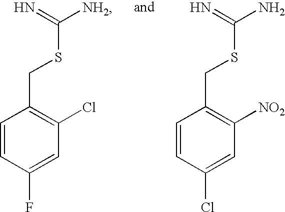
and salts thereof.

49. A pharmaceutical composition comprising a compound of claim 1, or a pharmaceutically acceptable salt thereof and a pharmaceutically acceptable carrier.

50. A method of treating or ameliorating a cell proliferation disorder, comprising administering to a subject in need of such treatment an effective amount of a compound according to formula (I): 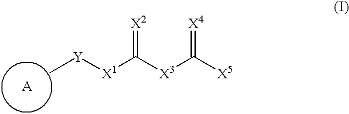
or a pharmaceutically acceptable salt thereof, wherein:
Group A is substituted phenyl, optionally substituted 6-membered heteroaryl, or optionally substituted fused bicyclic 9-10 membered aryl or heteroaryl;
Y is optionally substituted methylene;
X¹ is —O—, —S—, or optionally substituted —NH—;
X³ is —O—, —S—, optionally substituted —NH— or optionally substituted methylene;
X² is S or optionally substituted NH;
X⁴ is S or optionally substituted NH;
or X² and X⁴ are both N and are linked together through an optionally substituted alkyl, alkenyl, heteroalkyl, or heteroalkenyl linking group, thereby forming an optionally substituted 5-7 membered heteroaryl or heterocyclyl ring;
X⁵ is an optionally substituted —NH₂ or 3-7 membered heteroaryl or heterocyclyl ring;
wherein
each optionally substitutable carbon is optionally substituted with —F, —Cl, —Br, —I, —CN, —NO₂, —R^{a}, —OR^{a}, —C(O)R^{a}, —OC(O)R^{a}, —C(O)OR^{a}, —SR^{a}, —C(S)R^{a}, —OC(S)R^{a}, —C(S)OR^{a}, —C(O)SR^{a}, —C(S)SR^{a}, —S(O)R^{a}, —SO₂R^{a}, —SO₃R^{a}, —OSO₂R^{a}, —OSO₃R^{a}, —PO₂R^{a}R^{b}, —OPO₂R^{a}R^{b}, —PO₃R^{a}R^{b}, —OPO₃R^{a}R^{b}, —N(R^{a}R^{b}), —C(O)N(R^{a}R^{b}), —C(O)NR^{a}NR^{b}SO₂R^{c}, —C(O)NR^{a}SO₂R^{c}, —C(O)NR^{a}CN, —SO₂N(R^{a}R^{b}), —NR^{a}SO₂R^{b}, —NR^{c}C(O)R^{a}, —NR^{c}C(O)OR^{a}, —NR^{c}C(O)N(R^{a}R^{b}), —C(NR^{c})—N(R^{a}R^{b}), —NR^{d}—C(NR^{c})—N(R^{a}R^{b}), —NR^{a}N(R^{a}R^{b}), —CR^{c}═CR^{a}R^{b}, —C≡CR^{a}, ═O, ═S, ═CR^{a}R^{b}, ═NR^{a}, ═NOR^{a}, or —NNR^{a}, or two optionally substitutable carbons are linked with C₁₋₃ alkylenedioxy;
each optionally substitutable nitrogen is:
optionally substituted with —CN, —NO₂, —R^{a}, —OR^{a}, —C(O)R^{a}, —C(O)R^{a}-aryl, —OC(O)R^{a}, —C(O)OR^{a}, —SR^{a}, —S(O)R^{a}, —SO₂R^{a}, —SO₃R^{a}, —N(R^{a}R^{b}), —C(O)N(R^{a}R^{b}), —C(O)NR^{a}NR^{b}SO₂R^{c}, —C(O)NR^{a}SO₂R^{c}, —C(O)NR^{a}CN, —SO₂N(R^{a}R^{b}), —NR^{a}SO₂R^{b}, —NR^{c}C(O)R^{a}, —NR^{c}C(O)OR^{a}, —NR^{c}C(O)N(R^{a}R^{b}), or oxygen to form an N-oxide; and
is optionally protonated or quaternary substituted with a nitrogen substituent, thereby carrying a positive charge which is balanced by a counterion; and
wherein each of R^{a}, R^{b}, R^{c} and R^{d} is independently —H, alkyl, halo alkyl, aralkyl, aryl, heteroaryl, heterocyclyl, or cycloaliphatic, or
in any occurrence of —N(R^{a}R^{b}), R^{a} and R^{b} taken together with the nitrogen to which they are attached optionally form an optionally substituted heterocyclic group.

51-53. (canceled)

54. A method of treating or ameliorating a cell proliferation disorder, comprising administering to a subject in need of such treatment an effective amount of a compound according to formula (II): 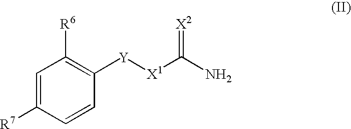
or a pharmaceutically acceptable salt thereof, wherein:
Y is optionally substituted methylene;
X¹ is —O—, —S—, or optionally substituted —NH—;
X² is S or optionally substituted NH; and
R⁶ and R⁷ are independently —F, —Cl, —Br, —I, —NO₂, —CN, —CF₃, or C₁-C₆ alkoxy, provided that R⁶ and R⁷ are not both —Cl and R⁶ and R⁷ are not both —CF₃;
wherein
each optionally substitutable carbon is optionally substituted with —F, —Cl, —Br, —I, —CN, —NO₂, —R^{a}, —OR^{a}, —C(O)R^{a}, —OC(O)R^{a}, —C(O)OR^{a}, —SR^{a}, —C(S)R^{a}, —OC(S)R^{a}, —C(S)OR^{a}, —C(O)SR^{a}, —C(S)SR^{a}, —S(O)R^{a}, —SO₂R^{a}, —SO₃R^{a}, —OSO₂R^{a}, —OSO₃R^{a}, —PO₂R^{a}R^{b}, —OPO₂R^{a}R^{b}, —PO₃R^{a}R^{b}, —OPO₃R^{a}R^{b}, —N(R^{a}R^{b}), —C(O)N(R^{a}R^{b}), —C(O)NR^{a}NR^{b}SO₂R^{c}, —C(O)NR^{a}SO₂R^{c}, —C(O)NR^{a}CN, —SO₂N(R^{a}R^{b}), —NR^{a}SO₂R^{b}, —NR^{c}C(O)R^{a}, —NR^{c}C(O)OR^{a}, —NR^{c}C(O)N(R^{a}R^{b}), —C(NR^{c})—N(R^{a}R^{b}), —NR^{d}—C(NR^{c})—N(R^{a}R^{b}), —NR^{a}N(R^{a}R^{b}), —CR^{c}═CR^{a}R^{b}, —C≡CR^{a}, ═O, ═S, ═CR^{a}R^{b}, ═NR^{a}, ═NOR^{a}, or ═NNR^{a}, or two optionally substitutable carbons are linked with C₁₋₃ alkylenedioxy;
each optionally substitutable nitrogen is:
optionally substituted with —CN, —NO₂, —R^{a}, —OR^{a}, —C(O)R^{a}, —C(O)R^{a}-aryl, —OC(O)R^{a}, —C(O)OR^{a}, —SR^{a}, —S(O)R^{a}, —SO₂R^{a}, —SO₃R^{a}, —N(R^{a}R^{b}), —C(O)N(R^{a}R^{b}), —C(O)NR^{a}NR^{b}SO₂R^{c}, —C(O)NR^{a}SO₂R^{c}, —C(O)NR^{a}CN, —SO₂N(R^{a}R^{b}), —NR^{a}SO₂R^{b}, —NR^{c}C(O)R^{a}, —NR^{c}C(O)OR^{a}, —NR^{c}C(O)N(R^{a}R^{b}), or oxygen to form an N-oxide; and
optionally is protonated or quaternary substituted with a nitrogen substituent, thereby carrying a positive charge which is balanced by a pharmaceutically acceptable counterion; and
wherein each of R^{a}, R^{b}, R^{c} and R^{d} is independently —H, alkyl, haloalkyl, aralkyl, aryl, heteroaryl, heterocyclyl, or cycloaliphatic, or
in any occurrence of —N(R^{a}R^{b}), R^{a} and R^{b} taken together with the nitrogen to which they are attached optionally form an optionally substituted heterocyclic group.

55-63. (canceled)

64. A method of inhibiting proliferation of a cell, comprising contacting the cell with an effective amount of a compound according to claim 1, or a salt thereof.

65-82. (canceled)
